SEQUENCE LISTING (117 sequences)

SEQ ID NO: 1 - DNA, 1389 bp, Mus sp.
  CDS (1)..(1389)
atgaacaggcttacttcctcattgctgctgctgattgtccctgcatatgtcctgtcccaggttactctgaaagagtctggccctgggatattgcagccctcccagaccctcagtctgacttgttctttctctgggttttcactgagcacttctggtatgggtgtgagctggattcgtcagccttcaggaaagggtctggagtggctggcacacatttactgggatgatgacaagcgctataacccatccctgaagagccggctcacaatctccaaggatacctccagcaaccaggtattcctcaagatcaccagtgtggacactgcagatactgccacatactactgtgctcgagattactacgctagtagctttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaagtga

SEQ ID NO: 2 - protein, 462 aa, Mus sp.
MNRLTSSLLLLIVPAYVLSQVTLKESGPGILQPSQTLSLTCSFSGFSLSTSGMGVSWIRQPSGKGLEWLAHIYWDDDKRYNPSLKSRLTISKDTSSNQVFLKITSVDTADTATYYCARDYYASSFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 3 - DNA, 705 bp, Mus sp.
  CDS (1)..(705)
atggttttcacacctcagattcttggacttatgcttttctggatttcagcctccagaggtgatattgtgctaactcagtctccagccaccctgtctgtgactccaggagatagagtcagtctttcctgcagggccagtcaaagtattagcaactacctacactggtatcaacaaaaatcacatgagtctccaaggcttctcatcaagtatgcttcccagtccatctctgggatcccctccaggttcagtggcagtggatcagggacagatttcattctcagtttcaacagtgtggagactgaagattttggaatgtatttctgtcaacagagtaacagctggcctttcacgttcggctcggggacaaaattggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaa

SEQ ID NO: 4 - protein, 234 aa, Mus sp.
MVFTPQILGLMLFWISASRGDIVLTQSPATLSVTPGDRVSLSCRASQSISNYLHWYQQKSHESPRLLIKYASQSISGIPSRFSGSGSGTDFILSFNSVETEDFGMYFCQQSNSWPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - DNA, 442 bp, Mus sp.
  CDS (29)..(442)
tgaaggagtagaaaactagtgtgcagatatgaacaggcttacttcctcattgctgctgctgattgtccctgcatatgtcctgtcccaggttactctgaaagagtctggccctgggatattgcagccctcccagaccctcagtctgacttgttctttctctgggttttcactgagcacttctggtatgggtgtgagctggattcgtcagccttcaggaaagggtctggagtggctggcacacatttactgggatgatgacaagcgctataacccatccctgaagagccggctcacaatctccaaggatacctccagcaaccaggtattcctcaagatcaccagtgtggacactgcagatactgccacatactactgtgctcgagattactacgctagtagctttgactactggggccaaggcaccactctcacagtctcctca

SEQ ID NO: 6 - protein, 138 aa, Mus sp.
MNRLTSSLLLLIVPAYVLSQVTLKESGPGILQPSQTLSLTCSFSGFSLSTSGMGVSWIRQPSGKGLEWLAHIYWDDDKRYNPSLKSRLTISKDTSSNQVFLKITSVDTADTATYYCARDYYASSFDYWGQGTTLTVSS

SEQ ID NO: 7 - DNA, 401 bp, Mus sp.
  CDS (18)..(401)
tcagggaaagctcgaagatggttttcacacctcagattcttggacttatgcttttctggatttcagcctccagaggtgatattgtgctaactcagtctccagccaccctgtctgtgactccaggagatagagtcagtctttcctgcagggccagtcaaagtattagcaactacctacactggtatcaacaaaaatcacatgagtctccaaggcttctcatcaagtatgcttcccagtccatctctgggatcccctccaggttcagtggcagtggatcagggacagatttcattctcagtttcaacagtgtggagactgaagattttggaatgtatttctgtcaacagagtaacagctggcctttcacgttcggctcggggacaaaattggaaataaaacgg

SEQ ID NO: 8 - protein, 128 aa, Mus sp.
MVFTPQILGLMLFWISASRGDIVLTQSPATLSVTPGDRVSLSCRASQSISNYLHWYQQKSHESPRLLIKYASQSISGIPSRFSGSGSGTDFILSFNSVETEDFGMYFCQQSNSWPFTFGSGTKLEIKR

SEQ ID NO: 9 - protein, 7 aa, Mus sp.
TSGMGVS

SEQ ID NO: 10 - protein, 16 aa, Mus sp.
HIYWDDDKRYNPSLKS

SEQ ID NO: 11 - protein, 9 aa, Mus sp.
DYYASSFDY

SEQ ID NO: 12 - protein, 11 aa, Mus sp.
RASQSISNYLH

SEQ ID NO: 13 - protein, 7 aa, Mus sp.
YASQSIS

SEQ ID NO: 14 - protein, 9 aa, Mus sp.
QQSNSWPFT

SEQ ID NO: 15 - DNA, 1395 bp, Mus sp.
  CDS (1)..(1395)
atggactccaggctcaatttagttttccttgtccttattttaaaaggtgtccagtgtgatgtgcagctggtggagtctgggggaggcttagtgcagcctggagggtcccggaaactctcctgtgcagcctctggattcactttcagtaactttggaatgcactgggttcgtcaggctccagagaagggactggagtgggtcgcatacattagtagtggcagtagtaccatctactatgcagacacagtgaagggccgattcaccatctccagagacaatcccaagaacaccctgttcctgcaaatgaccagtctaaggtctgaggacacggccatgtattactgtgcaagatatgattacgacgatgattactatgctatggaatactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaagtga

SEQ ID NO: 16 - protein, 464 aa, Mus sp.
MDSRLNLVFLVLILKGVQCDVQLVESGGGLVQPGGSRKLSCAASGFTFSNFGMHWVRQAPEKGLEWVAYISSGSSTIYYADTVKGRFTISRDNPKNTLFLQMTSLRSEDTAMYYCARYDYDDDYYAMEYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 17 - DNA, 705 bp, Mus sp.
  CDS (1)..(705)
atggcctggatttcacttatactctctctcctggctctcagctcaggggccatttcccaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggccagcccaagtcttcgccatcagtcaccctgtttccaccttcctctgaagagctcgagactaacaaggccacactggtgtgtacgatcactgatttctacccaggtgtggtgacagtggactggaaggtagatggtacccctgtcactcagggtatggagacaacccagccttccaaacagagcaacaacaagtacatggctagcagctacctgaccctgacagcaagagcatgggaaaggcatagcagttacagctgccaggtcactcatgaaggtcacactgtggagaagagtttgtcccgtgctgactgttcctag

SEQ ID NO: 18 - protein, 234 aa, Mus sp.
MAWISLILSLLALSSGAISQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 19 - DNA, 420 bp, Mus sp.
  CDS (1)..(420)
atggactccaggctcaatttagttttccttgtccttattttaaaaggtgtccagtgtgatgtgcagctggtggagtctgggggaggcttagtgcagcctggagggtcccggaaactctcctgtgcagcctctggattcactttcagtaactttggaatgcactgggttcgtcaggctccagagaagggactggagtgggtcgcatacattagtagtggcagtagtaccatctactatgcagacacagtgaagggccgattcaccatctccagagacaatcccaagaacaccctgttcctgcaaatgaccagtctaaggtctgaggacacggccatgtattactgtgcaagatatgattacgacgatgattactatgctatggaatactggggtcaaggaacctcagtcaccgtctcctca

SEQ ID NO: 20 - protein, 140 aa, Mus sp.
MDSRLNLVFLVLILKGVQCDVQLVESGGGLVQPGGSRKLSCAASGFTFSNFGMHWVRQAPEKGLEWVAYISSGSSTIYYADTVKGRFTISRDNPKNTLFLQMTSLRSEDTAMYYCARYDYDDDYYAMEYWGQGTSVTVSS

SEQ ID NO: 21 - DNA, 412 bp, Mus sp.
  CDS (29)..(412)
tgaaaagaatagacctggtttgtgaattatggcctggatttcacttatactctctctcctggctctcagctcaggggccatttcccaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtccta

SEQ ID NO: 22 - protein, 128 aa, Mus sp.
MAWISLILSLLALSSGAISQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVL

SEQ ID NO: 23 - protein, 5 aa, Mus sp.
NFGMH

SEQ ID NO: 24 - protein, 17 aa, Mus sp.
YISSGSSTIYYADTVKG

SEQ ID NO: 25 - protein, 12 aa, Mus sp.
YDYDDDYYAMEY

SEQ ID NO: 26 - protein, 14 aa, Mus sp.
RSSTGAVTTSNYAN

SEQ ID NO: 27 - protein, 7 aa, Mus sp.
GTNNRAP

SEQ ID NO: 28 - protein, 9 aa, Mus sp.
ALWYSNHWV

SEQ ID NO: 29 - DNA, 321 bp, Artificial
  Humanized sequence
cagtctgccctgactcagtccccagcaactctctctgtgtctcccggccaatctgtgagcctgtcctgtcgggcaagccagtctatttccaattatctgcactggtatcagcaacacccaggcgagagcccacggctcctgatcaaatacgcctctcaatccatttctggctccaacagacgcttctctggcagcggctccggcaccgatttcattctcagctttaatagcgttcaggcagaggattttgggatgtacttctgtcaacagagcaactcttggccttttaccttcggaagcgggactaaactgaccgttctg

SEQ ID NO: 30 - protein, 107 aa, Artificial
  Synthetic Construct
QSALTQSPATLSVSPGQSVSLSCRASQSISNYLHWYQQHPGESPRLLIKYASQSISGSNRRFSGSGSGTDFILSFNSVQAEDFGMYFCQQSNSWPFTFGSGTKLTVL

SEQ ID NO: 31 - DNA, 321 bp, Artificial
  Humanized sequence
caatctgcactgactcaatctccagcaacactgtccgtttctcctggacaatccgtgagcctcagctgtagggcatcccagtccattagcaactatctccactggtaccagcagcacccaggagaaagccctcgcctgctgataaaatacgcaagccagtctattagcggcagcaaccggcgcttcagcgggtccggctccgggacagactttatcctgtcttttagcagcgtgcaagccgaagactttggcatgtacttctgccagcagtctaacaattggccattcaccttcggatccggcacaaagctgaccgtgctg

SEQ ID NO: 32 - protein, 107 aa, Artificial
  Synthetic Construct
QSALTQSPATLSVSPGQSVSLSCRASQSISNYLHWYQQHPGESPRLLIKYASQSISGSNRRFSGSGSGTDFILSFSSVQAEDFGMYFCQQSNNWPFTFGSGTKLTVL

SEQ ID NO: 33 - DNA, 342 bp, Artificial
  Humanized sequence
caggttactctgaaagaatctggcccaactctggttaagcctacacagaccctgtccctcacctgttctttctccggctttagcctgagcacaagcggaatgggcgtcagctggatcagacaaccacccggcaagggcctggagtggctggcccacatctattgggatgatgataagaggtataacccttccctgaaatccagactcacaatttccaaggacacctccagcaaccaggtcttcctgaagataacctctgttgatccagtggacaccgcaacttactactgcgcccgcgactactatgccagctcttttgactactgggggcagggcacactc

SEQ ID NO: 34 - protein, 114 aa, Artificial
  Synthetic Construct
QVTLKESGPTLVKPTQTLSLTCSFSGFSLSTSGMGVSWIRQPPGKGLEWLAHIYWDDDKRYNPSLKSRLTISKDTSSNQVFLKITSVDPVDTATYYCARDYYASSFDYWGQGTL

SEQ ID NO: 35 - DNA, 342 bp, Artificial
  Humanized sequence
caggtgactctcaaagaatctggccctacactggtgaaacccactcagacactcagcctgacctgctccttcagcggcttctctctgagcacttccggactcggagtgagctggattcgccaacctcctgggaaaggcctggagtggctggcacacatatactgggatgacgacaaacgctataaccctagcctgaagtccagactcacaatctccaaagatacatccagcaatcaggtgtttctgaagatcacctccgtcgatccagtcgacaccgccacttactattgtgctcgcgattactatgccagctccttcgactactggggacagggaacactg

SEQ ID NO: 36 - protein, 114 aa, Artificial
  Synthetic Construct
QVTLKESGPTLVKPTQTLSLTCSFSGFSLSTSGLGVSWIRQPPGKGLEWLAHIYWDDDKRYNPSLKSRLTISKDTSSNQVFLKITSVDPVDTATYYCARDYYASSFDYWGQGTL

SEQ ID NO: 37 - DNA, 342 bp, Artificial
  Humanized sequence
caggtcacactcaaagaatctggaccaacactcgttaaaccaactcaaacactcagcctgacttgcagcttttctggcttcagcctctccactagcggcctcggcgtgtcctggatccggcagccccctggaaaaggcctggagtggctggcccacatctattgggacgatgacaagcggtacaatccaagcctgaaatctcggctgaccatatctaaggatacttcttccaatcaggtgttcctgaagatcacatctgtggctcctgtcgacactgccacttattattgcgcaagggattactacgcatcctccttcgattactggggccagggaactctg

SEQ ID NO: 38 - protein, 114 aa, Artificial
  Synthetic Construct
QVTLKESGPTLVKPTQTLSLTCSFSGFSLSTSGLGVSWIRQPPGKGLEWLAHIYWDDDKRYNPSLKSRLTISKDTSSNQVFLKITSVAPVDTATYYCARDYYASSFDYWGQGTL

SEQ ID NO: 39 - DNA, 1059 bp, Homo sapiens
  CDS (1)..(1059)
ctggaggcctctgaggaagtggagcttgagccctgcctggctcccagcctggagcagcaagagcaggagctgacagtagcccttgggcagcctgtgcggctgtgctgtgggcgggctgagcgtggtggccactggtacaaggagggcagtcgcctggcacctgctggccgtgtacggggctggaggggccgcctagagattgccagcttcctacctgaggatgctggccgctacctctgcctggcacgaggctccatgatcgtcctgcagaatctcaccttgattacaggtgactccttgacctccagcaacgatgatgaggaccccaagtcccatagggacctctcgaataggcacagttacccccagcaagcaccctactggacacacccccagcgcatggagaagaaactgcatgcagtacctgcggggaacaccgtcaagttccgctgtccagctgcaggcaaccccacgcccaccatccgctggcttaaggatggacaggcctttcatggggagaaccgcattggaggcattcggctgcgccatcagcactggagtctcgtgatggagagcgtggtgccctcggaccgcggcacatacacctgcctggtagagaacgctgtgggcagcatccgttataactacctgctagatgtgctggagcggtccccgcaccggcccatcctgcaggccgggctcccggccaacaccacagccgtggtgggcagcgacgtggagctgctgtgcaaggtgtacagcgatgcccagccccacatccagtggctgaagcacatcgtcatcaacggcagcagcttcggagccgacggtttcccctatgtgcaagtcctaaagactgcagacatcaatagctcagaggtggaggtcctgtacctgcggaacgtgtcagccgaggacgcaggcgagtacacctgcctcgcaggcaattccatcggcctctcctaccagtctgcctggctcacggtgctgccagaggaggaccccacatggaccgcagcagcgcccgaggccgctagccatcaccatcatcatcactga

SEQ ID NO: 40 - protein, 352 aa, Homo sapiens
LEASEEVELEPCLAPSLEQQEQELTVALGQPVRLCCGRAERGGHWYKEGSRLAPAGRVRGWRGRLEIASFLPEDAGRYLCLARGSMIVLQNLTLITGDSLTSSNDDEDPKSHRDLSNRHSYPQQAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLERSPHRPILQAGLPANTTAVVGSDVELLCKVYSDAQPHIQWLKHIVINGSSFGADGFPYVQVLKTADINSSEVEVLYLRNVSAEDAGEYTCLAGNSIGLSYQSAWLTVLPEEDPTWTAAAPEAASHHHHHH

SEQ ID NO: 41 - DNA, 726 bp, Homo sapiens
  CDS (1)..(726)
ctggaggcctctgaggaagtggagcttgagccctgccaagcaccctactggacacacccccagcgcatggagaagaaactgcatgcagtacctgcggggaacaccgtcaagttccgctgtccagctgcaggcaaccccacgcccaccatccgctggcttaaggatggacaggcctttcatggggagaaccgcattggaggcattcggctgcgccatcagcactggagtctcgtgatggagagcgtggtgccctcggaccgcggcacatacacctgcctggtagagaacgctgtgggcagcatccgttataactacctgctagatgtgctggagcggtccccgcaccggcccatcctgcaggccgggctcccggccaacaccacagccgtggtgggcagcgacgtggagctgctgtgcaaggtgtacagcgatgcccagccccacatccagtggctgaagcacatcgtcatcaacggcagcagcttcggagccgacggtttcccctatgtgcaagtcctaaagactgcagacatcaatagctcagaggtggaggtcctgtacctgcggaacgtgtcagccgaggacgcaggcgagtacacctgcctcgcaggcaattccatcggcctctcctaccagtctgcctggctcacggtgctgccagaggaggaccccacatggaccgcagcagcgcccgaggccgctagccatcaccatcatcatcactga

SEQ ID NO: 42 - protein, 241 aa, Homo sapiens
LEASEEVELEPCQAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLERSPHRPILQAGLPANTTAVVGSDVELLCKVYSDAQPHIQWLKHIVINGSSFGADGFPYVQVLKTADINSSEVEVLYLRNVSAEDAGEYTCLAGNSIGLSYQSAWLTVLPEEDPTWTAAAPEAASHHHHHH

SEQ ID NO: 43 - DNA, 1059 bp, Mus sp.
  CDS (1)..(1059)
cttgaggcctctgaggaaatggagcaggagccctgcctagccccaatcctggagcagcaagagcaggtgttgacggtggccctggggcagcctgtgaggctgtgctgtgggcgcaccgagcgtggtcgtcactggtacaaagagggcagccgcctagcatctgctgggcgagtacggggttggagaggccgcctggagatcgccagcttccttcctgaggatgctggccgatacctctgcctggcccgtggctccatgaccgtcgtacacaatcttacgttgcttatggatgactccttaacctccatcagtaatgatgaagaccccaagacactcagcagctcctcgagtggtcatgtctacccacagcaagcaccctactggacacacccccaacgcatggagaagaaactgcatgcagtgcctgccgggaatactgtcaaattccgctgtccagctgcagggaaccccatgcctaccatccactggctcaaggatggacaggccttccacggggagaatcgtattggaggcattcggctgcgccaccaacactggagcctggtgatggaaagtgtggtaccctcggaccgtggcacatacacatgccttgtggagaactctctgggtagcattcgctacagctatctcctggatgtgctggagcggtccccgcaccggcccatcctgcaggcggggctcccagccaacaccacagctgtggtgggcagcgacgtggagctactctgcaaggtgtacagcgacgcccagccccacatacagtggctgaaacacgtcgtcatcaacggcagcagcttcggcgccgacggtttcccctacgtacaagtcctgaagacaacagacatcaatagctcggaggtagaggtcttgtatctgaggaacgtgtccgctgaggatgcaggagagtatacctgtctggcgggcaactccatcggcctttcctaccagtcagcgtggctcacggtgctgccagaggaagacctcacgtggacaacagcaacccctgaggccgctagccatcaccatcatcatcactga

SEQ ID NO: 44 - protein, 352 aa, Mus sp.
LEASEEMEQEPCLAPILEQQEQVLTVALGQPVRLCCGRTERGRHWYKEGSRLASAGRVRGWRGRLEIASFLPEDAGRYLCLARGSMTVVHNLTLLMDDSLTSISNDEDPKTLSSSSSGHVYPQQAPYWTHPQRMEKKLHAVPAGNTVKFRCPAAGNPMPTIHWLKDGQAFHGENRIGGIRLRHQHWSLVMESVVPSDRGTYTCLVENSLGSIRYSYLLDVLERSPHRPILQAGLPANTTAVVGSDVELLCKVYSDAQPHIQWLKHVVINGSSFGADGFPYVQVLKTTDINSSEVEVLYLRNVSAEDAGEYTCLAGNSIGLSYQSAWLTVLPEEDLTWTTATPEAASHHHHHH

SEQ ID NO: 45 - DNA, 1773 bp, Homo sapiens
  CDS (1)..(1773)
aggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatagcgctagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 46 - protein, 590 aa, Homo sapiens
RPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIALEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - DNA, 1773 bp, Homo sapiens
  CDS (1)..(1773)
cggccctccttcagtttagttgaggataccacattagagccagaagagccaccaaccaaataccaaatctctcaaccagaagtgtacgtggctgcaccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtggtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctgtcagcgctagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 48 - protein, 590 aa, Homo sapiens
RPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLSALEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - DNA, 2403 bp, Rattus sp.
  CDS (1)..(2403)
atgtggctgctgttggctttgttgagcatctttcaggagacaccagccttctcccttgaggcctctgaggaaatggaacaggagccctgcccagccccaatctcggagcagcaagagcaggtgttgactgtggcccttgggcagcctgtgcggctatgctgtggccgcactgagcgtggtcgtcattggtacaaggagggcagccgtttagcatctgctgggagagtacggggctggagaggccgcctggagatcgccagcttccttcctgaggatgctggccggtacctctgcctggcccgtggctccatgactgtcgtacacaatcttactttgattatggatgactccttaccctccatcaataacgaggaccccaagaccctcagcagctcctcgagtgggcactcctacctgcagcaagcaccttactggacacacccccaacgcatggagaagaaactgcacgcggtacctgccgggaacactgtcaaattccgctgtccagctgcagggaaccccatgcccaccatccactggctcaagaacggacaggccttccacggagagaatcgtatcggaggcattcggctgcgtcaccaacactggagcctcgtgatggagagcgtggtgccctcagaccgtggcacgtacacgtgtcttgtggagaactctctgggtagcattcgctacagctatctgctggatgtgctggagcggtccccgcaccggcccatcctgcaggcgggactcccagccaacaccacggctgtggttggcagcaacgtggagctgctgtgcaaggtgtacagtgacgcccagccgcacatccagtggctgaagcacatcgttatcaacggcagcagtttcggcgctgatggtttcccctacgtacaagtcctgaagacaacagacatcaatagctcagaggtggaggtgctgtatctgaggaacgtgtcggctgaggatgcaggggagtacacctgcctggcgggcaactccatcggcctctcctaccagtcagcgtggctcacagtgctacccgcagaggaagaagacctcgcgtggacaacagcaacatcggaggccagatatacagatattatcctatatgtatctggctcactggctttggttttgctcctgctgctggccggggtgtatcaccgacaagcaatccacggccaccactctcgacagcctgtcactgtacagaagctgtcccggttccctttggcccggcagttctccttggagtcgaggtcctctggcaagtcaagtttgtccctggtgcgaggtgtccggctctcctccagtggcccgcccttgctcacgggccttgtgagtctagacctacctctcgatccactttgggagttcccccgggacaggctggtgctcggaaagcccctgggtgagggctgctttgggcaagtggttcgtgcagaagcccttggcatggattcctcccggccagaccaaaccagcaccgtggctgtgaagatgctgaaagacaatgcctccgacaaggatttggcagacctgatctctgagatggagatgatgaagctaatcggaagacacaagaacatcattaacctgctgggtgtctgcactcaggaagggcccctgtatgtgattgtggaatatgcggccaagggaaaccttcgggaattcctccgtgcccggcgtcccccaggccctgatctcagccctgatgggcctcggagcagcgaaggaccgctctccttcccggccctggtctcctgtgcctaccaggtggcccgaggcatgcagtatctggagtctcggaagtgcatccaccgggacctggctgcccgaaacgtgctggtgaccgaggatgacgtgatgaagatcgctgactttggtctggcccgtggtgtccaccacatcgactactataagaaaaccagcaatggccgcctgccagtcaagtggatggctcctgaggcgttgtttgaccgtgtatacacacaccagagtgacgtgtggtccttcgggatcctgctgtgggaaatcttcaccctcgggggctccccataccccggcatcccagtggaggagctgttctcactgctgcgagaggggcacaggatggagcggcccccaaactgcccctcagagctgtatgggctaatgagggagtgttggcacgcagctccttctcagaggccgacttttaagcagctggtggaagctctggacaaggtcctgctggctgtctctgaagagtaccttgacctccgcctgacctttggaccctattcccccaacaatggggatgccagcagcacgtgctcctccagcgactcggttttcagccacgaccctttgcccctcgagccaagccccttcccatttcctgaggcgcagaccacatga

SEQ ID NO: 50 - protein, 800 aa, Rattus sp.
MWLLLALLSIFQETPAFSLEASEEMEQEPCPAPISEQQEQVLTVALGQPVRLCCGRTERGRHWYKEGSRLASAGRVRGWRGRLEIASFLPEDAGRYLCLARGSMTVVHNLTLIMDDSLPSINNEDPKTLSSSSSGHSYLQQAPYWTHPQRMEKKLHAVPAGNTVKFRCPAAGNPMPTIHWLKNGQAFHGENRIGGIRLRHQHWSLVMESVVPSDRGTYTCLVENSLGSIRYSYLLDVLERSPHRPILQAGLPANTTAVVGSNVELLCKVYSDAQPHIQWLKHIVINGSSFGADGFPYVQVLKTTDINSSEVEVLYLRNVSAEDAGEYTCLAGNSIGLSYQSAWLTVLPAEEEDLAWTTATSEARYTDIILYVSGSLALVLLLLLAGVYHRQAIHGHHSRQPVTVQKLSRFPLARQFSLESRSSGKSSLSLVRGVRLSSSGPPLLTGLVSLDLPLDPLWEFPRDRLVLGKPLGEGCFGQVVRAEALGMDSSRPDQTSTVAVKMLKDNASDKDLADLISEMEMMKLIGRHKNIINLLGVCTQEGPLYVIVEYAAKGNLREFLRARRPPGPDLSPDGPRSSEGPLSFPALVSCAYQVARGMQYLESRKCIHRDLAARNVLVTEDDVMKIADFGLARGVHHIDYYKKTSNGRLPVKWMAPEALFDRVYTHQSDVWSFGILLWEIFTLGGSPYPGIPVEELFSLLREGHRMERPPNCPSELYGLMRECWHAAPSQRPTFKQLVEALDKVLLAVSEEYLDLRLTFGPYSPNNGDASSTCSSSDSVFSHDPLPLEPSPFPFPEAQTT

SEQ ID NO: 51 - DNA, 31 bp, Mus sp.
cggccgccaggtacctggaattcggcttggg

SEQ ID NO: 52 - DNA, 31 bp, Mus sp.
ctagaaggcactctagaggctgatctcgagc

SEQ ID NO: 53 - DNA, 14 bp, Mus sp.
cgagctctgtacaa

SEQ ID NO: 54 - DNA, 22 bp, Mus sp.
ctagttgtacagagctcggtac

SEQ ID NO: 55 - DNA, 32 bp, Mus sp.
gcgccgtcctggagctcctgctgctgaggtgg

SEQ ID NO: 56 - DNA, 24 bp, Mus sp.
ggcaactagaaggcacagtcgagg

SEQ ID NO: 57 - DNA, 27 bp, Mus sp.
gtccctgggatcctcgaggaagtggag

SEQ ID NO: 58 - DNA, 31 bp, Mus sp.
gcgcctgcccgagctccagcccggccagcag

SEQ ID NO: 59 - protein, 19 aa, Mus sp.
LALAVLLLAGLELLLLRWQ

SEQ ID NO: 60 - protein, 12 aa, Mus sp.
LALAVLLLLRWQ

SEQ ID NO: 61 - DNA, 52 bp, Mus sp.
ggccttggctgtgctcctgctgctgaggtggcagtttcctgcacactacagg

SEQ ID NO: 62 - DNA, 52 bp, Mus sp.
cctgtagtgtgcaggaaactgccacctcagcagcaggagcacagccaaggcc

SEQ ID NO: 63 - DNA, 23 bp, Mus sp.
cgactggagcacgaggacactga

SEQ ID NO: 64 - DNA, 21 bp, Mus sp.
tatgcaaggcttacaaccaca

SEQ ID NO: 65 - DNA, 24 bp, Mus sp.
ctcattcctgttgaagctcttgac

SEQ ID NO: 66 - DNA, 27 bp, Mus sp.
acactcagcacgggacaaactcttctc

SEQ ID NO: 67 - DNA, 27 bp, Mus sp.
acactctgcaggagacagactcttttc

SEQ ID NO: 68 - DNA, 1062 bp, Homo sapiens
  CDS (1)..(1062)
ctggaggcctctgaggaagtggagcttgagccctgcctggctcccagcctggagcagcaagagcaggagctgacagtagcccttgggcagcctgtgcggctgtgctgtgggcgggctgagcgtggtggccactggtacaaggagggcagtcgcctggcacctgctggccgtgtacggggctggaggggccgcctagagattgccagcttcctacctgaggatgctggccgctacctctgcctggcacgaggctccatgatcgtcctgcagaatctcaccttgattacaggtgactccttgacctccagcaacgatgatgaggaccccaagtcccatagggacctctcgaataggcacagttacccccagcaagcaccctactggacacacccccagcgcatggagaagaaactgcatgcagtacctgcggggaacaccgtcaagttccgctgtccagctgcaggcaaccccacgcccaccatccgctggcttaaggatggacaggcctttcatggggagaaccgcattggaggcattcggctgcgccatcagcactggagtctcgtgatggagagcgtggtgccctcggaccgcggcacatacacctgcctggtagagaacgctgtgggcagcatccgttataactacctgctagatgtgctggagcggtccccgcaccggcccatcctgcaggccgggctcccggccaacaccacagccgtggtgggcagcgacgtggagctgctgtgcaaggtgtacagcgatgcccagccccacatccagtggctgaagcacatcgtcatcaacggcagcagcttcggagccgacggtttcccctatgtgcaagtcctaaagactgcagacatcaatagctcagaggtggaggtcctgtacctgcggaacgtgtcagccgaggacgcaggcgagtacacctgcctcgcaggcaattccatcggcctctcctaccagtctgcctggctcacggtgccagaggaggaccccacatggaccgcagcagcgcccgaggccgctagctggagccacccgcagttcgaaaaatga

SEQ ID NO: 69 - protein, 353 aa, Homo sapiens
LEASEEVELEPCLAPSLEQQEQELTVALGQPVRLCCGRAERGGHWYKEGSRLAPAGRVRGWRGRLEIASFLPEDAGRYLCLARGSMIVLQNLTLITGDSLTSSNDDEDPKSHRDLSNRHSYPQQAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLERSPHRPILQAGLPANTTAVVGSDVELLCKVYSDAQPHIQWLKHIVINGSSFGADGFPYVQVLKTADINSSEVEVLYLRNVSAEDAGEYTCLAGNSIGLSYQSAWLTVPEEDPTWTAAAPEAASWSHPQFEK

SEQ ID NO: 70 - protein, 98 aa, Homo sapiens
QAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVL

SEQ ID NO: 71 - DNA, 648 bp, Homo sapiens
  CDS (1)..(648)
cagagcgaattgactcagccaccctcagcgtctgggacccccgggcagagggtcaccatctcttgttctggaggctactccaacatgggaagcaattatgcacactggtaccagcagctcccaggaacggcccccaaactcctcatctataacaataatcagcggccctcaggggtccctgaccgattctctggctccaagtctggcacctcagcctccctggccatcagtgggctccggtccgaggatgaggctgattattactgtgcagcatgggatgacagcctgagtggtccggtgttcggcggagggaccaagctgaccgtcctaggtcagcccaaggctgccccctcggtcactctgttcccgccctcctctgaggagcttcaagccaacaaggccacactggtgtgtctgatcagtgacttctacccgggagctgtgacagtggcctggaaggcagatggcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 72 - protein, 216 aa, Homo sapiens
QSELTQPPSASGTPGQRVTISCSGGYSNMGSNYAHWYQQLPGTAPKLLIYNNNQRPSGVPDRFSGSKSGTSASLAISGLRSEDEADYYCAAWDDSLSGPVFGGGTKLTVLGQPKAAPSVTLFPPSSEELQANKATLVCLISDFYPGAVTVAWKADGSPVKAGVETTTPSKQSNNKYAASSYLSLTPEQWKSHRSYSCQVTHEGSTVEKTVAPTECS

SEQ ID NO: 73 - protein, 9 aa, Homo sapiens
SNMGSNYAH

SEQ ID NO: 74 - protein, 5 aa, Homo sapiens
YNNNQ

SEQ ID NO: 75 - protein, 8 aa, Homo sapiens
AWDDSLSG

SEQ ID NO: 76 - DNA, 411 bp, Homo sapiens
  CDS (1)..(411)
gaagttcaattgttagagtctggtggcggtcttgttcagcctggtggttctttacgtctttcttgcgctgcttccggattcactttctctccttactggatgggttgggttcgccaagctcctggtaaaggtttggagtgggtttctgttatctggccttctggtggcactactgattatgctgactccgttaaaggtcgcttcactatctctagagacaactctaagaatactctctacttgcagatgaacagcttaagggctgaggacacggccgtgtattactgtgcgagagtccatccagctggccacaacgaccactggggccagggaaccctggtcaccgtctcaagcgcctccaccaagggcccatcggtcttcccgctagcaccctcctccaagagcacctct

SEQ ID NO: 77 - protein, 137 aa, Homo sapiens
EVQLLESGGGLVQPGGSLRLSCAASGFTFSPYWMGWVRQAPGKGLEWVSVIWPSGGTTDYADSVKGRFTISRDNSKNTLYLQMNSLRAEDTAVYYCARVHPAGHNDHWGQGTLVTVSSASTKGPSVFPLAPSSKSTS

SEQ ID NO: 78 - protein, 9 aa, Homo sapiens
FTFSPYWMG

SEQ ID NO: 79 - protein, 10 aa, Homo sapiens
VIWPSGGTTD

SEQ ID NO: 80 - protein, 10 aa, Homo sapiens
RVHPAGHNDH

SEQ ID NO: 81 - DNA, 645 bp, Homo sapiens
  CDS (1)..(645)
caagacatccagatgacccagtctccatcctccctgtctttgtctccaggggaaagagccaccctctcctgcagggccagtcagagtattaacagaaacttaggctggtaccagcagaagcctggccaggctcccaggctcctcatctatggtgcgtccaccagggccactggtatcccagccaggttcactggcagtgggtctgggacagagttctctctcaccatcagcagcctgcagtctgaagattatgcaatttattattgtcagcagtatgataactggccgatcatcttcggccaagggacacgactggagattaaaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 82 - protein, 215 aa, Homo sapiens
QDIQMTQSPSSLSLSPGERATLSCRASQSINRNLGWYQQKPGQAPRLLIYGASTRATGIPARFTGSGSGTEFSLTISSLQSEDYAIYYCQQYDNWPIIFGQGTRLEIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 7 aa, Homo sapiens
SINRNLG

SEQ ID NO: 84 - protein, 5 aa, Homo sapiens
YGAST

SEQ ID NO: 85 - protein, 6 aa, Homo sapiens
QYDNWP

SEQ ID NO: 86 - DNA, 411 bp, Homo sapiens
  CDS (1)..(411)
gaagttcaattgttagagtctggtggcggtcttgttcagcctggtggttctttacgtctttcttgcgctgcttccggattcactttctctcgttactttatgaagtgggttcgccaagctcctggtaaaggtttggagtgggtttcttggatctctccttctggtggccttactgagtatgctgactccgttaaaggtcgcttcactatctctagagacaactctaagaatactctctacttgcagatgaacagcttaagggctgaggacacggccgtgtattactgtgccagagcctcatacggtgcctggctggactactggggccagggcaccctggtcaccgtctcaagcgcctccaccaagggcccatcggtcttcccgctagcaccctcctccaagagcacctct

SEQ ID NO: 87 - protein, 137 aa, Homo sapiens
EVQLLESGGGLVQPGGSLRLSCAASGFTFSRYFMKWVRQAPGKGLEWVSWISPSGGLTEYADSVKGRFTISRDNSKNTLYLQMNSLRAEDTAVYYCARASYGAWLDYWGQGTLVTVSSASTKGPSVFPLAPSSKSTS

SEQ ID NO: 88 - protein, 9 aa, Homo sapiens
FTFSRYFMK

SEQ ID NO: 89 - protein, 10 aa, Homo sapiens
WISPSGGLTE

SEQ ID NO: 90 - protein, 10 aa, Homo sapiens
RASYGAWLDY

SEQ ID NO: 91 - DNA, 651 bp, Homo sapiens
  CDS (1)..(651)
cagagcgtcttgactcagccaccctcagcgtctgggacccccggccagggggtcaccatctcttgttctggaagcagctccaacatcggaagtaatagtgttaactggtaccagcagctcccaggagcggcccccaaactcctcatctatagtaacaatctgcggccctcaggggtccctgcccgattctctggctccaagtctggcacctcagcctccctggccatcagtggcctccggtctgaggatgaggctgattattactgtgcagcctgggatgacggcctggatgcctatgtggtgttcggcggagggaccaaactgaccgtcctgggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctgcagaatgctct

SEQ ID NO: 92 - protein, 217 aa, Homo sapiens
QSVLTQPPSASGTPGQGVTISCSGSSSNIGSNSVNWYQQLPGAAPKLLIYSNNLRPSGVPARFSGSKSGTSASLAISGLRSEDEADYYCAAWDDGLDAYVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPAECS

SEQ ID NO: 93 - protein, 9 aa, Homo sapiens
SNIGSNSVN

SEQ ID NO: 94 - protein, 5 aa, Homo sapiens
YSNNL

SEQ ID NO: 95 - protein, 9 aa, Homo sapiens
AWDDGLDAY

SEQ ID NO: 96 - DNA, 414 bp, Homo sapiens
  CDS (1)..(414)
gaagttcaattgttagagtctggtggcggtcttgttcagcctggtggttctttacgtctttcttgcgctgcttccggattcactttctctacttactggatggcttgggttcgccaagctcctggtaaaggtttggagtgggtttctgttatcggtccttctggtggcatgactgtttatgctgactccgttaaaggtcgcttcactatctctagagacaactctaagaatactctctacttgcagatgaacagcttaagggctgaggacacggccgtgtattactgtgcacggattaatagtgggagctcccactttgactactggggccagggaaccctggtcaccgtctcaagcgcctccaccaagggcccatcggtcttcccgctagcaccctcctccaagagcacctct

SEQ ID NO: 97 - protein, 138 aa, Homo sapiens
EVQLLESGGGLVQPGGSLRLSCAASGFTFSTYWMAWVRQAPGKGLEWVSVIGPSGGMTVYADSVKGRFTISRDNSKNTLYLQMNSLRAEDTAVYYCARINSGSSHFDYWGQGTLVTVSSASTKGPSVFPLAPSSKSTS

SEQ ID NO: 98 - protein, 9 aa, Homo sapiens
FTFSTYWMA

SEQ ID NO: 99 - protein, 10 aa, Homo sapiens
VIGPSGGMTV

SEQ ID NO: 100 - protein, 11 aa, Homo sapiens
RINSGSSHFDY

SEQ ID NO: 101 - DNA, 651 bp, Homo sapiens
  CDS (1)..(651)
cagtacgaattgactcagccaccctcagcgtctgggacccccgggcagagggtcaccatctcttgttctggaagcagctccaacatcggaagcaatattgtacactggtaccagcagctcccaggaacggcccccaaactcctcatatatggtaataatcagcggccctcaggggtccctgaccgattctctgtctccaagtctggctcctcagcctccctggccatcagtgggctccagtctgaggatgaggctgattattactgtgcatcatgggatgacagcctgaaagccttttatgtcttcggaactgggaccaaggtcaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 102 - protein, 217 aa, Homo sapiens
QYELTQPPSASGTPGQRVTISCSGSSSNIGSNIVHWYQQLPGTAPKLLIYGNNQRPSGVPDRFSVSKSGSSASLAISGLQSEDEADYYCASWDDSLKAFYVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 103 - protein, 9 aa, Homo sapiens
SNIGSNIVH

SEQ ID NO: 104 - protein, 5 aa, Homo sapiens
YGNNQ

SEQ ID NO: 105 - protein, 9 aa, Homo sapiens
SWDDSLKAF

SEQ ID NO: 106 - DNA, 420 bp, Homo sapiens
  CDS (1)..(420)
gaagttcaattgttagagtctggtggcggtcttgttcagcctggtggttctttacgtctttcttgcgctgcttccggattcactttctctcattactggatgggttgggttcgccaagctcctggtaaaggtttggagtgggtttctgttatctctccttctggtggccttactatttatgctgactccgttaaaggtcgcttcactatctctagagacaactctaagaatactctctacttgcagatgaacagcttaagggctgaggacacggccgtgtattactgtgcacggagcagtggctgggctctggatgcttttgatatctggggccaagggacaatggtcaccgtctcaagcgcctccaccaagggcccatcggtcttcccgctagcaccctcctccaagagcacctctggg

SEQ ID NO: 107 - protein, 140 aa, Homo sapiens
EVQLLESGGGLVQPGGSLRLSCAASGFTFSHYWMGWVRQAPGKGLEWVSVISPSGGLTIYADSVKGRFTISRDNSKNTLYLQMNSLRAEDTAVYYCARSSGWALDAFDIWGQGTMVTVSSASTKGPSVFPLAPSSKSTSG

SEQ ID NO: 108 - protein, 9 aa, Homo sapiens
FTFSHYWMG

SEQ ID NO: 109 - protein, 10 aa, Homo sapiens
VISPSGGLTI

SEQ ID NO: 110 - protein, 12 aa, Homo sapiens
RSSGWALDAFDI

SEQ ID NO: 111 - DNA, 1215 bp, Homo sapiens
  CDS (1)..(1215)
atgcggctgctgctggccctgttgggggtcctgctgagtgtgcctgggcctccagtcttgtccctggaggcctctgaggaagtggagcttgagccctgcctggctcccagcctggagcagcaagagcaggagctgacagtagcccttgggcagcctgtgcggctgtgctgtgggcgggctgagcgtggtggccactggtacaaggagggcagtcgcctggcacctgctggccgtgtacggggctggaggggccgcctagagattgccagcttcctacctgaggatgctggccgctacctctgcctggcacgaggctccatgatcgtcctgcagaatctcaccttgattacaggtgactccttgacctccagcaacgatgatgaggaccccaagtcccatagggacctctcgaataggcacagttacccccagcaagcaccctactggacacacccccagcgcatggagaagaaactgcatgcagtacctgcggggaacaccgtcaagttccgctgtccagctgcaggcaaccccacgaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttga

SEQ ID NO: 112 - protein, 404 aa, Homo sapiens
MRLLLALLGVLLSVPGPPVLSLEASEEVELEPCLAPSLEQQEQELTVALGQPVRLCCGRAERGGHWYKEGSRLAPAGRVRGWRGRLEIASFLPEDAGRYLCLARGSMIVLQNLTLITGDSLTSSNDDEDPKSHRDLSNRHSYPQQAPYWTHPQRMEKKLHAVPAGNTVKFRCPAAGNPTKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - DNA, 1068 bp, Homo sapiens
  CDS (1)..(1068)
atgcggctgctgctggccctgttgggggtcctgctgagtgtgcctgggcctccagtcttgtccctggaggcctctgaggaagtggagcttgagccccaagcaccctactggacacacccccagcgcatggagaagaaactgcatgcagtacctgcggggaacaccgtcaagttccgctgtccagctgcaggcaaccccacgcccaccatccgctggcttaaggatggacaggcctttcatggggagaaccgcattggaggcattcggctgcgccatcagcactggagtctcgtgatggagagcgtggtgccctcggaccgcggcacatacacctgcctggtagagaacgctgtgggcagcatccgttataactacctgctagatgtgctgaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttga

SEQ ID NO: 114 - protein, 355 aa, Homo sapiens
MRLLLALLGVLLSVPGPPVLSLEASEEVELEPQAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - DNA, 1188 bp, Homo sapiens
  CDS (1)..(1188)
atgcggctgctgctggccctgttgggggtcctgctgagtgtgcctgggcctccagtcttgtccctggaggcctctgaggaagtggagcttgagcccaacgctgtgggcagcatccgttataactacctgctagatgtgctggagcggtccccgcaccggcccatcctgcaggccgggctcccggccaacaccacagccgtggtgggcagcgacgtggagctgctgtgcaaggtgtacagcgatgcccagccccacatccagtggctgaagcacatcgtcatcaacggcagcagcttcggagccgacggtttcccctatgtgcaagtcctaaagactgcagacatcaatagctcagaggtggaggtcctgtacctgcggaacgtgtcagccgaggacgcaggcgagtacacctgcctcgcaggcaattccatcggcctctcctaccagtctgcctggctcacggtgctgccagaggaggaccccacatggaccgcagcagcgcccgaggccaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttga

SEQ ID NO: 116 - protein, 395 aa, Homo sapiens
MRLLLALLGVLLSVPGPPVLSLEASEEVELEPNAVGSIRYNYLLDVLERSPHRPILQAGLPANTTAVVGSDVELLCKVYSDAQPHIQWLKHIVINGSSFGADGFPYVQVLKTADINSSEVEVLYLRNVSAEDAGEYTCLAGNSIGLSYQSAWLTVLPEEDPTWTAAAPEAKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 117 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2011/0150903 A1
(43) Pub. Date: Jun. 23, 2011

(54) FGF-R4 RECEPTOR-SPECIFIC ANTAGONISTS

(75) Inventors: Nicolas Baurin, Paris (FR); Pierre-Francois Berne, Paris (FR); Francis Blanche, Paris (FR); Francoise Bono, Paris (FR); Beatrice Cameron, Paris (FR); Tarik Dabdoubi, Paris (FR); Corentin Herbert, Paris (FR); Vincent Mikol, Paris (FR); Elisabeth Remy, Paris (FR)

(73) Assignee: SANOFI-AVENTIS, Paris (FR)

(21) Appl. No.: 13/002,845

(22) PCT Filed: Jul. 7, 2009
(86) PCT No.: PCT/FR2009/051343

§ 371 Date: Feb. 25, 2011

(30) Foreign Application Priority Data
Jul. 8, 2008 (FR) 08/03888

(51) Int. Cl. A61K 39/395; C07K 16/22; C12N 5/07; C12P 21/00; C07H 21/04; C12N 15/63; A61P 35/00

(52) U.S. Cl. 424/158.1; 530/389.2; 530/387.3; 530/391.7; 435/332; 435/71.1; 536/23.53; 435/320.1

(57) ABSTRACT

The present invention relates to FGF-R4 receptor-specific antagonist molecules enabling the inhibition of the activity of said receptor. Said antagonists are, particularly, FGF-R4-specific antibodies enabling the inhibition of the activity of said receptor. The present invention also relates to the therapeutic use of said antibodies, particularly in the field of angiogenesis and in the treatment of certain types of cancer.

The subject of the present invention is antagonists specific for the FGF receptor 4 (FGF-R4) which make it possible to inhibit the activity of this receptor. These antagonists are in particular antibodies directed specifically against the FGF receptor 4 (FGF-R4).

A subject of the present invention is also the therapeutic use of these antagonists, in particular in the angiogenesis field and in the treatment of certain types of cancer.

FGFs (Fibroblast Growth Factors) are among the first molecules described as being capable of stimulating vascular cell proliferation, migration and differentiation in vitro and in vivo. An abundant literature describes the induction of angiogenesis and the formation of blood capillaries in vitro and in vivo by FGFs. FGFs are also involved in tumour angiogenesis by promoting the formation of blood vessels recruited by the tumour.

The human FGF family is composed of at least 23 members which all have a conserved central domain of 120 amino acids. They exert their biological activity by interacting with their high affinity receptors of tyrosine-kinase type (FGF-R) and heparan sulphate proteoglycans, which are components present on most cell surfaces and extracellular matrices (low-affinity binding site), so as to form a ternary complex. Some FGFs have a high affinity for several FGF-Rs, whereas others activate specifically one receptor or one isoform of a receptor.

An FGF-R4-specific ligand has been identified by Xie and al (Cytokine, 1999, 11:729-35.) This ligand, called FGF19, is a ligand with high affinity for FGF-R4 exclusively, and the binding of which to the receptor is heparin-dependent or heparan sulphate-dependent. FGF19 has been identified in adult animals, only in the hepatocytes and the small intestine where it regulates the synthesis of bile acid by the liver. It appears to be a growth factor involved during embryonic development, and appears to be involved in foetal brain development in the zebra fish and humans.

Other FGF-R4 ligands are described, such as FGF1 or FGF2. These ligands strongly activate FGF-R4, but are not specific of FGF-R4: they also bind to other FGF-Rs (Ornitz and al., J. Biol. Chem., 1996, 271:15292-7).

The activation of the FGF-R4 receptor results in several types of cell signalling. Among these, the most conventional form corresponds to the setting up of a phosphorylation cascade-mediated signalling pathway subsequent to stimulation of FGF-R4 by FGF. This induction results in the autophosphorylation of the tyrosine kinase domain of FGF-R4 and serves to initiate an intracellular signalling pathway dependent on the phosphorylation of other signalling proteins such as AKT, p44/42, JNK etc. This phosphorylation-mediated signalling varies according to the cell type and according to the coreceptors or the adhesion molecules present at the surface of these cells (Cavallaro and al., Nat. Cell Biol. 3/7, 650-657 (2001); Stadler and al., Cell. Signal. 18/6, 783-794. (2006); Lin and al., J Biol Chem., 2007; 14:27277-84. (2007)). Another method of signalling that is important for FGF-Rs, including FGF-R4, is the internalization of the receptor after activation in combination with its ligand. This mechanism is not dependent on the tyrosine kinase activity of the receptor, but on a short C-terminal sequence of FGF-R4 (Klingenberg and al., J. Cell Sci., 113/Pt10:1827-1838 (2000)).

Four distinct forms of FGF-R4 are described in the literature. A full-length form with 2 polymorphic variants at position 388, namely FGF-R4 Gly388, which is the normal form of the receptor, and the Arg388 form which is described in the context of several tumours (Bange and al., Cancer Res. 62/3, 840-847 (2002); Spinola and al., J Clin Oncol 23, 7307-7311 (2005); Stadler and al., Cell. Signal. 18/6, 783-794. (2006)). A soluble form, which is expressed in mammary tumour cells, has also been discovered (Takaishi and al., Biochem Biophys Res Commun., 2000, 267:658-62). A fourth form, which is truncated in the extracellular portion, has been described in certain hypophyseal adenomas (Ezzat and al., J Clin Invest., 2002, 109:69-78). FGF-R4 is mainly expressed in tissues derived from the endoderm, such as the gastrointestinal tract, the pancreas, the liver, the muscles and the adrenal glands.

FGF-R4 is known in the literature as having several cellular roles, the principal three of which are described below:

Firstly, this receptor is involved in the control of various cell differentiation processes in vitro and in vivo, such as skeletal muscle differentiation and regeneration, mesenchymal tissue differentiation, or osteogenesis, or else in the formation of alveoli during post-natal hepatic development.

Secondly, FGF-R4 is described in the control of bile acid and cholesterol homeostasis and is thought to be involved in the control of adiposity. Furthermore, the balance between bile acid production and cholesterol production is controlled by FGF-R4 in vitro and in vivo.

Thirdly, FGF-R4 is involved in certain tumoral phenomena such as the development of hepatocellular carcinomas or colon cancers, or in the proliferation of mammary fibroadenoma cells or of mammary cancer epithelial cells, such as mammary or colorectal carcinoma cell motility. The tumoral involvement of FGF-R4 is predominantly associated with the appearance of the polymorphism (Gly388Arg) correlated with the acceleration of tumour progression in mammary and colorectal tumours (Bange and al., Cancer Res. 62/3, 840-847.2002), prostate tumours (Wang and al., Clin. Cancer Res. 10/18, 6169-6178, 2004) or hepatic tumours (Nicholes and al., Am. J. Pathol. 160/6, 2295-307, 2002). This polymorphism is also associated with a poor prognosis in the case of sarcomas (Morimoto and al., Cancer 98/10, 2245-2250, 2003), of pulmonary adenocarcinomas (Spinola and al., J Clin Oncol 23, 7307-7311, 2005) or of squamous sarcomas (da Costa Andrade and al., Exp Mol Pathol 82, 53-7, 2007). The overexpression of FGF-R4 is also described in certain pancreatic cancer lines (Shah and al., Oncogene 21/54, 8251-61, 2002) and correlates with astrocytoma malignancy (Yamada and al., Neurol. Res. 24/3, 244-8, 2002). In addition, the use of an anti-FGF19 monoclonal antibody in in vivo models of colon tumour xenografts or in hepatocellular carcinoma models shows that the inactivation of FGF19 and therefore the blocking of FGF-R4 activation can be beneficial in the treatment of colon or liver cancer.

II is accepted in the literature that the FGF/FGF-R1 and FGF/FGF-R2 pairs participate in the formation of new blood vessels in a normal or pathological context. However, the potential involvement of FGF-R4 in the control of this cell phenomenon has never been studied. It has, in fact, up until now been supposed that the activation of angiogenesis is mediated by FGF-R1 and/or FGF-R2 (Presta and al, Cytokine Growth Factor Rev., 2005, 16:159-78).

Examples of FGF-R4 antagonists are described in the literature, in particular: small molecules, but they do not target FGF-R4 specifically, thus leading to adverse effects. Thus, tyrosine-kinase domain-inhibiting small chemical molecules which inhibit several FGF-Rs and also other receptor tyrosine kinases have been described by Thompson and al. (Thompson and al J Med Chem., 2000, 43:4200-11.). Small chemical molecules which inhibit FGF-Rs by association with their extracellular portion have also been described in application WO2007/080325.

Antibodies have also been studied, such as the anti-FGFR1 and/or anti-FGFR4 antibodies described in international applications WO2005/066211 and WO2008052796 or by Chen and al. (Hybridoma 24/3, 152-159, 2005). Application WO2005/037235 describes antibodies which are FGF-R antagonists, for the treatment of obesity and diabetes. In addition, anti-FGF-R4 antibodies which are agonists are described in application WO03/063893.

The subject of the present invention is an FGF-R4 receptor antagonist, characterized in that it binds specifically to said FGF-R4 receptor.

Advantageously, said antagonist is an antibody specific for the FGF-R4 receptor.

In one embodiment, the antagonist which is the subject of the invention binds to the D2-D3 domain of the FGF-R4 receptor. In one advantageous embodiment, the antagonist which is the subject of the invention binds to the D2 domain of the FGF-R4 receptor. In an even more advantageous embodiment, the antagonist binds to the sequence SEQ ID No. 70.

Advantageously, the FGF-R4 receptor-specific antagonist has a K_{D} with respect to the FGF-R4 receptor, determined by the Biacore technique, of less than 10⁻⁸ M, less than 5×10⁻⁹ M, less than 2×10⁻⁹ M or less than 1×10⁻⁹ M.

In one advantageous embodiment, the FGF-R4 receptor-specific antagonist is active against both human FGF-R4 and murine FGF-R4.

In another advantageous embodiment, the FGF-R4 receptor-specific antagonist is active at the same time against human FGF-R4, murine FGF-R4 and rat FGF-R4.

In one advantageous embodiment, the FGF-R4 receptor-specific antagonist antibody comprises at least one CDR having a sequence identical to SEQ ID No. 9, 10, 11, 12, 13, 14, 73, 74, 75, 78, 79, 80, 83, 84, 85, 88, 89, 90, 93, 94, 95, 98, 99, 100, 103, 104, 105, 108, 109 or 110 or at least one CDR of which the sequence differes by one or two amino acids compared with the sequences SEQ ID No. 9, 10, 11, 12, 13, 14, 73, 74, 75, 78, 79, 80, 83, 84, 85, 88, 89, 90, 93, 94, 95, 98, 99, 100, 103, 104, 105, 108, 109 or 110, provided that the antibody keeps its binding specificity.

In one particularly advantageous embodiment, the FGF-R4 receptor-specific antagonist antibody comprises the CDRs of sequence SEQ ID No. 9, 10, 11, 12, 13, 14, 73, 74, 75, 78, 79, 80, 83, 84, 85, 88, 89, 90, 93, 94, 95, 98, 99, 100, 103, 104, 105, 108, 109 or 110 or CDRs of which the sequences differ by one or two amino acids compared, respectively, with the abovementioned sequences, provided that this does not modify the FGF-R4 receptor-binding specificity of the antibody.

In one advantageous embodiment, the antibodies of the invention comprise at least one heavy chain and at least one light chain, said heavy chain comprising three CDR sequences having amino acid sequences selected from the group constituted of SEQ ID No. 9, 10 and 11 or 73, 74 and 75, 83, 84 and 85, or 93, 94 and 95, or 103, 104 and 105, said light chain comprising three CDR sequences having amino acid sequences selected from the group constituted of SEQ ID No. 12, 13 and 14 or 78, 79 and 80, or 88, 89 and 90, or 98, 99 and 100, or 108, 109 and 110.

In one advantageous embodiment, the heavy chain variable regions of the FGF-R4 receptor-specific antagonist antibody comprise a sequence having at least 80%, 90%, 95% or 99% identity with the sequence SEQ ID No. 6, 77, 87, 97 or 107.

In one advantageous embodiment, the light chain variable regions of the FGF-R4 receptor-specific antagonist antibody comprise a sequence having at least 80%, 90%, 95% or 99% identity with the sequence SEQ ID No. 8, 72, 82, 92 or 102.

A subject of the present invention is also an FGF-R4 receptor-specific antagonist antibody comprising a heavy chain comprising a variable region encoded by a nucleotide sequence having at least 80%, 90%, 95% or 99% identity with sequence SEQ ID No. 5, 76, 86, 96 or 106.

A subject of the present invention is also an FGF-R4 receptor-specific antagonist antibody comprising a light chain comprising a variable region encoded by a nucleotide sequence having at least 80%, 90%, 95% or 99% identity with the sequence SEQ ID No. 7, 71, 81, 91 or 101.

A subject of the present invention is also an FGF-R4 receptor-specific antagonist antibody comprising a heavy chain comprising a variable region of polypeptide sequence SEQ ID No. 6, 77, 87, 97 or 107.

A subject of the present invention is also an FGF-R4 receptor-specific antagonist antibody comprising a light chain comprising a variable region of polypeptide sequence SEQ ID No. 8, 72, 82, 92 or 102.

A subject of the present invention is also an FGF-R4 receptor-specific antagonist antibody comprising sequences encoded by the nucleotide sequences SEQ ID Nos. 5 and 7 or 71 and 76, or 81 and 86, or 91 and 96, or 101 and 106.

A subject of the present invention is also an FGF-R4 receptor-specific antagonist antibody comprising the polypeptide sequences SEQ ID Nos. 6 and 8, or 72 and 77, or 82 and 87, or 92 and 97 or 102 and 107.

In one advantageous embodiment, the FGF-R4 receptor-specific antagonist antibody comprises sequences at least 80%, 90%, 95% or 99% identical to SEQ ID No. 2 and/or SEQ ID No. 4; or SEQ ID No. 72 and/or SEQ ID No. 77; or SEQ ID No. 82 and/or SEQ ID No. 87; or SEQ ID No. 92 and/or SEQ ID No. 97; or SEQ ID No. 102 and/or SEQ ID No. 107.

In one particularly advantageous embodiment, the FGF-R4 receptor-specific antagonist antibody comprises a heavy chain encoded by a nucleotide sequence at least 80%, 90%, 95% or 99% identical to the sequence SEQ ID No. 1.

A subject of the present invention is also an FGF-R4 receptor antagonist antibody comprising a heavy chain of polypeptide sequence at least 80%, 90%, 95% or 99% identical to the sequence SEQ ID No. 2.

A subject of the present invention is also an FGF-R4 receptor-specific antagonist antibody comprising a light chain encoded by a nucleotide sequence at least 80%, 90%, 95% or 99% identical to the sequence SEQ ID No. 3.

A subject of the present invention is also an FGF-R4 receptor-specific antagonist antibody comprising a light chain of polypeptide sequence at least 80%, 90%, 95% or 99% identical to the sequence SEQ ID No. 4.

A subject of the present invention is also an FGF-R4 receptor-specific antagonist antibody comprising the sequences encoded by the nucleotide sequences SEQ ID No. 1 and 3.

In one even more advantageous embodiment, the FGF-R4-specific antagonist antibody comprises a heavy chain comprising the sequence SEQ ID No. 2 and a light chain comprising the sequence SEQ ID No. 4.

The antibody composed of a heavy chain of sequence SEQ ID No. 2 and of a light sequence SEQ ID No. 4 will be called 40-12 in the rest of the application.

In one embodiment of the invention, the FGF-R4-specific antibodies are active at the same time against human FGF-R4, murine FGF-R4 and rat FGF-R4.

In one advantageous embodiment, the FGF-R4 receptor-specific antagonist induces inhibition of AKT/p38 cell pathways.

In one advantageous embodiment, the FGF-R4 receptor-specific antagonist induces inhibition of Erk1/2 cell pathways.

In one advantageous embodiment, the FGF-R4 receptor-specific antagonist induces inhibition of FGF-R4-controlled cell signalling pathways.

In another advantageous embodiment, the FGF-R4 receptor-specific antagonist induces inhibition of tumour cell proliferation.

In yet another advantageous embodiment, the FGF-R4 receptor-specific antagonist induces inhibition of angiogenesis.

In another particularly advantageous embodiment, the FGF-R4 receptor-specific antagonist has an affinity for FGF-R4 which is 10 times greater than its affinity for the other FGF receptors.

In one particularly advantageous embodiment, the antibody according to the invention is an FGF-R4 receptor-specific humanized antagonist antibody.

In one embodiment, the FGF-R4 receptor-specific humanized antagonist antibody comprises a light chain of which the variable region is encoded by a nucleotide sequence at least 80%, 90%, 95% or 99% identical to the sequence SEQ ID No. 29 or the sequence SEQ ID No. 31.

In another embodiment, the FGF-R4 receptor-specific humanized antagonist antibody comprises a light chain of which the variable region is at least 80%, 90%, 95% or 99% identical to the sequence SEQ ID No. 30 or to the sequence SEQ ID No. 32.

In another embodiment, the FGF-R4 receptor-specific humanized antagonist antibody comprises a light chain in which the variable region is encoded by a sequence identical to the nucleotide sequence SEQ ID No. 29 or to the sequence SEQ ID No. 31.

A subject of the present invention is also an FGF-R4 receptor-specific humanized antagonist antibody comprising a heavy chain of which the variable region is encoded by a sequence at least 80%, 90%, 95% or 99% identical to the sequence SEQ ID No. 33, to the sequence SEQ ID No. 35 or to the sequence SEQ ID No. 37.

A subject of the present invention is also an FGF-R4 receptor-specific humanized antagonist antibody comprising a heavy chain of which the variable region is at least 80%, 90%, 95% or 99% identical to the sequence SEQ ID No. 34, to the sequence SEQ ID No. 36 or to the sequence SEQ ID No. 38.

A subject of the present invention is also an FGF-R4 receptor-specific humanized antagonist antibody comprising a heavy chain encoded by a nucleotide sequence SEQ ID No. 33 and/or SEQ ID No. 35 and/or SEQ ID No. 37.

A subject of the present invention is also an FGF-R4 receptor-specific humanized antagonist antibody of which the humanized sequences of sequence SEQ ID No. 30 or 32 are used in combination with the humanized sequences of sequence SEQ ID No. 34, 36 or 38.

In another embodiment, the FGF-R4 receptor-specific antagonist antibody comprises the CDRs of sequence SEQ ID Nos. 73, 74, 75, 78, 79 and 80 or CDRs of which the sequences differ by one or two amino acids compared, respectively, with the abovementioned sequences, provided that this does not modify the FGF-R4 receptor-binding specificity of the antibody.

In another embodiment, the FGF-R4 receptor-specific antagonist antibody comprises the CDRs of sequence SEQ ID Nos. 83, 84, 85, 88, 89 and 90 or CDRs of which the sequences differ by one or two amino acids compared, respectively, with the abovementioned sequences, provided that this does not modify the FGF-R4 receptor-binding specificity of the antibody.

In another embodiment, the FGF-R4 receptor-specific antagonist antibody comprises the CDRs of sequence SEQ ID Nos. 93, 94, 95, 98, 99 and 100 or CDRs of which the sequences differ by one or two amino acids compared, respectively, with the abovementioned sequences, provided that this does not modify the FGF-R4 receptor-binding specificity of the antibody.

In another embodiment, the FGF-R4 receptor-specific antagonist antibody comprises the CDRs of sequence SEQ ID Nos. 103, 104, 105, 108, 109 and 110 or CDRs of which the sequences differ by one or two amino acids compared, respectively, with the abovementioned sequences, provided that this does not modify the FGF-R4 receptor-binding specificity of the antibody.

In one preferred embodiment of the invention, the FGF-R4 receptor-specific antagonist antibody comprises the CDRs of sequence SEQ ID Nos. 83, 84, 85, 88, 89 and 90.

In one advantageous embodiment, the FGF-R4 receptor-specific antagonist antibody is a human antibody of which the heavy chain variable regions comprise a nucleotide sequence having at least 80%, 90%, 95% or 99% identity with the sequence SEQ ID No. 76, 86, 96 or 106.

In one advantageous embodiment, the FGF-R4 receptor-specific antagonist antibody is a human antibody of which the light chain variable regions comprise a nucleotide sequence having at least 80%, 90%, 95% or 99% identity with the sequence SEQ ID No. 71, 81, 91 or 101.

A subject of the present invention is also an FGF-R4 receptor-specific human antagonist antibody comprising a heavy chain comprising a variable region encoded by a protein sequence having at least 80%, 90%, 95% or 99% identity with the sequence SEQ ID No. 77, 87, 97 or 107.

A subject of the present invention is also an FGF-R4 receptor-specific human antagonist antibody comprising a light chain comprising a variable region encoded by a protein sequence having at least 80%, 90%, 95% or 99% identity with the sequence SEQ ID No. 72, 82, 92 or 102.

A subject of the present invention is also an FGF-R4 receptor-specific human antagonist antibody comprising sequences encoded by the nucleotide sequences SEQ ID Nos. 71 and 76 or the nucleotide sequences SEQ ID Nos. 81 and 86 or the nucleotide sequences SEQ ID Nos. 91 and 96 or the nucleotide sequences SEQ ID Nos. 101 and 106.

A subject of the present invention is also an FGF-R4 receptor-specific human antagonist antibody comprising the polypeptide sequences SEQ ID Nos. 72 and 77 or the polypeptide sequences SEQ ID Nos. 82 and 87 or the polypeptide sequences SEQ ID Nos. 92 and 97 or the polypeptide sequences SEQ ID Nos 102 and 107.

In one advantageous embodiment, the FGF-R4 receptor-specific antagonist antibody comprises sequences at least 80%, 90%, 95% or 99% identical to SEQ ID No. 72 and/or SEQ ID No. 77.

In another advantageous embodiment, the FGF-R4 receptor-specific human antagonist antibody comprises sequences at least 80%, 90%, 95% or 99% identical to SEQ ID No. 82 and/or SEQ ID No. 87.

In another advantageous embodiment, the FGF-R4 receptor-specific human antagonist antibody comprises sequences at least 80%, 90%, 95% or 99% identical to SEQ ID No. 92 and/or SEQ ID No. 97.

In another advantageous embodiment, the FGF-R4 receptor-specific human antagonist antibody comprises sequences at least 80%, 90%, 95% or 99% identical to SEQ ID No. 102 and/or SEQ ID No. 107.

In one preferred embodiment, a subject of the present invention is an FGF-R4 receptor-specific human antagonist antibody comprising a light chain encoded by a nucleotide sequence at least 80%, 90%, 95% or 99% identical to the sequence SEQ ID No. 82 and comprising a light chain of polypeptide sequence at least 80%, 90%, 95% or 99% identical to the sequence SEQ ID No. 87.

Even more preferably, a subject of the present invention is an FGF-R4 receptor-specific human antagonist antibody comprising sequences encoded by the nucleotide sequences SEQ ID Nos. 82 and 87.

The antibody composed of a heavy chain of sequence SEQ ID No. 77 and of a light sequence SEQ ID No. 72 will be called clone 8 in the rest of the application.

The antibody composed of a heavy chain of sequence SEQ ID No. 87 and of a light sequence SEQ ID No. 82 will be called clone 31 in the rest of the application.

The antibody composed of a heavy chain of sequence SEQ ID No. 97 and of a light sequence SEQ ID No. 92 will be called clone 33 in the rest of the application.

The antibody composed of a heavy chain of sequence SEQ ID No. 107 and of a light sequence SEQ ID No. 102 will be called clone 36 in the rest of the application.

The field of the present invention is not limited to the antibodies comprising these sequences. In fact, all the antibodies that specifically bind to FGF-R4, having an antagonistic action on this receptor, are part of the field of the present invention.

A subject of the present invention is also an FGF-R4 receptor-specific antagonist antibody conjugated to a cytotoxic agent.

A subject of the present invention is the use of an FGF-R4 receptor-specific antagonist in the treatment of diseases associated with angiogenesis.

A subject of the present invention is the use of an FGF-R4 receptor-specific antagonist in the treatment of a cancer.

A subject of the present invention is the use of an FGF-R4 receptor-specific antagonist in the treatment of hepatocarcinomas or of any other type of hepatic cancer.

A subject of the present invention is the use of an FGF-R4 receptor-specific antagonist in the treatment of pancreatic cancer.

A subject of the present invention is an FGF-R4 receptor-specific antibody which is of use both in the treatment of diseases associated with angiogenesis and in the treatment of hepatocarcinomas or of any other type of hepatic cancer.

A subject of the present invention is an FGF-R4 receptor-specific antibody which is of use at the same time in the treatment of diseases associated with angiogenesis, in the treatment of hepatocarcinomas or of any other type of hepatic cancer, and in the treatment of pancreatic cancer, or cancer of the organs of the gastrointestinal tract or any other organ expressing FGF-R4.

A subject of the present invention is a pharmaceutical composition comprising an FGF-R4 receptor-specific antagonist and excipients.

A subject of the present invention is a method of treating a cancer, comprising the administration, to the patient, of an FGF-R4 receptor-specific antagonist antibody.

A subject of the present invention is a method of treating a disease associated with a pathological increase in angiogenesis, comprising the administration, to the patient, of an FGF-R4 receptor-specific antagonist antibody.

A subject of the present invention is a method of selecting an FGF-R4 receptor-specific antagonist monoclonal antibody, comprising the following steps:
a. immunizing mice,
b. taking lymph nodes from the mice, and
c. screening the hybridoma supernatants.

A subject of the present invention is a cell line that produces FGF-R4 receptor-specific antagonist antibodies.

A subject of the present invention is a method of producing an FGF-R4 receptor-specific antagonist antibody, comprising culturing a cell line that produces FGF-R4 receptor antagonist antibodies.

A subject of the present invention is a drug comprising an FGF-R4 receptor-specific antagonist.

A subject of the present invention is also a polynucleotide encoding a polypeptide selected from the group constituted of SEQ ID Nos. 2, 4, 6, 8, 9, 10, 11, 12, 13, 14, 30, 32, 34, 36, 38, 72, 73, 74, 75, 77, 78, 79, 80, 82, 83, 84, 85, 87, 88, 89, 90, 92, 93, 94, 95, 97, 98, 99, 100, 102, 103, 104, 105, 107, 108, 109 and 110, and the sequences at least 80%, 90%, 95% or 99% identical to one of these sequences.

A subject of the present invention is a recombinant vector comprising a polynucleotide as described above or encoding a polypeptide as described above.

In order to enable the expression of heavy chains and/or light chains of the FGF-R4 receptor antagonist antibody, which is a subject of the invention, the polynucleotides encoding said chains are inserted into expression vectors. These expression vectors may be plasmids, YACs, cosmids, retroviruses, EBV-derived episomes, and any of the vectors that those skilled in the art may deem appropriate for the expression of said chains.

A subject of the present invention is a host cell comprising a recombinant vector as described above.

### DETAILED DESCRIPTION OF THE INVENTION

A subject of the present invention is the use of antibodies specifically directed against FGF-R4 (without cross-reaction with FGF-R1, R2 or R3) for inhibiting angiogenesis and tumour growth.

Unexpectedly, the inventors have shown that FGF-R4 plays an active and specific role in the control of angiogenesis.

This function of FGF-R4 had never previously been shown or proposed. Consequently, this receptor may be used as a target for treating pathologies exhibiting an angiogenic dysfunction. The FGF-R4 ligands capable of modulating the activity of said receptor are therefore potential therapeutic agents for numerous angiogenesis-related pathologies.

The present invention can therefore be used in the treatment of all pathologies involving a dysregulation of angiogenesis and requiring inhibition thereof. The pathologies covered may be cancer, with the use of the antagonists according to the invention as tumour angiogenesis inhibitors, or pathologies for which a dysregulation of angiogenesis is described, such as: age-related macular degeneration or ARMD, inflammatory diseases such as rheumatoid arthritis, osteoarthritis, colitis, ulcers or any inflammatory disease of the intestines, atherosclerosis, or else in the treatment of obesity.

The use of these antibodies is also illustrated in the inhibition of tumour growth. The antagonists according to the present invention can therefore be used for the treatment of certain cancers involving a dysregulation of FGF-R4, and more particularly, liver cancer, colon cancer, breast cancer, lung cancer, prostate cancer, pancreatic cancer, skin cancer or oesophageal cancer.

One of the major advantages of the antagonists according to the present invention is to specifically target an FGF receptor, in the case in point FGF-R4. This specificity makes it possible to limit the adverse effects that small chemical molecules which inhibit the tyrosine kinase domain can have. In addition, since FGF-R4 is not expressed ubiquitously, but is in particular expressed on endothelial cells, for instance on hepatocyte cells, biliary cells, mammary cells, prostatic cells, ovarian cells, pancreatic cells or renal cells, this provides a method of treating diseases related to a dysregulation of FGF-R4 activity which limits the side effects.

The term “antagonist” refers to any ligand capable of reducing or completely inhibiting the activity of FGF-R4. This antagonist compound is thus also referred to as FGF-R4 inhibitor.

This antagonist may be any FGF-R4 ligand, such as a chemical molecule, a recombinant protein, an oligosaccharide, a polysaccharide, an oligonucleotide or an antibody capable of specifically binding to the FGF-R4 receptor, with the exclusion of any other FGFR.

A subject of the invention is therefore an FGF-R4-specific antagonist. According to the invention, an antagonist that binds specifically to FGF-R4 refers to a ligand which doe's not bind to the other FGF receptors, namely FGF-R1, FGF-R2 or FGF-R3. In particular, an FGF-R4-specific antibody is an antibody which does not exhibit any cross-reaction with FGF-R1, FGF-R2 or FGF-R3.

“Specific binding” refers to a difference, by a factor of at least 10, between the intensity of the binding to one receptor compared with another, in this case between the binding to FGF-R4 and the possible bindings to FGF-R1, FGF-R2 or FGF-R3.

In one embodiment of the invention, the FGF-R4 ligand is an oligosaccharide or a polysaccharide.

An “oligosaccharide” refers to any saccharide polymer containing from three to ten units of simple sugars. Natural oligosaccharides such as, for example, fructo-oligosaccharides (FOS), and synthetic oligosaccharides such as, for example, heparin-mimetic antithrombotics, exist.

The term “polysaccharide” refers to any polymer constituted of more than ten monosaccharides linked to one another by glycosidic linkages. Natural polysaccharides such as, for example, mucopolysaccharides, fucoids, carrageenans or bacterial exopolysaccharides exist, as do synthetic polysaccharides. Thus, low-molecular-weight fucoidans or highly sulphated exopolysaccharides have shown pro-angiogenic activities (Chabut and al., Mol Pharmacol., 2003, 64:696-702; Matou and al., Biochem Pharmacol., 2005, 69:751-9). Conversely, heparin-derived weakly sulphated oligosaccharides or phosphomannopentose sulphates can have anti-angiogenic characteristics (Parish and al., 1999, 15:3433-41; Casu and al., J Med Chem., 2004, 12:838-48)

In one embodiment of the invention, the FGF-R4 ligand is an antibody.

The term “antibody” refers to antibodies or derived molecules of any type, such as polyclonal and monoclonal antibodies. Included among the molecules derived from monoclonal antibodies are humanized antibodies, human antibodies, multispecific antibodies, chimeric antibodies, antibody fragments, nanobodies, etc.

In one embodiment of the invention, the FGF-R4-specific antagonist is a polyclonal antibody.

A “polyclonal antibody” is an antibody which has been produced from a mixture of antibodies originating from several B lymphocyte clones and which recognize a series of different epitopes.

In one advantageous embodiment, the FGF-R4-specific antagonist is a monoclonal antibody.

A “monoclonal antibody” is an antibody obtained from a substantially homogeneous population of antibodies derived from a single type of B lymphocyte, clonally amplified. The antibodies making up this population are identical except for possible naturally occurring mutations that may be present in minor amounts. These antibodies are directed against a single epitope and are therefore highly specific.

The term “epitope” refers to the site of the antigen to which the antibody binds. If the antigen is a polymer, such as a protein or a polysaccharide, the epitope may be made up of contiguous or noncontiguous residues.

In one advantageous embodiment of the invention, the anti-FGF-R4 antagonist antibody binds to an epitope belonging to the D2-D3 domain of the FGF-R4 receptor.

In an even more advantageous embodiment, the antibody binds to an epitope included in the domain comprising amino acids 144 to 365 of the FGF-R4 receptor.

In an even more advantageous embodiment, the antibody binds to an epitope included in the D2 domain of the FGF-R4 receptor, this epitope corresponding to amino acids 145 to 242 described in the sequence SEQ ID No. 70.

An antibody, also known as an immunoglobulin, is composed of two identical heavy chains (“VH”) and of two identical light chains (“VL”) which are linked by a disulphide bridge. Each chain contains a constant region and a variable region. Each variable region comprises three segments called “complementarity determining regions” (“CDRs”) or “hypervariable regions”, which are mainly responsible for the binding to the epitope of an antigen.

The term “VH” refers to the variable regions of an immunoglobulin heavy chain of an antibody, including the heavy chains of an Fv, scFv, dsFv, Fab, Fab′ or F(ab)′ fragment.

The term “VL” refers to the variable regions of an immunoglobulin light chain of an antibody, including the light chains of an Fv, scFv, dsFv, Fab, Fab′ or F(ab)′ fragment.

The term “antibody fragment” refers to any part of said antibody: Fab (fragment antigen binding), Fv, scFv (single chain Fv), Fc (fragment crystallizable). Preferably, these functional fragments will be fragments of the type Fv, scFv, Fab, F(ab′)2, Fab′, scFv-Fc or diabodies, which generally have the same binding specificity as the chimeric or humanized, monoclonal antibody from which they are derived. According to the present invention, antibody fragments of the invention can be obtained from chimeric or humanized, monoclonal antibodies by methods such as digestion with enzymes, for instance pepsin or papain, and/or by cleavage of the disulphide bridges by chemical reduction.

The term “CDR regions or CDRs” is intended to denote the immunoglobulin heavy and light chain hypervariable regions as defined by Kabat and al. (Kabat and al., Sequences of proteins of immunological interest, 5th Ed., U.S. Department of Health and Human Services, NIH, 1991, and later editions). There are 3 heavy chain CDRs and 3 light chain CDRs. The term CDR or CDRs is used herein to denote, as appropriate, one or more of these regions or even all of these regions which contain the majority of the amino acid residues responsible for the affinity binding of the antibody for the antigen or the epitope that it recognizes. The most conserved regions of the variable domain are called FR (for “framework”) regions or sequences.

In another embodiment of the invention, the FGF-R4-specific antagonist is a chimeric antibody.

The term “chimeric antibody” refers to an antibody in which the constant region, or a portion thereof, is altered, replaced or exchanged, such that the variable region is linked to a constant region of a different species, or belongs to another antibody class or subclass.

The term “chimeric antibody” also refers to an antibody in which the variable region, or a portion thereof, is altered, replaced or exchanged, such that the constant region is linked to a variable region of a different species, or belongs to another antibody class or subclass.

The methods of producing chimeric antibodies are known to those skilled in the art. See, for example, Morrison, 1985, Science, 229:1202; Oi and al., 1986, Bio Techniques, 4:214; Gillies and al., 1989, J. Immunol. Methods, 125:191-202; U.S. Pat. Nos. 5,807,715; 4,816,567; and 4,816,397.

These chimeric versions of the antibody may comprise the fusion of the VL and VH variable regions to the Ckappa and the CH (IgG1) constant domains of human origin in order to generate a chimeric monoclonal antibody.

The CH (IgG1) domain can also be modified by point mutations in order to increase the affinity of the Fc fragment for the FcγRIIIa receptor and thereby to increase the effector functions of the antibody (Lazar and al., 2006, Proc. Natl. Acad. Sci. USA 103: 4005-4010; Stavenhagen and al., 2007, Cancer Res. 67: 8882-8890).

The present invention includes the humanized versions of the antibodies.

The term “humanized antibody” refers to an antibody which contains mainly human immunoglobulin sequences. This term generally refers to a non-human immunoglobulin which has been modified by incorporation of human sequences or of residues found in human sequences.

In general, humanized antibodies comprise one or typically two variable domains in which all or part of the CDR regions correspond to parts derived from the non-human parent sequence and in which all or part of the FR regions are those derived from a human immunoglobulin sequence. The humanized antibody can then comprise at least one portion of an immunoglobulin constant region (Fc), in particular that of the human immunoglobulin template chosen.

The goal is thus to have an antibody that is minimally immunogenic in a human. Thus it is possible that one or two amino acids in one or more CDRs can be modified by one that is less immunogenic to a human host, without substantially reducing the specific binding function of the antibody to FGF-R4. Similarly, the residues of the framework regions may not be human, and it is possible for them not to be modified since they do not contribute to the immunogenic potential of the antibody.

Several methods of humanization known to those skilled in the art exist for modifying a non-human parent antibody so as to give an antibody that is less immunogenic to humans. An overall sequence identity with a human antibody is not necessarily required. This is because the overall sequence identity is not necessarily a predictive indicator of reduced immunogenicity, and the modification of a limited number of residues can result in humanized antibodies having a greatly reduced immunogenic potential in humans (Molecular Immunology (2007) 44, 1986-1998).

Various methods are, for example, CDR grafting (EPO 0 239 400; WO 91/09967; and U.S. Pat. Nos. 5,530,101 and 5,585,089), resurfacing (EPO 0 592 106; EPO 0 519 596; Padlan, 1991, Molec Imm 28(415):489-498; Studnicka and al., 1994, Prot Eng 7(6):805-814; and Roguska and al., 1994, PNAS 91:969-973) or else chain shuffling (U.S. Pat. No. 5,565,332).

The present invention relates in particular to humanized antibodies of which the variable portions are modified according to the following technology:

The light and heavy chains most similar to the corresponding chains of the anti-FGF-R4 murine antibody 40-12 are identified by comparison with the Protein Data Bank (H. M. Berman, J. Westbrook, Z. Feng, G. Gilliland, T. N. Bhat, H. Weissig, I. N. Shindyalov, P. E. Bourne. Nucleic Acids Research, 2000, 28:235-242). The sequence alignment uses the BLAST algorithm (J Mol Biol. 1990 October 215:403-410). These are tridimensional structures corresponding to the PDB codes 1NDM & 1ETZ respectively used to build up the homology models of the variable domain light and heavy chains. These tridimensional models are subsequently energy minimized using the standard procedure implemented in the MOE software (Molecular Operating Environment, Chemical Computing Group, Quebec, Canada). A molecular dynamic (MD) simulation of these minimized tridimensional models of the antibody is subsequently performed with the Amber software (D. A. Case, T. E. Cheatham, III, T. Darden, H. Gohlke, R. Luo, K. M. Merz, Jr., A. Onufriev, C. Simmerling, B. Wang and R. Woods. J. Computat. Chem. 2005, 26:1668-1688). This simulation is done with harmonic constraints applied to the protein backbone atoms at a temperature of 500 K for a period of 1.1 nanoseconds in a generalized Born implicit solvent (Gallicchio & Levy, J Comput Chem 2004, 25:479-499). Ten diverse conformations are thus extracted from this first simulation, one tridimensional conformation every one hundred picoseconds, during the last nanosecond of the simulation. These ten diverse conformations are then each subjected to a molecular dynamic simulation, without constraints on the protein backbone, at a temperature of 27° C. for 2.3 nanoseconds in a generalized Born implicit solvent. The bonds involving a hydrogen atom are constrained using the SHAKE algorithm (Barth. and al., J Comp Chem, 1995, 16:1192-1209), the time step is 1 femtosecond, and the simulation was run based on the Langevin equation at constant volume and a constant temperature of 27° C. For each of the ten molecular dynamic simulations, the last two thousand conformations, extracted at a frequency of one every picosecond, are then used to quantify, for each amino acid of the antibody to be humanized, the deviation of the atomic positions with respect to an average, or medoid, conformation of the amino acid. The Scientific Vector Language (SVL) of the MOE software is used to code all of the analysis described below. The medoid conformation of the amino acid is the conformation derived from the molecular dynamic which is the closest to the average conformation calculated from the position of the atoms of all the conformations of the amino acid. The distance used for detecting the medoid conformation is the route mean square (RMSD) of the scalar distances between the atoms of two conformations of the amino acid. Similarly, the deviation of the positions of the atoms of one conformation of an amino acid compared with the medoid conformation is quantified by calculating the RMSD of the scalar distances between the atoms of the amino acid of one conformation of the simulation and the same atoms of the medoid conformation. Subsequently, by comparing the RMSD of the positions of the atoms of a given amino acid (i), averaged over all the ten molecular dynamic simulations (Fi), with the RMSD of the positions of all the amino acids of the antibody, averaged over all the ten molecular dynamic simulations (Fm), it is decided whether the amino acid is flexible enough to be considered able to potentially interact with the T-cell receptors and trigger activation of the immune system. An amino acid i is considered flexible if its flexibility score Zi, defined as Zi=(Fi−Fm)/Fm, is above 0.15. 45 amino acids are thus identified as flexible in the variable domain of the antibody, with the exclusion of the antigen complementarity determining region (CDR) and of its immediate vicinity. The immediate vicinity of the CDR is defined as any amino acid with an alpha carbon at a distance of 5 angstroms (Å) or less to an alpha carbon of the CDR.

The motions of the 60 most flexible amino acids of the antibody, during the nanoseconds (10×2 ns) of simulation, are then compared to the motions of the corresponding amino acids of 49 homology models of human antibody germ lines, for each of which ten molecular dynamic simulations (10×2 ns) have been run using the same protocol. The 60 most flexible amino acids exclude the antigen complementarity determining region (CDR) and its immediate vicinity. The 49 human antibody germ line models were built by systematically combining the 7 most frequent human light chains (vk1, vk2, vk3, vk4, vlambda1, vlambda2, vlambda3) and the 7 most frequent human heavy chains (vh1a, vh1b, vh2, vh3, vh4, vh5, vh6) (Nucleic Acids Research, 2005, Vol. 33, Database issue D593-D597).

The similarity of the antibody to be humanized to the 49 human germ line models is quantified by sampling the positions of specific atoms of the 60 flexible amino acids of an antibody, over the course of the ten molecular dynamic simulations, by means of a unique tridimensional cubic grid which has a 1 Å resolution. This is referred to as quadridimensional similarity. The tridimensional grid used is made of 445 740 points and is initialized using the tridimensional structure of the human antibody corresponding to the PDB code 8FAB. The 8FAB structure is also used to position all the conformations of an antibody to be sampled in the tridimensional grid. For this purpose, the medoid conformation of the molecular dynamic of the antibody is superposed onto the 8FAB structure. This superposition consists of aligning the moments of inertia of the two conformations, followed by the optimization of the scalar distances between the alpha carbon atoms of both conformations. All the remaining conformations of the molecular dynamic of the antibody are superposed onto the medoid conformation using the same method.

Two types of sampling are performed, which result in two similarities (electrostatic similarity and lipophilic similarity), for a pair of antibodies being compared. These two similarities are then added to obtain the total similarity. The first sampling, the electrostatic sampling, considers all atoms of the amino acid side chain. The value in one point, x, of the grid is obtained by applying, to the atoms of the amino acid side chain, a tridimensional Gaussian function f(x) weighted with the atomic partial charge as described in the Amber99 force field (Cieplak, J., and al.; J. Comp. Chem. 2001, 22:1048-1057). The f(x) function is applied on the 3 Cartesian coordinate axes and corresponds to the following formula:

$$f ( x ) = {( s \sqrt{2 π} )}^{- 3} \times exp ( \frac{- {( x - u )}^{2}}{2 {s}^{2}} ) ,$$

with x and u being, respectively, the Cartesian coordinates of a grid point x and of a sampled atom, and s=r/1.6 (r=covalence radius of the atom). The sampling is repeated for all conformations of the amino acid and the obtained results are averaged at all points x of the tridimensional grid. The second sampling, the lipophilic sampling, considers only the lipophilic atoms of the amino acid side chain. The value at one point, x, of the grid is calculated with the same Gaussian function f(x) without weighting. As a result, the two ensembles of conformations from the molecular dynamic simulations, of the two antibodies being compared, are sampled by the same tridimensional grid. The electrostatic similarity (sim-elec) between antibody a and antibody b is measured with the following formula:

$$sim - elec = \frac{{\sum }_{i = 1}^{445740} ( \vert {x}_{i}^{a} + {x}_{i}^{b} \vert - \vert {x}_{i}^{a} - {x}_{i}^{b} \vert )}{{\sum }_{i = 1}^{445740} ( \vert {x}_{i}^{a} + {x}_{i}^{b} \vert )} .$$

The lipophilic similarity is calculated with the same formula applied to the data generated by the lipophilic sampling previously described.

The human germ line model vlambda2-vh2 thus displays the highest quadridimensional similarity (total similarity=58%) of these 60 flexible amino acids with respect to the flexible amino acids of the murine antibody 40-12. The human germ line model vlambda2-vh2 has thus been used to humanize the antibody to be humanized, while focusing on the 45 flexible amino acids. In order to determine the mutations to be made, the tridimensional structure of the model of the murine antibody 40-12 is superposed on that of the model derived from the germ lines showing the highest similarity, with the positions of the alpha carbons of the amino acids being optimized. The amino acids identified as flexible are mutated with the corresponding amino acids in the sequence of the model showing the highest similarity.

The unwanted sequence motifs considered are the following: Aspartate-Proline (peptide bond labile in acidic medium), Asparagine-X-Serine/Threonine (glycosylation site, X=any amino acid except Proline), Aspartate-Glycine/Serine/Threonine (potential formation of succinimide/isoaspartate in the flexible zones), Asparagine-Glycine/Histidine/Serine/Alanine/Cysteine (exposed deamidation sites), Methionine (oxidation in the exposed zones). The humanized sequences thus obtained are finally compared, by means of the BLAST sequence comparison algorithm, with the sequences of the IEDB database (http://www.immuneepitope.org/ The immune epitope database and analysis resource: from vision to blueprint. PLoS Biol. 2005 March; 3(3):e91) so as to be sure that the sequences do not contain any epitopes known to be recognized by B and T lympocytes. If the sequence contains residues which have unwanted sequences, they are then also modified. If the composite sequence contains a known epitope listed in the IEDB, another germ line structure template showing a high similarity is then used as model.

More advantageously, the antibody according to the invention comprises sequences having at least 80%, 90%, 95% or 99% identity with the sequence SEQ ID No. 30 or the sequence SEQ ID No. 32 are used, in combination with the sequences having at least 80%, 90%, 95% or 99% identity with the sequence SEQ ID No. 34, the sequence SEQ ID No. 36 or the sequence SEQ ID No. 38.

In one embodiment, the antibody according to the invention comprises variable light chains of sequence SEQ ID No. 30 and variable heavy chains of sequence SEQ ID No. 34.

In another embodiment, the antibody comprises variable light chains of sequence SEQ ID No. 32 and variable heavy chains of sequence SEQ ID No. 38.

In another embodiment, the antibody comprises variable light chains of sequence SEQ ID No. 30 and variable heavy chains of sequence SEQ ID No. 36.

In another embodiment, the antibody comprises variable light chains of sequence SEQ ID No. 32 and variable heavy chains of sequence SEQ ID No. 34.

In another embodiment, the antibody comprises variable light chains of sequence SEQ ID No. 32 and variable heavy chains of sequence SEQ ID No. 36.

In another embodiment, the antibody comprises variable light chains of sequence SEQ ID No. 30 and variable heavy chains of sequence SEQ ID No. 38.

A subject of the present invention is also FGF-R4-specific human antagonist antibodies. Such antibodies can be obtained by phage display according to methods known to those skilled in the art (McCafferty J. and al, 1990; Hoogenboom, H R and al, 2005). Other technologies are available for the preparation of human antibodies, such as the XenoMouse technology described in U.S. Pat. No. 5,939,598.

In one particular embodiment, the FGF-R4 receptor-specific antagonist antibody is a human antibody of which the heavy chain variable regions comprise a sequence having at least 80%, 90%, 95% or 99% identity with the sequence SEQ ID No. 76, 86, 96 or 106.

In another embodiment, the FGF-R4 receptor-specific antagonist antibody is a human antibody of which the light chain variable regions comprise a sequence having at least 80%, 90%, 95% or 99% identity with the sequence SEQ ID No. 71, 81, 91 or 101.

A subject of the present invention is also an FGF-R4 receptor-specific human antagonist antibody comprising a heavy chain comprising a variable region encoded by a nucleotide sequence having at least 80%, 90%, 95% or 99% identity with the sequence SEQ ID No. 77, 87, 97 or 107.

A subject of the present invention is also an FGF-R4 receptor-specific human antagonist antibody comprising a light chain comprising a variable region encoded by a nucleotide sequence having at least 80%, 90%, 95% or 99% identity with the sequence SEQ ID No. 72, 82, 92 or 102.

A subject of the present invention is also an FGF-R4 receptor-specific human antagonist antibody comprising sequences encoded by the nucleotide sequences SEQ ID Nos. 71 and 76 or the nucleotide sequences SEQ ID Nos. 81 and 86 or the nucleotide sequences SEQ ID Nos. 91 and 96 or the nucleotide sequences SEQ ID Nos. 101 and 106.

A subject of the present invention is also an FGF-R4 receptor-specific human antagonist antibody comprising the polypeptide sequences SEQ ID Nos. 72 and 77 or the polypeptide sequences SEQ ID Nos. 82 and 87 or the polypeptide sequences SEQ ID Nos. 92 and 97 or the polypeptide sequences SEQ ID Nos. 102 and 107.

Particularly preferably, an FGF-R4 receptor-specific human antagonist antibody comprises the polypeptide sequences SEQ ID Nos. 82 and 87.

The amino acid sequences thus modified can also be modified by means of post-translational modifications during the production in the mammalian cell. In particular, the use of stable lines deficient in fucose biosynthesis can make it possible to produce monoclonal antibodies in which the N-glycan of the Fc (position N297) partially or completely lacks fucose and makes it possible to increase the ADCC effector effect (Kanda and al., 2006, Biotechnol. Bioeng. 94:680-688 and Ripka and al., 1986 Arch Biochem Bioph 249: 533-545).

In another embodiment of the invention, the FGF-R4-specific antagonist is a conjugated antibody.

The antibodies may be conjugated to a cytotoxic agent. The term “cytotoxic agent” denotes herein a substance which reduces or blocks the function or the growth of the cells, or causes destruction of the cells.

In one embodiment, the antibody or a binding fragment thereof can be conjugated to a drug, such as a maytansinoid, so as to form a “prodrug” which has cytotoxicity with respect to the cells expressing the antigen.

The cytotoxic agent of the present invention may be any compound which results in the death of a cell, or induces the death of a cell, or decreases cell viability in various ways. The preferred cytotoxic agents include, for example, maytansinoids and maytansinoid analogues, taxoids, CC-1065 and CC-1065 analogues, dolastatin and dolastatin analogues, defined above. These cytotoxic agents are conjugated to antibodies, antibody fragments, functional equivalents, improved antibodies and analogues thereof, as described in the present application.

The conjugated antibodies may be prepared by in vitro methods. A linker group is used to link a drug or a prodrug to the antibody. Suitable linker groups are well known to those skilled in the art and include, in particular, disulphide groups, thioether groups, labile acid groups, photolabile groups, labile peptidase groups and labile esterase groups. Preferred linker groups are disulphide groups and thioether groups. For example, a conjugate can be constructed by using a disulphide exchange reaction or by forming a thioether bridge between the antibody and the drug or prodrug.

Compounds such as: methotrexate, daunorubicin, vincristine, vinblastine, melphalan, mitomycin C, chlorambucil, calicheamicin, tubulysin and tubulysin analogues, duocarmycin and duocarmycin analogues, dolastatin and dolastatin analogues, are also suitable for the preparation of conjugates of the present invention. The molecules may also be linked to the antibody molecules via an intermediate molecule such as serum albumin. Doxorubicin and doxorubicin compounds, as described, for example, in patent application U.S. Ser. No. 09/740,991, may also be useful cytotoxic agents.

The antibodies which are the subject of the present invention may be combined with a cytotoxic molecule or compound. They may also be combined with an anti-angiogenic compound that acts on other angiogenic pathways.

The expression “cells expressing FGF-R4” refers to any eukaryotic cell, especially mammalian cell, and in particular human cell, which expresses an FGF-R4 receptor in its native form or in a mutated form. The FGF-R4 may also be in its whole form or in a truncated form comprising, for example, the extracellular domain of FGR-R4, and in particular the D2-D3 domains. The FGF-R4 may also be recombined in a chimeric form.

The compounds of the invention may be formulated in pharmaceutical compositions for the purpose of topical, oral, parenteral, intranasal, intravenous, intramuscular, subcutaneous, intraocular, etc., administration. Preferably, the pharmaceutical compositions contain carriers that are pharmaceutically acceptable for an injectable formulation. They may in particular be sterile, isotonic, saline solutions (monosodium phosphate, disodium phosphate, sodium chloride, potassium chloride, calcium chloride or magnesium chloride etc., or mixtures of such salts), or dry, in particular lyophilized, compositions which by means of the addition, as appropriate, of sterilized water or physiological saline, can form injectable solutes.

The pathologies targeted may be all diseases related to angiogenesis, whether said angiogenesis is tumoral or non-tumoral.

The pathologies targeted may be cancer (with the use of the invention as an inhibitor of tumour angiogenesis), in particular liver cancer, colon cancer, breast cancer, lung cancer, prostate cancer, pancreatic cancer or skin cancer, or else pathologies for which a dysregulation of angiogenesis is described, such as: age-related macular degeneration or ARMD, inflammatory diseases such as rheumatoid arthritis, osteoarthritis, colitis, ulcers or any inflammatory disease of the intestines, atherosclerosis or else in the treatment of obesity.

The anti-FGF-R4 antibodies may also be used for treating cancer, in particular hepatocarcinomas and other hepatic cancers, and pancreatic cancer as an inhibitor having an action directly on tumour growth.

The present invention is illustrated, without however being limited, by the examples which follow.

### DESCRIPTION OF THE FIGURES

FIGS. 1A and 1B: In vitro (FIG. 1A), human endothelial cells of HUVEC type are capable of forming a network of pseudotubules, known as angiogenesis. This network is stimulated by the addition of 1 ng/ml of FGF2. This induction can also be obtained by adding 10 ng/ml of FGF19, which is a ligand specific for FGF-R4, whereas 10 ng/ml of FGF4 (ligand which does not activate FGF-R4) are not capable of stimulating angiogenesis. In the same way, in vivo (FIG. 1B) in a murine model of induction of angiogenesis in a sponge implant on the back of the mouse, FGF2 is capable of inducing the recruitment of functional neovessels to the sponge, which is characterized by an increase in the haemoglobin content of these sponges in comparison with the control. FGF19 is also capable of inducing this angiogenesis in the sponge.

FIG. 2A: Map of the plasmid pXL4614 enabling the expression of hFGFR4-Histag (SEQ ID No. 40).

FIG. 2B: Map of the plasmid pXL4613 enabling the expression of hFGFR4-Streptag (SEQ ID No. 69).

FIG. 3: Map of the plasmid pXL4615 enabling the expression of hFGFR4(D2,D3)-Histag (SEQ ID No. 42).

FIG. 4: Map of the plasmid pXL4621 enabling the expression of mFGFR4-Histag (SEQ ID No. 44).

FIG. 5: Map of the plasmid pXL4328 enabling the expression of hFGFR1-Fc (of sequence SEQ ID No. 46).

FIG. 6: Map of the plasmid pXL4327 enabling the expression of hFGFR2-Fc (of sequence SEQ ID No. 48).

FIG. 7: ELISA plates are coated with the 4 human FGF receptors. The ability of the anti-FGFR4 antibodies 40-12 and 64-12 to recognize these various FGF-Rs is measured by ELISA assay. Clone 40-12 (black histogram) is specific for FGF-R4. Clone 64-12 (grey histogram) recognizes, in addition, FGF-R3 very weakly.

FIGS. 8A and 8B: The active antagonist antibody 40-12 and its inactive control 64-12 have no effect per se on basal angiogenesis. On the other hand, at the dose of 30 μg/ml (approximately 200 nM), the anti-FGFR4 monoclonal antibody 40-12 is capable of inhibiting the FGF2-induced angiogenesis of HUVEC cells, whereas the control antibody 64-12 is not capable of doing so (FIG. 8A).

FGF2 labelled with an AlexaFluor® 488 nm is capable of binding to the FGF-R4 expressed by 300-19 cells (white histogram). This interaction can be dissociated by unlabelled FGF2 (black histograms) or by the anti-FGFR4 antibody 40-12 (dark grey histograms), whereas the control antibody 64-12 is not capable of doing so (light grey histograms) (FIG. 8B).

FIGS. 8C and 8D: Effect of the anti-FGFR4 antibodies derived from clones 8, 31, 33 and 36, at 10 μg/ml, on the in vitro angiogenesis induced by 3 ng/ml of FGF-2. Clones 8 (FIG. 8C) and 31, 33 and 36 (FIG. 8D) inhibit the FGF-2-induced angiogenesis of HUVEC cells.

FIGS. 9A and 9B: Hep3b human hepatocarcinoma cells are stimulated with FGF19 at 30 ng/ml. This stimulation induces FGF-R4-specific cell signalling, resulting in synthesis of the cFos and JunB proteins and in the phosphorylation of Erk1/2, observed by Western blotting (FIG. 9A). Each band is then quantified. This quantification is represented in the form of a histogram (FIG. 9B). The antibody 40-12 at 100 μg/ml completely inhibits the induction of the FGF-R4-specific cell signalling, whereas the control antibody has no effect. Specifically, the antibody 40-12 completely blocks the synthesis of JunB and of cFos and also the phosphorylation of Erk1/2 induced by FGF19 after a stimulation of 3 h.

FIG. 9C: The inhibitory effect of the anti-FGFR4 antibody 40-12 on the phosphorylation of Erk1/2 induced in the Hep3b cells by FGF-19 (30 ng/ml) is confirmed by means of the anti-phosphoERk1/2 ELISA. The antibody 40-12 is also capable of inhibiting the phosphorylation of Erk1/2 in Hep3b cells stimulated with FGF-2 (1 ng/ml) or with serum (FCS) at 10%.

FIG. 9D: Percentage inhibition of Erk1/2 phosphorylation induced by FGF-19 (30 ng/ml) obtained by ELISA on Hep3b cells using the antibodies derived from clones 8, 31, 33 and 36.

FIGS. 10A and 10B: The proliferation of Hep3b cells can be stimulated by adding serum (FIG. 10A) or FGF19 (FIG. 10B). The inductin with serum is partially inhibited by the antibody 40-12 at 100 μg/ml, whereas the control antibody has no effect (FIG. 10A). The proliferation induced by FGF19 is completely blocked with 10 μg/ml of anti-FGFR4 antibody 40-12, whereas at the same dose, the control antibody is not capable of doing so (FIG. 10B).

FIGS. 11A to 11D: The anti-FGFR4 antibody 40-12 is capable of reducing the development of pancreatic tumors in a RipTag murine model by inhibiting tumour angiogenesis: The Rip1-Tag2 model is a murine model in which transgenic mice expressing the SV40 T antigen in the insulin-producing 13 cells of the pancreatic islets (Hanahan D. Nature, 1985, 9-15:115-22.). This T antigen is expressed during embryonic development of the pancreas up to 4 to 5 weeks of life, without apparent effect. Some of the pancreatic islets expressing the T antigen then progress, during the next 5 weeks, towards the formation of angiogenic islets associated with activation of the vasculature and then towards the development of small tumours of adenoma type. A few weeks later, some adenomas develop, so as to form invasive carcinomas (FIG. 11A).

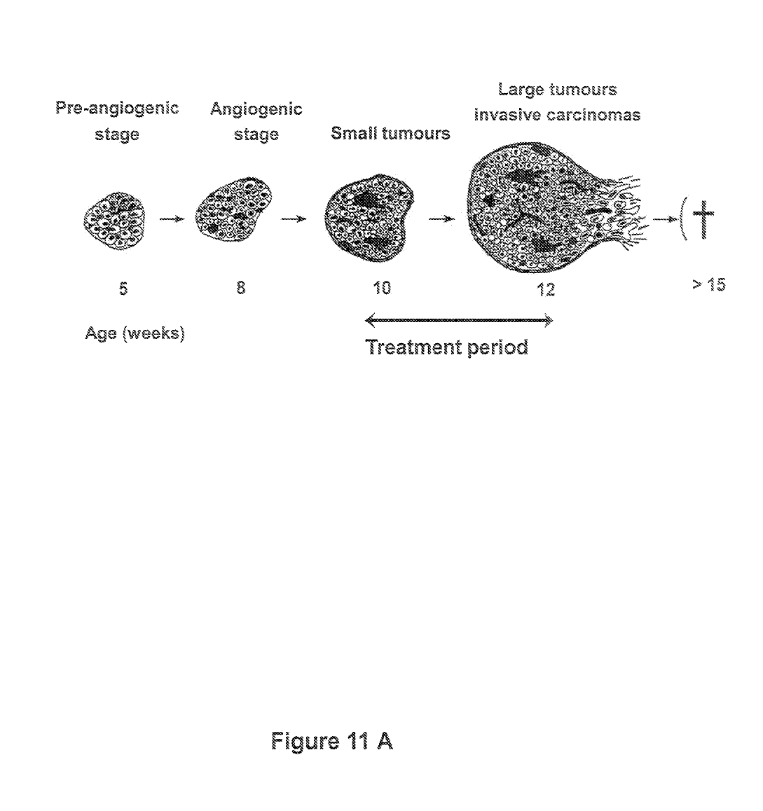

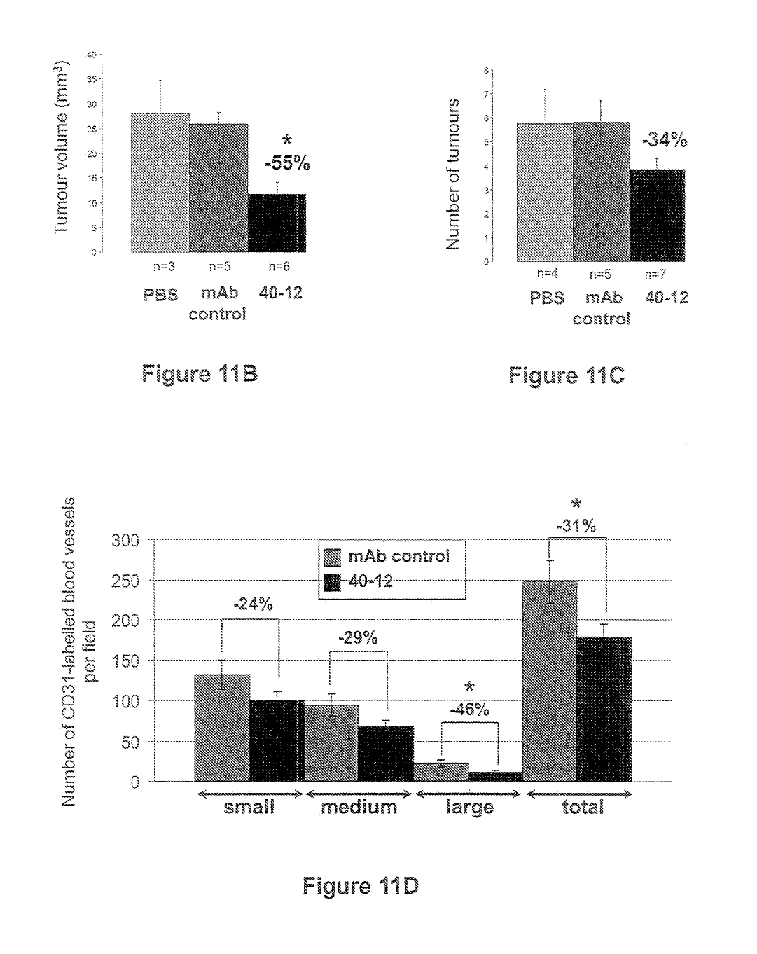

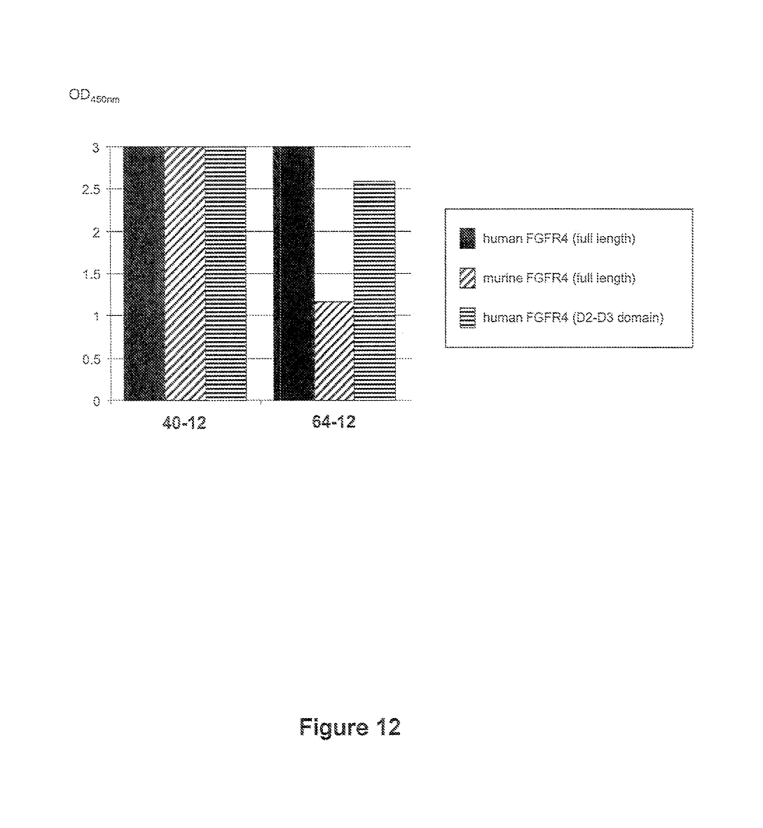

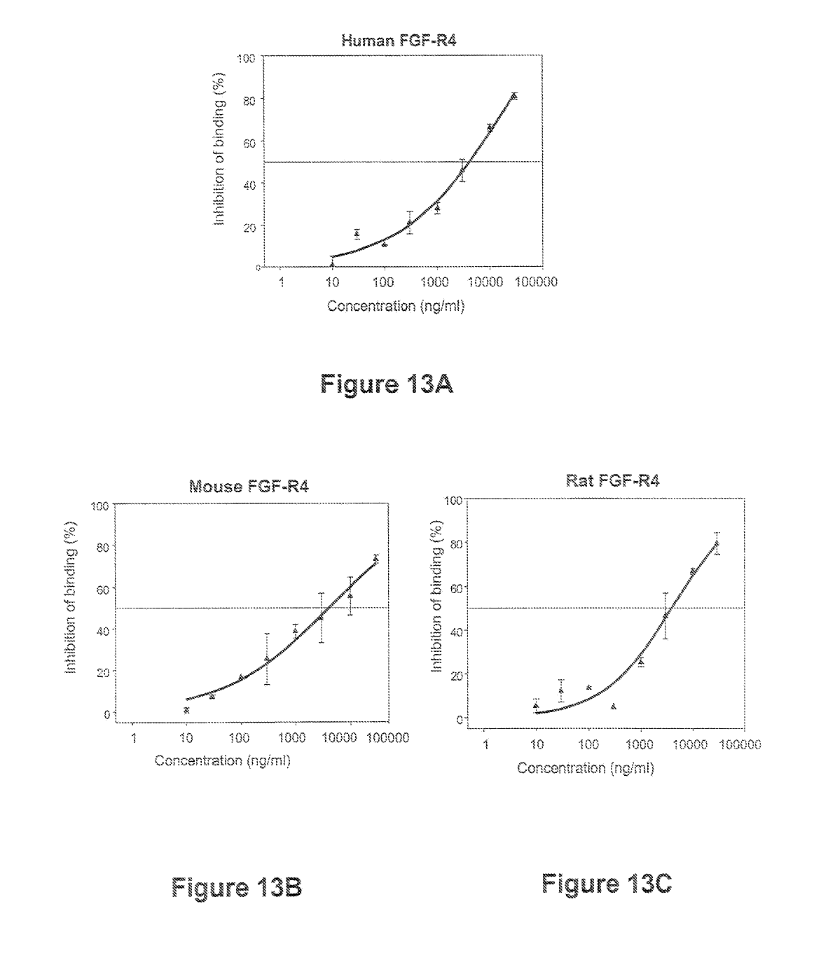

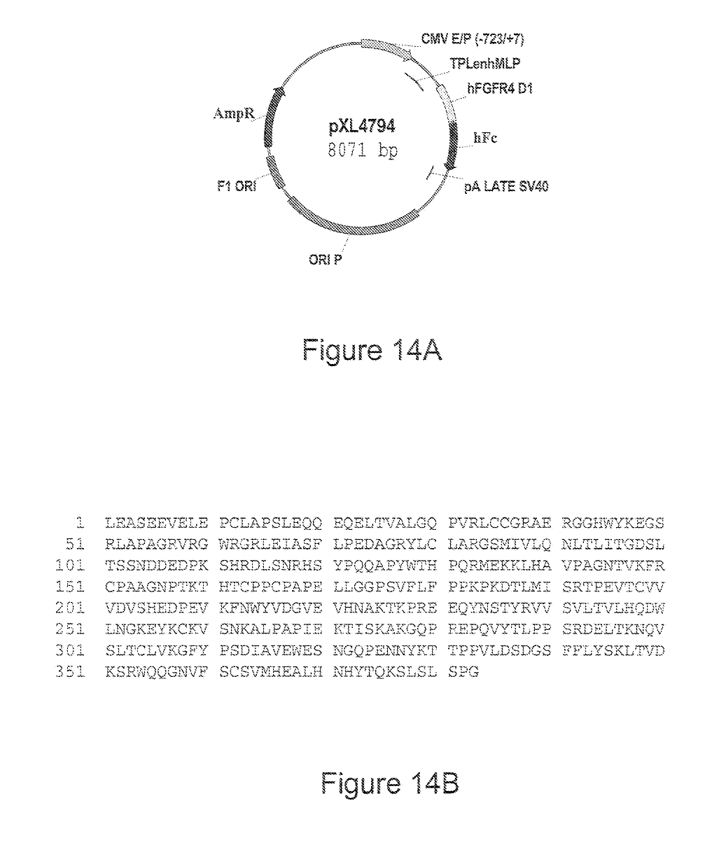

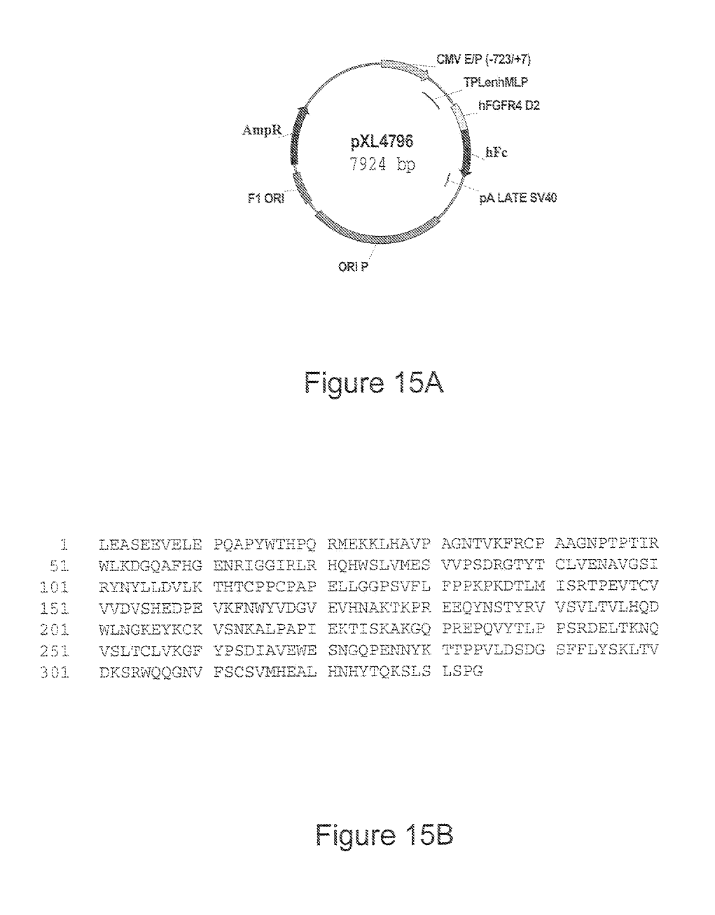

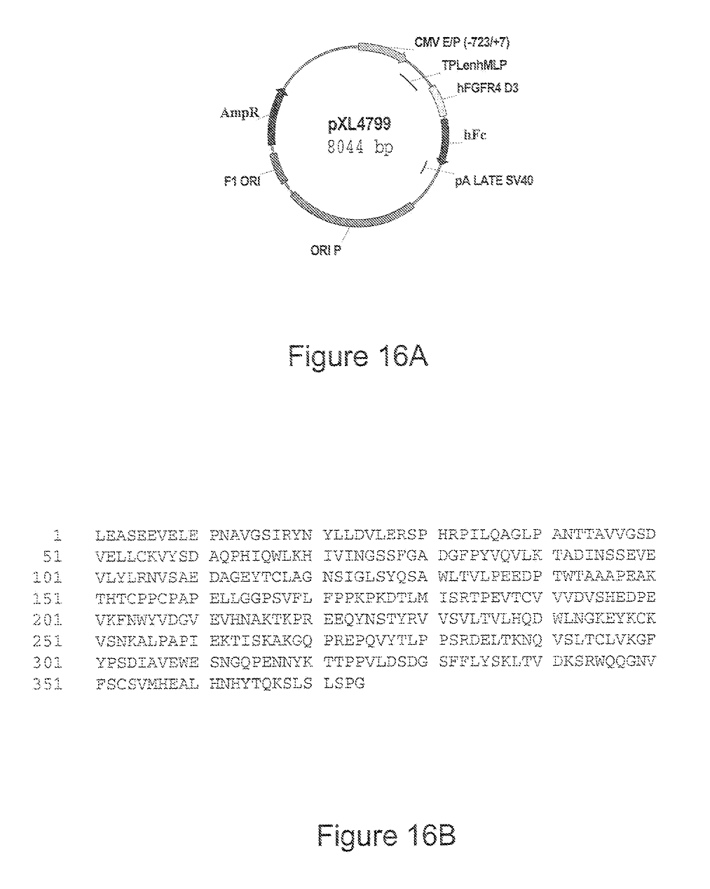

These Rip1-Tag2 mice are treated subcutaneously once a week, between weeks 10 and 13, at the dose of 25 mg/kg, with the antibody 40-12 or with the control antibody. After 13 weeks, the mice are sacrificed. The tumour volume and also the number of tumours per pancreas and the vascular density are determined. The treatment with the anti-FGFR4 antibody 40-12 makes it possible to significantly reduce the tumour volume by 55%, whereas the control antibody shows no effect (FIG. 11B). The antibody 40-12 also makes it possible to reduce by 34% the number of tumours per pancreas, compared with the control (FIG. 11C). This reduction in tumour volume is accompanied by a reduction in the vascular density, whether in terms of the number of small, medium or large vessels (FIG. 11D).

FIG. 12: The ability of the antibodies 40-12 and 64-12 to recognize the complete extracellular domain of human or murine FGF-R4, and also the extracellular domain of FGF-R4 deleted of its D1 domain, is measured by ELISA. Clone 40-12 is capable of binding equally with the 3 FGF-R4 constructs, whereas clone 64-12 recognizes the murine form of FGF-R4 less well.

FIGS. 13A to 13C: The antagonistic effect of the anti-FGFR4 antibody clone 40-12 on FGF2/FGF-R4 binding is measured by means of competition binding experiments with FGF2 labelled with an AlexaFluor® 488 nm. Clone 40-12 is capable of blocking the binding of human (FIG. 13A), murine (FIG. 13B) and rat (FIG. 13C) FGF2 on murine 300-19 cells transfected with cDNA encoding the human, murine or rat forms of the FGF-R4 receptor, with the same effectiveness (3500, 4110 and 3940 ng/ml, i.e. 23, 27 and 26 nM for the human, murine or rat complexes, respectively).

FIG. 14A: Map of the plasmid pXL4794 enabling the expression of hFGFR4_D1: Fc.

FIG. 14B: Amino acid sequence of the hFGFR4_D1: Fc protein secreted in the HEK293 line transfected with the plasmid pXL4794.

FIG. 15A: Map of the plasmid pXL4796 enabling the expression of hFGFR4_D2: Fc.

FIG. 15B: Amino acid sequence of the hFGFR4_D2: Fc protein secreted in the HEK293 line transfected with the plasmid pXL4796.

FIG. 16A: Map of the plasmid pXL4799 enabling the expression of hFGFR4_D3: Fc.

FIG. 16B: Amino acid sequence of the hFGFR4_D3: Fc protein secreted in the HEK293 line transfected with the plasmid pXL4799.

### EXAMPLES

### Example 1

### Demonstration of the Role of FGF-R4 in the Control of Angiogenesis

In order to demonstrate the role of FGF-R4 in the control of angiogenesis, in vitro angiogenesis experiments were carried out with human endothelial cells of HUVEC type stimulated with several FGFs: FGF2, a ligand which activates most of the FGF receptors; FGF19, a ligand which specifically activates FGF-R4; and FGF4, a ligand which does not activate FGF-R4.

To do this, gels were prepared by distributing, into each well of a chamberslide (Biocoat Cellware collagen, type I, 8-well culture slides: Becton dickinson 354630), 160 p1 of matrigel diluted to 1/6 (Growth factor reduced Matrigel: Becton dickinson 356230) in collagen (rat Tail collagen, type I: Becton dickinson 354236). The gels are maintained at 37° C. for 1 hour so as to enable them to polymerize. Next, the human vein endothelial cells (HUVEC ref: C-12200—Promocell) were seeded at 15×10³ cells/well in 400 μl of EBM medium (Clonetics C3121)+2% FBS+10 μg/ml of hEGF. This protocol can be adapted to 96-well plates: 60 μl per well of 96-well plates (Biocoat collagenl cellware, Becton Dickinson 354407). The matrix is prepared by mixing 1/3 of matrigel, 1 mg/ml final concentration of collagen, NaOH (0.026× the volume of collagen in μl), 1×PBS, the volume then being adjusted with water. The endothelial cells are stimulated with 1 ng/ml of FGF2 (R&D, 133-FB-025) or 10 ng/ml of FGF4 (R&D, 235-F4-025) or of FGF19 (R&D, 969-FG-025) for 24 h at 37° C. in the presence of 5% CO₂. After 24 hours, the length of the network of microtubules formed is measured using a computer-assisted image analysis system (Imagenia Biocom, Courtaboeuf, France) and the total length of the pseudotubules in each well is determined. The mean of the total length of the microcapillary network is calculated in μm for each condition corresponding to the mean of 6 replicates.

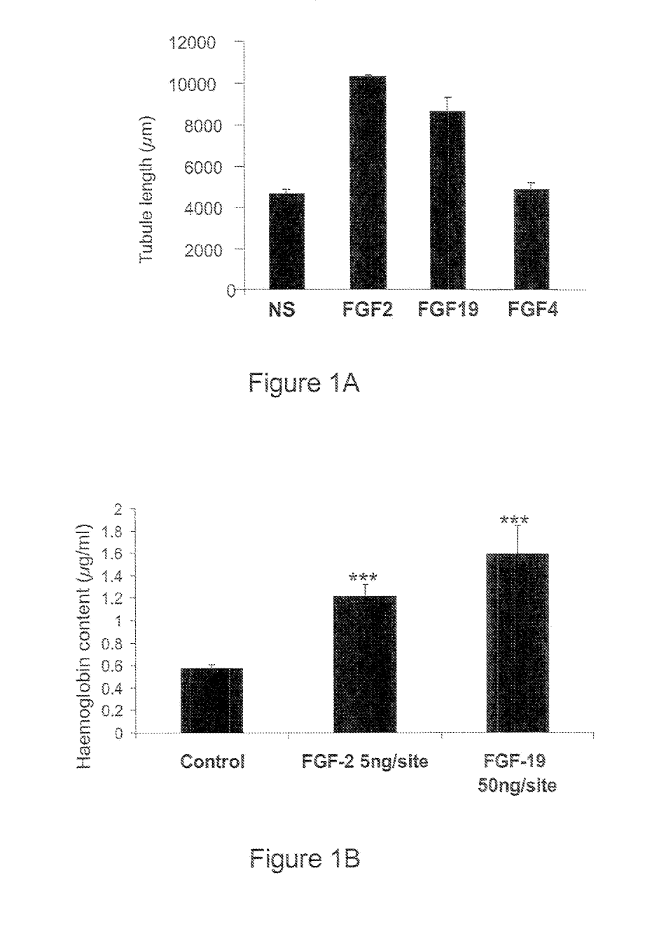

The stimulation with FGF2 or FGF19 enables the induction of the formation of new tubules, whereas FGF4 has no effect (FIG. 1A). These results show that the specific activation of FGF-R4 makes it possible to induce the formation of neovessels and therefore to conclude that FGF-R4 controls angiogenesis in vitro.

The in vivo correlation with the in vitro data was obtained using the model of angiogenesis induction in vivo in cellulose implants in mice. This model is an adaptation of the model described by Andrade and al. (Microvascular Research, 1997, 54, 253-61).

The animals, inbred white BALB/c J mice, are anaesthetized with a xylazine (Rompun®, 10 mg/kg)/ketamine (Imalgene 1000, 100 mg/kg) mixture given intraperitoneally. The back of the animal is shaved and disinfected with Hexomedine®. A pocket of air is created subcutaneously on the back of the mouse by injection of 5 ml of sterile air. An incision of approximately 2 cm is made on the upper back of the animal in order to introduce a sterile cellulose implant (disc 1 cm in diameter, 2 mm thick, Cellspon® ref 0501) impregnated with 50 μl of sterile solution containing the protein or the product to be tested. The incision is then sutured and cleaned with Hexomedine®. During the days following the implantation of the implant, the mice received, in the implant, the protein or the product by means of an injection through the skin (50 μl/implant/day) under gas anaesthesia (5% isoflurane (Aerrane®, Baxter)).

Seven days after the implantation of the sponge, the mice are sacrificed by means of a lethal dose of sodium pentobarbital (CEVA sante animale), administered intraperitoneally. The skin is then cut, approximately 1 cm around the sponge, avoiding the scar, in order to free the skin and the sponge. The sponge is then cut up into several pieces and placed in a Ribolyser® tube containing 1 ml of lysis buffer (Cell Death Detection ELISA, Roche). The tubes are shaken 4 consecutive times, for 20 sec, force 4, in a cell homogenizer (FastPrep® FP 120). The tubes are then centrifuged for 10 min at 2000 g at 20° C. and the supernatants are frozen at −20° C. while awaiting the haemoglobin assay.

On the day of the assay, the tubes are again centrifuged after thawing, and the haemoglobin concentration is measured with the Drabkin reagent (Sigma, volume for volume) by reading on a spectrophotometer at 405 nm against a standard range of bovine haemoglobin (Sigma). The haemoglobin concentration in each sample is expressed in mg/ml according to the polynomial regression performed on the basis of the standard range. The results are expressed in mean value (±sem) for each group. The differences between the groups are tested with an ANOVA followed by a Dunnett's test on the square root of the values.

In this model, FGF19 at 50 ng per site and 5 re-injections is capable of significantly inducing colonization of the sponge by newly formed mature vessels, with the same effectiveness as FGF2 at 5 ng per site. The presence of functional blood vessels in the sponge is demonstrated by the presence of haemoglobin (FIG. 1B). These results show that the specific activation of FGF-R4 enables the recruitment of functional blood vessels, indicating that FGF-R4 also controls angiogenesis in vivo.

### Example 2

### Description of the FGFR Proteins Used as Immunogen and Antigen

FGFR growth factor receptors, and in particular the extracellular domains of FGF-R1, FGF-R2, FGF-R3, FGF-R4, are fused to a tag (Histag) or to the immunoglobulin Fc domain.

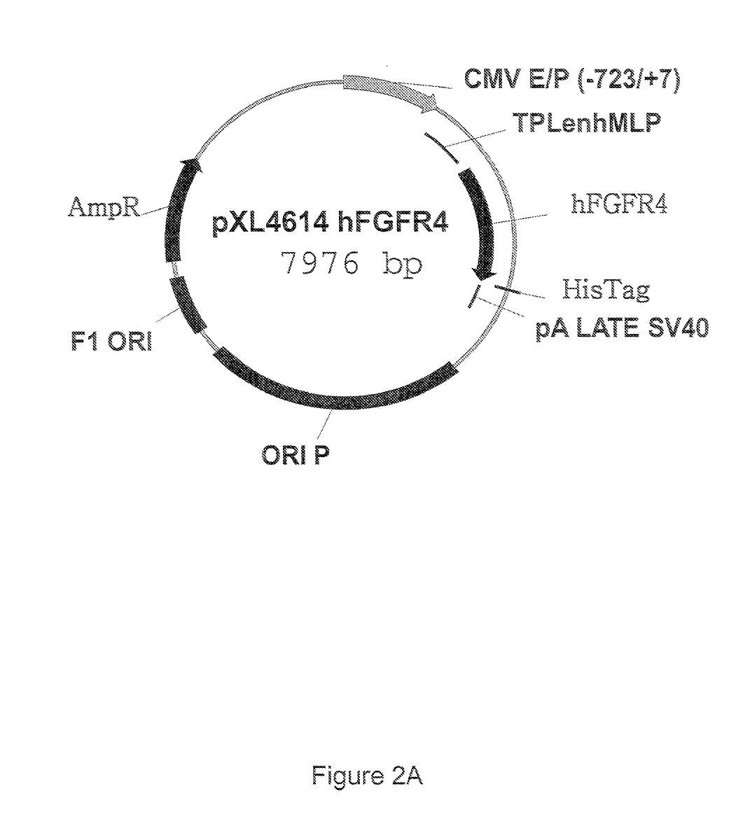

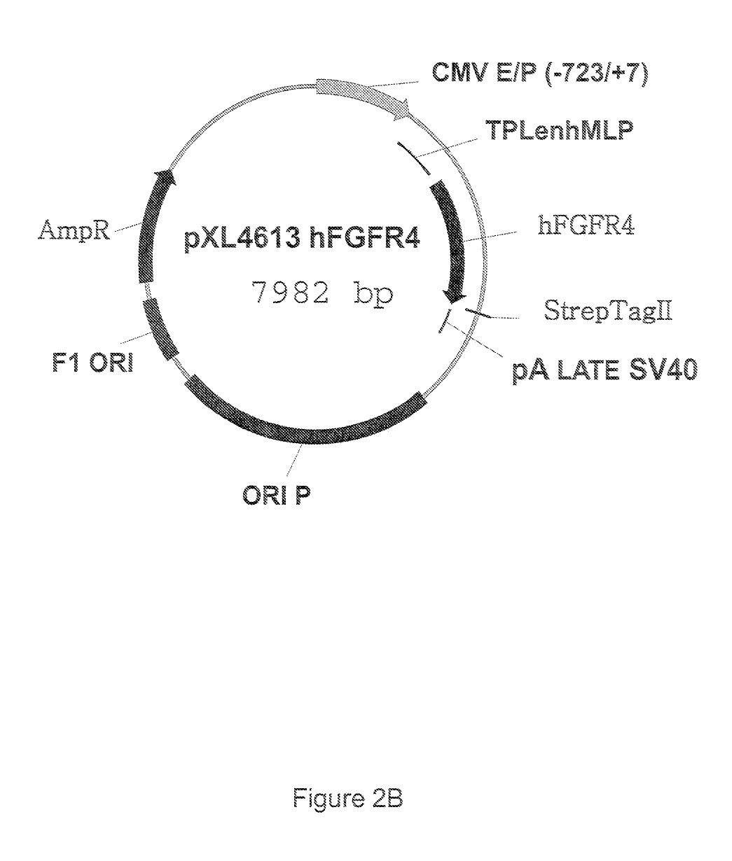

The cDNA encoding the extracellular domain of human FGF-R4 corresponds to the protein described in SwissProt FGF-R4_HUMAN position 1-365 with the L136P mutation. It was cloned into the eukaryotic expression vector pXL4614 represented in FIG. 2, in order to express a protein containing a Histag in the C-terminal position in the extracellular domain.

The proteins named hFGFR4-Histag, of sequence SEQ ID No. 40, were produced by transient transfection in the HEK293 EBNA line (Invitrogen) using the plasmid pXL4614 and the helper plasmids pXL4544 and pXL4551 which enable the expression of two N-glycan glycosylation enzymes, i.e. α-2,3-sialyltransferase and β-1,4-galactosyltransferase, as described in application WO2008/065543.

The hFGFR4-Histag protein expressed in the HEK293 EBNA cell culture supernatant was purified by chromatography on an Ni-chelating sepharose column (Amersham Biosciences; ref. 17-0575-01), elution being carried out in an imidazole buffer, and then formulated in PBS buffer (Invitrogen; ref. 14190-094). The analysis of the monosaccharide composition and the quantification of the sialic acids of the N-glycans, as described by Saddic and al. 2002. (Methods Mol. Biol. 194:23-36 and Anumula and al. 1998. Glycobiology 8:685-694), made it possible to demonstrate that the protein was very highly sialylated (91%). Consequently, the protein had all the characteristics for having sufficient pharmacokinetic properties.

In a comparable manner, the hFGFR4-Streptag protein (SEQ ID No. 69) was purified by chromatography on a Strep-Tactin Superflow column (IBA; ref. 2-1206), elution being carried out in a desthiobiotin buffer, and then formulated in PBS buffer.

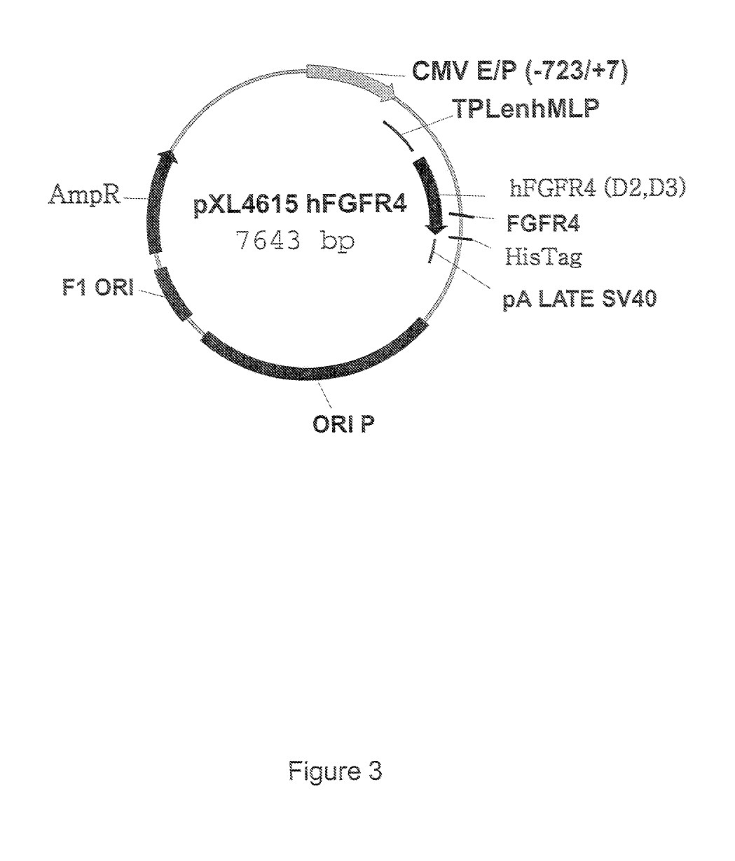

The hFGFR4(D2,D3)-Histag protein corresponds to the sequence SEQ ID No. 42. The cDNA was cloned into the eukaryotic expression plasmid pXL4615 represented in FIG. 3, and the protein was produced and purified under conditions comparable to the hFGFR4-Histag protein.

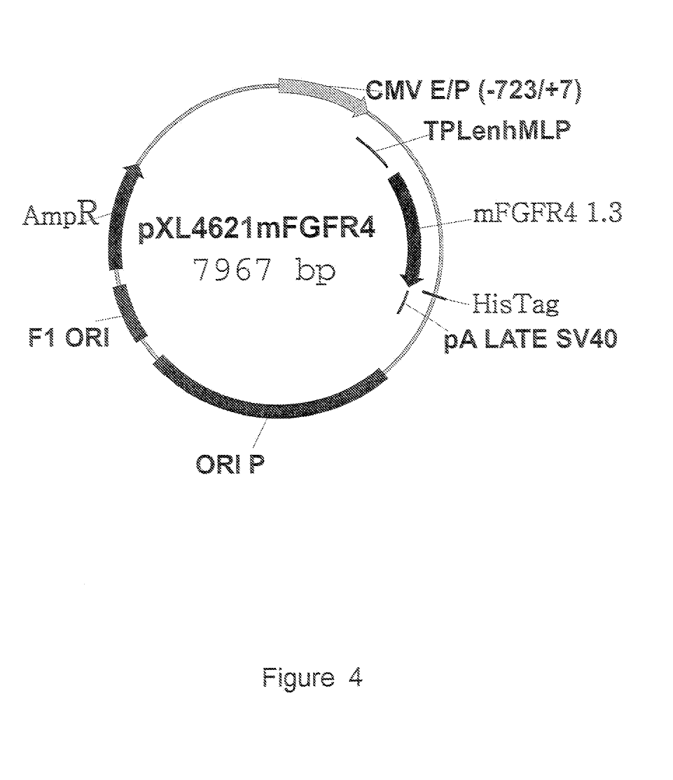

The cDNA encoding the mFGFR4-Histag protein (SEQ ID No. 43) was cloned into the eukaryotic expression plasmid pXL4621 represented in FIG. 4, and the protein was produced and purified under conditions comparable to the hFGFR4-Histag protein.

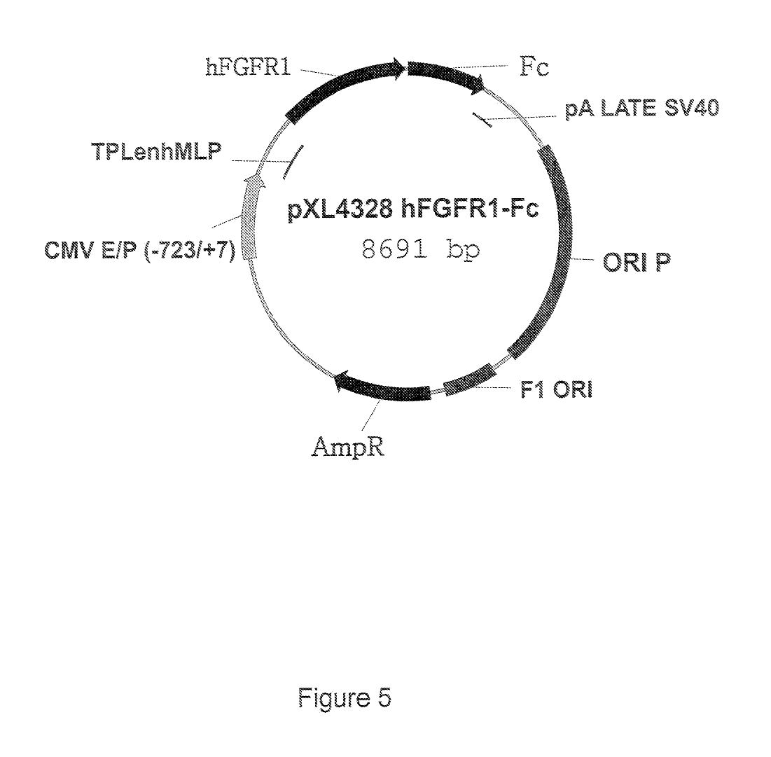

The hFGFR1-Fc protein (SEQ ID No. 46) contains the extracellular domain of human FGF-R1 IIIc fused, in the C-terminal position, to the Fc domain of human IgG1. The cDNA was cloned into the eukaryotic expression plasmid pXL4728 represented in FIG. 5, and the protein was produced under conditions comparable to the hFGFR4-Histag protein and then purified by chromatography on a protein G Sepharose affinity column (Amersham Biosciences), elution being carried out in 100 mM glycine/HCl buffer, pH 2.7, and then formulated in PBS buffer.

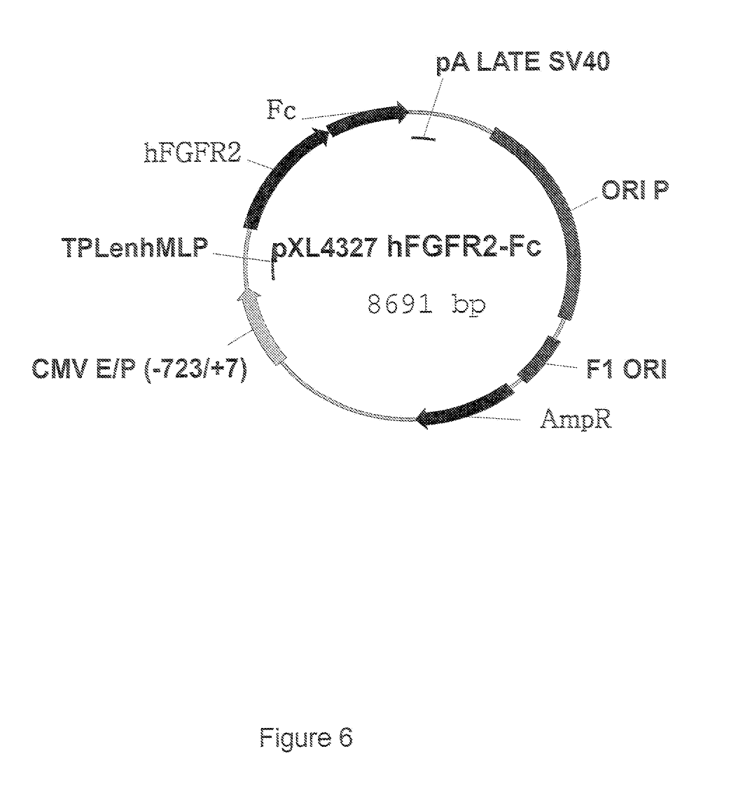

The hFGFR2-Fc protein, of sequence SEQ ID No. 48, contains the extracellular domain of human FGF-R2 IIIc fused, in the C-terminal position, to the Fc domain of human IgG1. The cDNA was cloned into the eukaryotic expression plasmid pXL4327 represented in FIG. 6, and the protein was produced and then purified under conditions comparable to the hFGFR1-Fc protein.

The hFGFR3-Fc protein is a protein which fuses the extracellular domain of human FGF-R3 IIIc to the Fc domain of human IgG1, and was obtained from R&D Systems (ref: 760-FR).

The hFGFR4-Fc protein is a protein which fuses the extracellular domain of human FGF-R4 to the Fc domain of human IgG1, and was obtained from R&D Systems (ref: 685-FR).

Various subdomains of the extracellular portion of the hFGFR4 protein were fused to the Fc domain of human IgG1. The D1 subdomain is contained in the construct SABVA4794 (SEQ ID No. 112 and FIGS. 14A and 14B). The D2 subdomain is contained in the construct SABVA4796 (SEQ ID No. 114 and FIGS. 15A and 15B). The D3 subdomain is contained in the construct SABVA4799 (SEQ ID No. 116 and FIGS. 16A and 16B). These three subdomains extend respectively from positions 1 to 179 for SABVA4794, 1 to 32 plus 145 to 242 for SABVA4796, and 1 to 32 plus 228 to 360 for SABVA4799 (positions described in SwissProt FGF-R4_HUMAN). These were produced using the plasmids pXL4794 (coding sequence SEQ ID No. 111), pXL4796 (coding sequence SEQ ID No. 113) and pXL4799 (coding sequence SEQ ID No. 115) under conditions comparable to the FGFR1-Fc protein.

### Example 3

### Generation and Screening of Anti-FGF-R4 Monoclonal Antibodies

### A—Antibodies Obtained by Immunization

The monoclonal antibodies were obtained by immunization with the hFGFR4-Histag immunogen in five BALB/cJ mice (Charles River), 6 to 8 weeks old, each immunized with a total of 24 μg of hFGFR4-Histag by the RIMMS method described by Kilpatrick and al. (1997. Hybridoma 16: 381389) and the fusion protocol described in ClonaCell™-HY Hybridoma Cloning Kit (StemCell Technologies; ref 03800).

Two days after the final injection, the mice were sacrificed and the lymph nodes were fused with P3×63-AG8.653 myeloma cells (ATCC, CRL-1580) in a 5:1 ratio in the presence of polyethylene glycol (ClonaCell™-HY ref. 03806). The cell suspension was distributed aseptically into Petri dishes incubated at 37° C. in the presence of 5% CO₂. The colonies that had appeared after 12 days of incubation were isolated and cultured in medium E (ClonaCell™-HY; ref. 03805) in 96-well plates.

The primary screening of monoclonal antibodies obtained by immunization with hFGFR4-Histag was carried out by ELISA assay using hFGFR4-Streptag as capture antigen. The capture antigen was bound to Immulon-4 enzyme-linked plates (VWR Scientific Inc. Swedesboro, N.J.). The hybridoma culture supernatants were subsequently added and then detection was carried out using the peroxidase-conjugated anti-mouse IgG rabbit antibody (Sigma; ref. A9044-dilution to 1:50 000). The revealing was carried out with the TMB-H2O2 substrate (Interchim; ref UP664780) and the measurements were carried out with the plate reader at 450 nm. Among the 444 hybridomas tested, 129 were positive by ELISA assay with the hFGFR4-Streptag antigen, and 120 of these hybridomas were also positive with the hFGFR4-Fc dimer protein.

A secondary screen was carried out, in order to select only the FGF-R4-specific antibodies, by ELISA assay using as capture antigen the hFGFR4-Streptag protein, and then with the hFGFR1-Fc, hFGFR2-Fc and hFGFR3-Fc proteins described in Example 2. The capture antigen was bound to Immulon-4 enzyme-linked plates (VWR Scientific Inc. Swedesboro, N.J.). The hybridoma culture supernatants were subsequently added and then detection was carried out using the peroxidase-conjugated anti-mouse IgG rabbit antibody (Sigma; ref. A9044-dilution to 1:50 000). The revealing was carried out with the TMB-H2O2 substrate (Interchim; ref UP664780) and the measurements were carried out with the plate reader at 450 nm. Among the 129 hybridomas that tested positive by ELISA assay with the antigen, 84 hybridomas were positive with hFGFR4-Streptag and had no affinity for either hFGFR1-Fc, hFGFR2-Fc or hFGFR3-Fc. 39 hybridomas were conserved, as a function of their growth and their morphology. Their isotype was determined using the SEROTEC kit (ref. MMT1); 95% were IgG1s.

A tertiary screen was carried out on a test for FGF2-induced proliferation of Baf/3 modified cells, in order to characterize the inhibition by the anti-FGFR4 antibodies.

The murine hybridomas expressing the anti-FGFR4 antagonist antibodies were cloned by limiting dilution. Using the hybridoma cells cultured in the exponential phase, the coding sequence (cDNA) was determined after extraction of the mRNA using the Oligotex kit (Qiagen; ref 72022); production and amplification of the cDNA by the RACE-RT method with the Gene Racer kit, the SuperScript III reverse transcriptase (Invitrogen; ref L1500) and the primers described in Table 2 below; amplification of the cDNA fragments using the Phusion polymerase (Finnzymes; ref. F-5305), the primers and the temperature conditions described in Table 2. The amplified fragments containing the coding regions for VH (variable region of the heavy chain HC) or VL (variable region of the light chain LC) were cloned into the pGEM-T Easy vector from Promega; ref A137A, and the inserts of the plasmids obtained were sequenced, such that the coding sequence of each variable domain was analysed in the 5′-3′ and 3′-5′ direction on at least 6 plasmids corresponding to the anti-FGFR4 antibody 40-12 and the anti-FGFR4 antibody 64-12. The analyses of sequence, contigs and alignments were carried out using the software available on Vector NTI (Invitrogen).

The plasmids containing the consensus sequences encoding the variable regions of the anti-FGFR4 antibodies were conserved. The plasmid pXL4691 contains the sequence encoding the VH of sequence SEQ ID No. 5 of the anti-FGFR4 antibody 40-12, and the plasmid pXL4693 contains the sequence encoding the VH of sequence SEQ ID No. 19 of the anti-FGFR4 antibody 64-12, as shown in Table 2 below.

The plasmid pXL4690 contains the nucleotide sequence SEQ ID No. 7 encoding the VL of sequence SEQ ID No. 8 of the anti-FGFR4 antibody 40-12, and the plasmid pXL4692 contains the nucleotide sequence SEQ ID No. 21 encoding the VL of sequence SEQ ID No. 22 of the anti-FGFR4 antibody 64-12, as shown in Table 2 below.

TABLE 2
Operating conditions and analysis of the
reverse transcriptase and PCR reactions-
identification of the plasmids pXL4690 to pXL4693.

| Anti-FGFR4 | 40-12 | 64-12 |
|---|---|---|
| RT reaction | 55° C. | 55° C. |
| Temperature | SEQ ID No. 64 (HC) | SEQ ID No. 64 (HC) |
| Primers | SEQ ID No. 65 (LC) | SEQ ID No. 66 & SEQ |
| | | ID No. 67 (LC) |
| 5′ Primer-GeneRacer | 5′-CGACTGGAGCACGAGGACACTGA-3′ | |
| (SEQ ID No. 63) | | |
| 3′ Primer-internal | 5′-TATGCAAGGCTTACAACCACA-3′ | |
| to murine hinge | | |
| (SEQ ID No. 64) | | |
| 3′ Primer-internal | 5′-CTCATTCCTGTT | |
| to murine C_{K} | GAAGCTCTT GAC-3′ | |
| (SEQ ID No. 65) | | |
| 3′ Primers-internal | | 5′-ACACTCAGCACGGG |
| to murine Cλ | | ACAAACTCTTCTC-3′ |
| (SEQ ID No. 66 and | | 5′-ACACTCTGCAGGAG |
| SEQ ID No. 67) | | ACAGACTCTTTTC-3′ |
| HC PCR: | 55° C. | 55° C. |
| Temperature | SEQ ID No. 63- | SEQ ID No. 63- |
| Primers | SEQ ID No. 64 | SEQ ID No. 64 |
| Sequence of the | 9 clones have the same | 6 clones have the same |
| cloned PCR | VH sequence and a CH1 | VH sequence and a CH1 |
| products (VH) | sequence identical | sequence identical |
| | to that of mCH1- | to that of mCH1 |
| Plasmid | pXL4691 | pXL4693 |
| containing VH | | |
| LC PCR: | 55° C. | 55° C. |
| Temperature | SEQ ID No. 63- | SEQ ID No. 63- |
| Primers | SEQ ID No. 65 | SEQ ID No. 66 & 67 |
| Sequence of the | 11 clones have | 6 clones have the |
| cloned PCR | the same VL sequence | same VL sequence |
| products (VL) | and a C_{K} sequence | and a Cλ sequence |
| | identical to mC_{K} | identical to mCλ |
| Plasmid | pXL4690 | pXL4692 |
| containing VL | | |

The amino acid sequences of the light and heavy variable regions, respectively, of the anti-FGFR4 antibody 64-12 and the anti-FGFR4 antibody 40-12 are different. The numbers of the sequences used, obtained and deduced are indicated in Table 7.

The antibodies 40-12 and 64-12 were produced in T500 flasks. The culture supernatant was harvested after 7 days. The anti-FGFR4 antibodies were affinity-purified on protein G and then dialysed against PBS, filtered sterile, and stored at 4° C.

The purified antagonist antibodies have a K_{D} of 6.5×10⁻⁹ M (anti-FGFR4 40-12) and 5.75×10⁻⁸ M (anti-FGFR464-12).

### B—Antibody Selected Using the Phage Display Method

The primary screening of monoclonal antibodies obtained by phage display with hFGFR4-Histag was carried out by ELISA assay using hFGFR4-Histag as capture antigen. The capture antigen was bound to Immulon-2 enzyme-linked plates (VWR Scientific Inc. Swedesboro, N.J.). The culture supernatants from E. coli infected with the phages were subsequently added and then detection was carried out using the peroxidase-conjugated anti-M13 mouse antibody (GE Healthcare; ref. 27-9421-01, dilution to 1:5000). The revealing was carried out with the TMB-H2O2 substrate (Interchim; ref UP664780) and the optical density (O.D.) measurements were carried out at 450 nm.

A secondary screen was carried out in order to select only the FGF-R4-specific antibodies, by ELISA assay using as capture antigen the hFGFR4-Histag protein, and then with the hFGFR1-Fc, hFGFR2-Fc and hFGFR3-Fc proteins described in Example 2. The capture antigen was bound to Immulon-2 enzyme-linked plates (VWR Scientific Inc. Swedesboro, N.J.). The culture supernatants from E. coli infected with the phages were subsequently added and then detection was carried out using the peroxidase-conjugated anti-M13 mouse antibody (GE Healthcare; ref. 27-9421-01, dilution to 1:5000). The revealing was carried out with the TMB-H2O2 substrate (Interchim; ref UP664780) and the optical density (O.D.) measurements were carried out at 450 nm.

The FGF-R4-specific clones selected were sequenced and recloned into an expression vector for transient transfection of HEK293 cells.

For this, in a first step, the regions encoding Fab, i.e. the light chain of the antibody, a bacterial ribosome binding site, and the heavy chain variable region of the antibody, are extracted from the phagemid by restriction and inserted into an IgG expression plasmid for mammalian cells, downstream of a eukaryotic antibody signal sequence and upstream of the constant region of a human IgG1 heavy chain. In a second step, the region containing the bacterial ribosome binding site and the bacterial signal peptide for the heavy chain is exchanged against an IRES sequence and a eukaryotic signal sequence. The IgGs are expressed by transient transfection of HEK293 cells. This process is described in detail in T. Jostock et al., Journal of Immunological Methods 289 (2004) 65-80.

An example of a human IgG1 constant region sequence that can be used in the present invention is the sequence SEQ ID No. 117.

A tertiary screen was carried out on a test for FGF2-induced proliferation of modified Baf/3 cells, described in Example 4, in order to characterize the inhibition by the anti-FGFR4 antibodies, using the culture supernatants from HEK293 cells transiently transfected with the antibody expression vectors. This screen made it possible to identify the antibodies of clones 8, 31, 33 and 36. The corresponding sequences are described in Table 7.

### Example 4

### Establishment of the BaF/3 FGF-R4-hMpI Murine Clonal Line and Cell Proliferation Protocol

The extracellular and transmembrane domain of FGF-R4 was cloned, as a translational fusion with the intracellular domain of hMpI, into a mutated pEF6N5-His A vector in order to obtain the presence, in the 5′ position, of the HA tag in front of the chimeric FGF-R4-hMpI receptor.

Construction of the Mutated pEF6A

The pEF6/V5-His A vector (Invitrogen, reference V961-20) was improved in order to integrate the MCS (multi cloning site) associated with the HA tag placed under the signal peptide of IgGk of pDisplay (Invitrogen, reference V660-20). To do this, the MCS associated with the HA tag and with its signal peptide were amplified by PCR between the sense primer of sequence SEQ ID No. 51 and the reverse primer of sequence SEQ ID No. 52, making it possible to insert the KpnI restriction site in the 5′ position and the XbaI restriction site in the 3′ position. The PCR fragment was digested with the KpnI and XbaI enzymes and then cloned into the pEF6/V5-His A vector opened with the same enzymes. Finally, the first BamHI site of the MCS was replaced with the BsrGI site by digesting the newly formed vector with the KpnI and SpeI enzymes and inserting, between these sites, the primers of sequences SEQ ID No. 53 and SEQ ID No. 54, hybridized to one another and containing the BsrGI enzyme site. The vector obtained was called: pEF6mut-HA.

Construction of the pEF6mut-HA-FGF-R4-hMpI Vector

The intracellular domain of MpI was amplified in a vector pEF6/V5-His TOPO (Invitrogen, reference K9610-20) between the sense primer of sequence SEQ ID No. 55 allowing the insertion of the SacI digestion site and the reverse primer of sequence SEQ ID No. 56, commonly called revBGH. The PCR fragments generated were then digested with the SacI and NotI enzymes.

The extracellular and transmembrane domain of FGF-R4 was amplified using the pair of primers (sense: sequence SEQ ID No. 57; reverse: sequence SEQ ID No. 58). These primers make it possible to insert the BamHI enzymatic site in the 5′ position and the SacI enzymatic site in the 3′ position. The PCR fragment obtained was then digested with the BamHI and SacI enzymes.

The amplifications of DNA encoding hMpI and encoding FGF-R4 were then cloned simultaneously into the pEF6mut-HA vector opened with BamHI-NotI. The resulting construct was called “pEF6mut-HA FGF-R4αIIIc-hMpI2”. This construct was then improved by site-directed mutagenesis in order to change the transmembrane domain of FGF-R4 (protein sequence SEQ ID No. 59) for the sequence SEQ ID No. 60. To do this, the “QuickChange® Site-directed mutagenesis kit” (Clontech, reference 200518) was used with the sense primer of sequence SEQ ID No. 61 and the reverse primer of sequence SEQ ID No. 62. The new chimeric construct obtained was called pEF6mut-HA FGF-R4αIIIcmut-hMpI2.

Creation of a Stable Line by Transfection of the BaF/3 Murine Line with the FGF-R4αIIIcmut-hMpI2 Construct:

The “pEF6mut-HA FGF-R4αIIIIcmut-hMpI2” construct was stably introduced, by electroporation, into the genome of the BaF/3 murine cells. The line obtained was selected in the presence of FGF2 at 20 ng/ml (R&D, reference 234-FSE-025) and of heparin at 100 ng/ml (Sigma, reference H3149). The transfected and selected line is then of clonal type.

Cell Proliferation Protocol with the BaF/3 FGFR4-hMpI Cell Line:

The BaF/3 FGFR4-hMpI cells were cultured and maintained in complete RPMI 1640 medium (Invitrogen; ref. 32404-014) (10% FCS (Hyclone; ref. SH30070.03), 2 mM glutamine, 1×MEM non essential amino acid (Gibco, ref 11140-035), 1×MEM sodium pyruvate (Gibco, ref 11360-039), supplemented with FGF2 at 20 ng/ml (R&D Systems, ref 234-FSE) and heparin at 3 ng/ml (Sigma, ref H3149). On day one, the cells are seeded at 0.4×10⁶ cells/ml in complete RPMI 1640 medium supplemented wuith FGF2 at 20 ng/ml and with heparin at 3 ng/ml. The following day, 50 μl of BaF/3 FGFR4-hMpI cell suspension in complete RPMI 1640 medium supplemented with FGF2 at 20 ng/ml and with heparin at 3 ng/ml, at 0.2×10⁶ cells/ml, were dispensed into 96-well plates (Porvair, ref 214006), followed by 50 μl of hybridoma supernatant containing the antibody to be tested. The plates were then placed at 37° C., 5% CO₂ for 24 to 30 h. For reading the cell proliferation, the amount of ATP was quantified by adding 100 μl of Cell Titer Glo Luminesent Cell Viability Assay (Promega, ref G7571) and the luminescence was read using a luminometer.

The clones exhibiting, in this test, a signal 50% weaker than that of the complete RPMI 1640 medium containing the additives FGF2 at 20 ng/ml and heparin at 3 ng/ml were selected.

When the anti-FGFR4 antibodies, specific for FGF-R4, were incubated with the Baf/3-FGFR4-hMpI cells and with the following additives FGF2 at 20 ng/ml and heparin at 3 ng/ml, an antagonist effect was observed on cell proliferation. Among the 39 hybridomas tested, 14 are capable of inhibiting FGF-induced cell proliferation of Baf/3-FGFR4-hMpI cells in the presence of FGF2.

It was shown by ELISA (FIG. 12) that the anti-FGFR4 antagonist antibodies 40-12 and 64-12 had affinity for the murine protein mFGFR4 and human protein hFGFR4 (D2, D3).

Finally, a last screen was carried out by Surface Plasmon Resonance (BIAcore 2000) in order to determine the affinity constants of the anti-FGFR4 antibodies. The interaction between the FGF-R4 protein and the anti-FGFR4 antibody present in the hybridoma culture supernatant was analysed after having bound the anti-FGFR4 antibody to an anti-Fc antibody, itself bound to the CM chip. The kinetics are measured according to the protocol of Canziani et a/2004. Anal. Biochem. 325: 301-307.

Two antibodies among those having affinity constants of 10⁻⁵ to 10⁻⁹ M and dissociation rates of 10⁴ to 10⁻⁵ s⁻¹ were selected. The characteristics of these antibodies are described in Table 1 below.

The reference method used to determine the K_{D} is Surface Plasmon Resonance (BIAcore).

TABLE 1A
Affinity and association/dissociation constants of the purified
anti-FGFR4 antagonist antibodies 40-12 and 64-12

| Anti-FGFR4 | kₒₙ (M⁻¹ · s⁻¹) | k_{off} (s⁻¹) | K_{D} (M) |
|---|---|---|---|
| 40-12 | 1.94 × 10⁵ | 1.26 × 10⁻³ | 6.50 × 10⁻⁹ |
| 64-12 | 1.05 × 10⁴ | 6.02 × 10⁻⁴ | 5.75 × 10⁻⁸ |

TABLE 1B
Kinetic parameters for binding of the anti-FGFR4 antibodies 8, 31,
33 and 36, measured by Surface Plasmon Resonance (BIAcore 3000):

| | on h-FGFR4-Histag | | | on m-FGFR4-Histag | | |
|---|---|---|---|---|---|---|
| | Kon (M⁻¹s⁻¹) | Koff (s⁻¹) | KD (M) | Kon (M⁻¹s⁻¹) | Koff (s⁻¹) | KD (M) |
| Clone 8 | 8.93 × 10⁵ | 2.92 × 10⁻⁴ | 3.27 × 10⁻¹⁰ | 1.20 × 10⁶ | 1.36 × 10⁻⁴ | 1.15 × 10⁻¹⁰ |
| Clone 31 | 6.48 × 10⁵ | 9.80 × 10⁻⁴ | 1.52 × 10⁻⁹ | 9.14 × 10⁵ | 1.80 × 10⁻³ | 1.97 × 10⁻⁹ |
| Clone 33 | 8.05 × 10⁵ | 6.17 × 10⁻⁴ | 7.71 × 10⁻¹⁰ | 1.01 × 10⁶ | 7.04 × 10⁻⁴ | 6.92 × 10⁻¹⁰ |
| Clone 36 | 2.44 × 10⁵ | 6.31 × 10⁻⁴ | 2.62 × 10⁻⁹ | 3.30 × 10⁵ | 7.52 × 10⁻⁴ | 2.28 × 10⁻⁹ |

### Example 5

### Specificity of the Anti-FGFR4 Antibodies

### A—Specificity of the Anti-FGFR4 Antibody 40-12 for FGF-R4

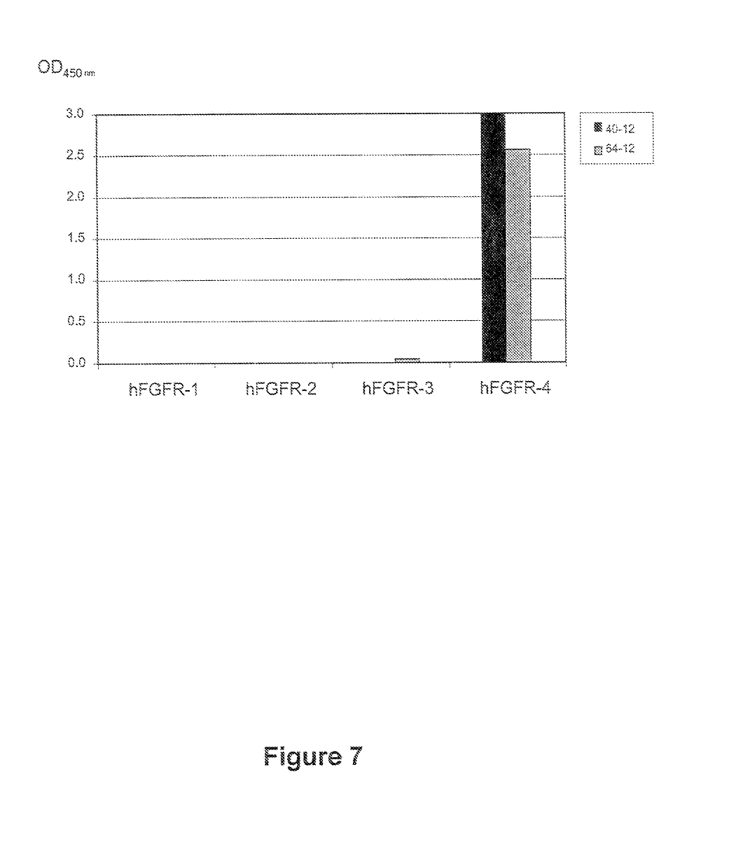

The specificity of each antibody is established by ELISA according to the protocol described in Example 4. In this way, the ability of each antibody to bind to each FGF-R is observed. This experiment clearly shows that the antibody 40-12 recognizes only FGF-R4 and is therefore specific for FGF-R4. The antibody 64-12 is capable of binding mainly to FGF-R4, but also weakly to FGF-R3 (FIG. 7).

### B—Specificity of the Anti-FGFR4 Antibodies 8, 31, 33 and 36 for FGF-R4

The specificity of each antibody is established by ELISA according to the following protocol: suspensions of phages displaying, at their surface, the antibodies in the Fab format are generated by infection of E. coli bacteria. The capture antigens were bound to Immulon-2 enzyme-linked dishes (VWR Scientific Inc. Swedesboro, N.J.). The phage suspensions were subsequently added and then detection was carried out using the peroxidase-conjugated anti-M13 phage mouse antibody (GE Healthcare, ref. 27-9421-01, dilution to 1:5000). The revealing was carried out with the TMB-H2O2 substrate (Interchim; ref UP664780) and the optical density (OD) measurements were carried out at 450 nm. Table 2 summarizes the results obtained:

In this way, the ability of each antibody to bind to each FGF-R is observed. This experiment clearly shows that the antibodies 8, 31, 33, 36 recognize only FGF-R4 and are therefore specific for FGF-R4.

TABLE 2
Specificity of the anti-FGF-R4 antibodies derived from clones
8, 31, 33 and 36, established by ELISA (Signal = OD, 450 nm)

| | h-FGFR4(D2,D3)- | h-FGFR1- | h-FGFR2- | h-FGFR3- |
|---|---|---|---|---|
| | histag | Fc | Fc | Fc |
| Clone 8 | 2.55 | 0.05 | 0.00 | 0.00 |
| Clone 31 | 2.52 | 0.00 | 0.00 | 0.01 |
| Clone 33 | 2.73 | 0.00 | 0.00 | −0.01 |
| Clone 36 | 1.87 | −0.01 | −0.01 | −0.02 |

### Example 6

### Antagonistic Effect of the Antibody 40-12 and of the Antibodies Derived from Clones 8, 31, 33, 36, on Angiogenesis (In Vitro)

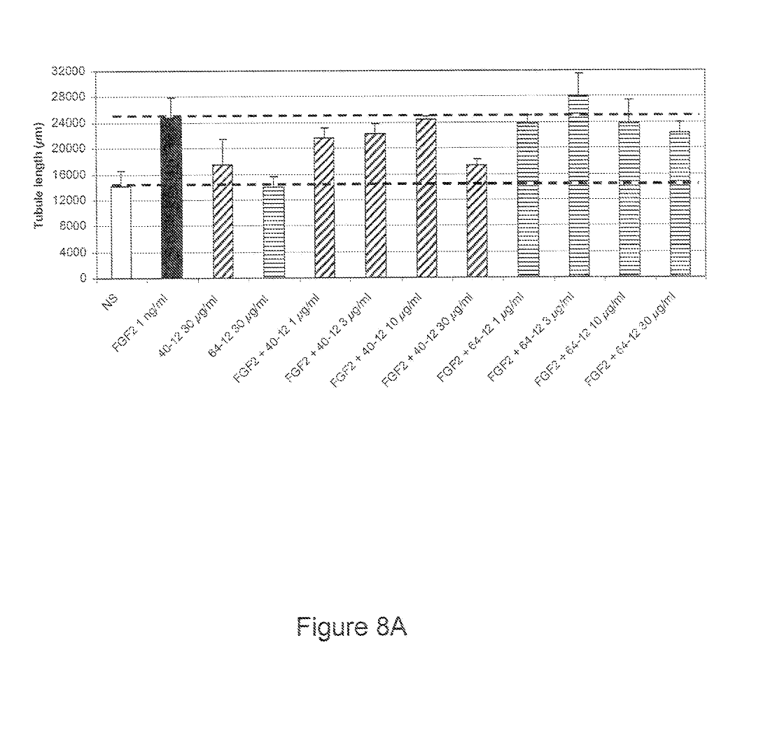

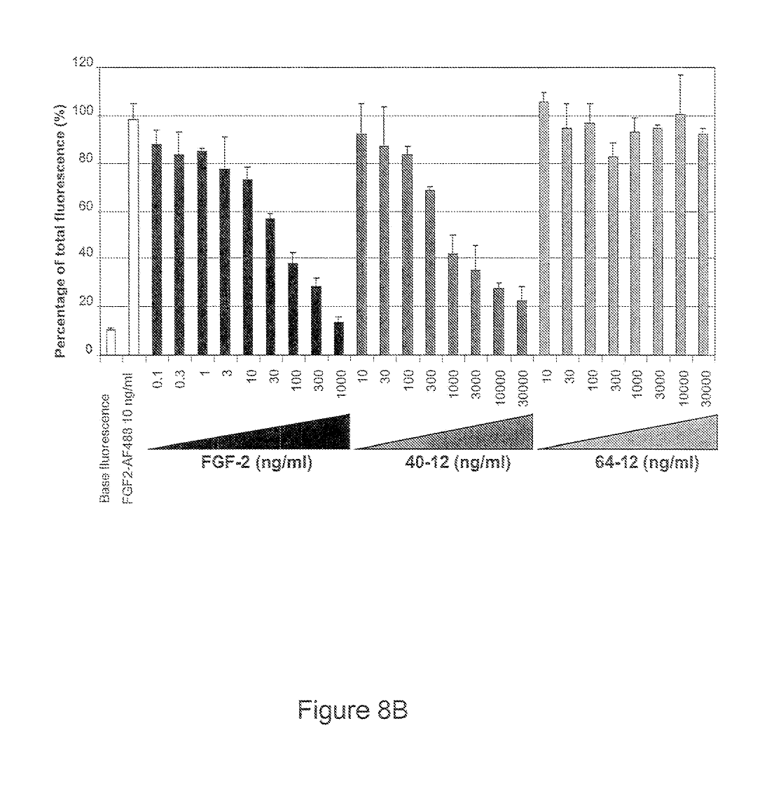

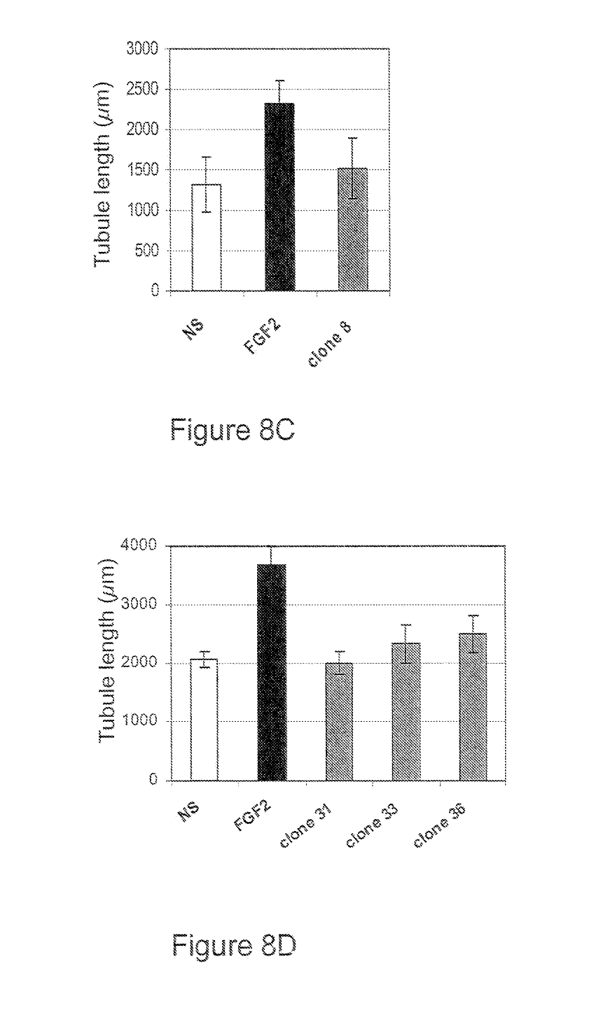

In order to determine the biological activity of the anti-FGFR4 monoclonal antibody 40-12 over the course of human endothelial cell angiogenesis, in vitro angiogenesis experiments were carried out using HUVEC cells stimulated with FGF2 in the presence of the antibody 40-12 or of the control antibody at increasing doses of 1 to 30 μg/ml (FIGS. 8A and 8B).

In this context, the active anti-FGFR4 antagonist monoclonal antibody 40-12 is capable of inhibiting the FGF2-induced angiogenesis of HUVEC cells, at the dose of 30 μg/ml or 200 nM, whereas the control antibody 64-12 has no effect. Furthermore, the antibody 40-12 has no effect per se on the basal angiogenesis.

These results indicate that an FGF-R4-specific antagonist antibody is capable of inhibiting angiogenesis.

As for the antibody 40-12, the anti-FGFR4 antibodies of clones 8, 31, 33 and 36, derived from the phage display, were evaluated with regard to their ability to inhibit the FGF-2-induced angiogenesis of human endothelial cells of HUVEC type. These 4 antibodies block the in vitro stimulation of angiogenesis obtained with FGF-2, said antibodies being at the dose of 10 μg/ml (FIGS. 8C and 8D).

### Example 7

### Antagonistic Effect of the Antibody 40-12 and of the Antibodies Derived from Clones 8, 31, 33 and 36, on Human Hepatocarcinoma Cells (In Vitro)

In order to determine the antitumour effect of the anti-FGFR4 antagonist monoclonal antibody 40-12, experiments were carried out on Hep3b human hepatocarcinoma cells in which the proliferation and the signalling pathways resulting therein are dependent on the ligand-receptor pair: FGF19/FGF-R4.

Firstly, the study of the FGF-R4-dependent signalling pathways resulting in the proliferation of Hep3b cells was undertaken by Western blotting. This cell signalling involves the neosynthesis of the cFos and JunB proteins and also the phosphorylation of Erk1/2 (Lin and al., J Biol. Chem., 2007, 14:27277-84). To do this, 5×10⁵ cells are seeded into dishes 35 mm in diameter, in 2 ml of complete medium (DMEM, 10% FCS, 2 mM glutamine). 24 h later, the cells are subjected to conditions of deficiency for 24 h in 1.8 ml of serum-free medium. The cells are then stimulated for 3 h with 200 μl of 10-times concentrated FGF19, in the absence or in the presence of control anti-FGFR4 antibody or anti-FGFR4 antibody 40-12. The medium is then removed, and the cells are washed once with cold PBS and lysed on the dish for 30 min at 4° C. with 75 μl of RIPA buffer supplemented with protease inhibitor. The total protein extract is then centrifuged for 10 min at 4° C. at 13 000 rpm and the supernatant is analysed by the Western blotting technique. The membranes are then incubated for 2 h at ambient temperature in TBS, 0.05% tween, 5% milk and then the anti-cFos (Cell Signaling Technology, ref 2250), anti-JunB (Cell Signaling Technology, ref 3746) and anti-phospsoErk1/2 (Cell Signaling Technology, ref 4377) primary antibodies are added at 1/1000^{th} and incubated overnight at 4° C. with slow shaking. The membrane is rinsed three times with TBS, 0.05% tween, and the secondary antibody coupled to HRP is incubated for 4 h at 4° C., diluted to 1/2000^{th} in TBS, 0.05% tween, 5% milk. The Western blotting results are then quantified using a Chemigenius machine (Syngene). The intensity of the bands obtained with the various antibodies are weighted with the intensity of the bands obtained with the anti-actin antibody directly coupled to HRP and used at 1/3000^{th} (Santa Cruz Biotechnology, ref Sc-8432-HRP).

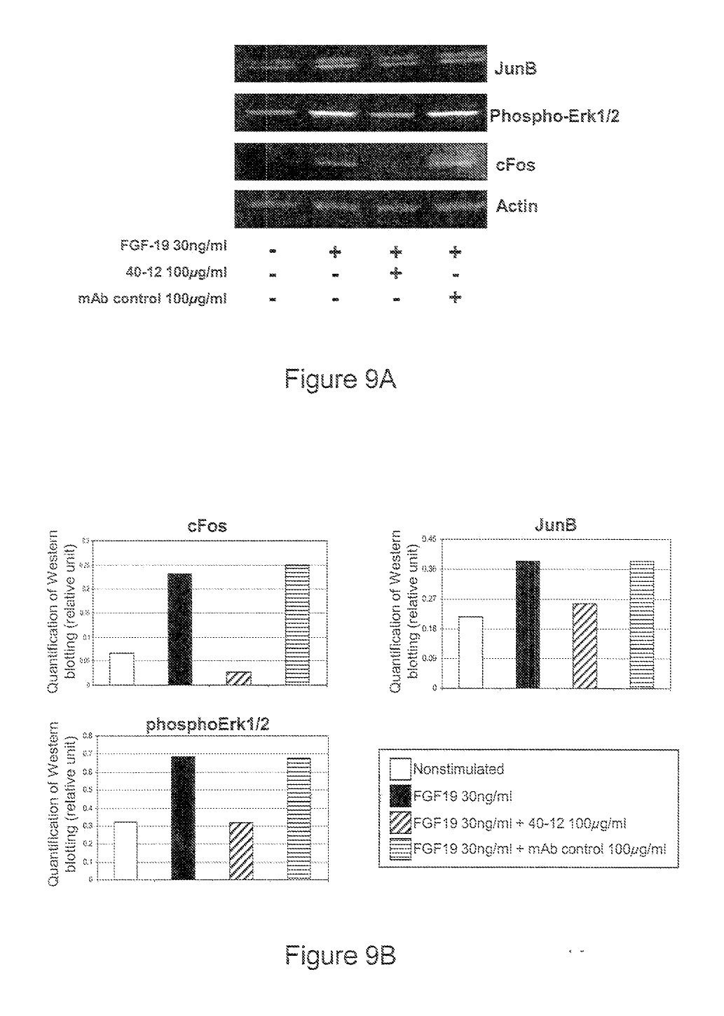

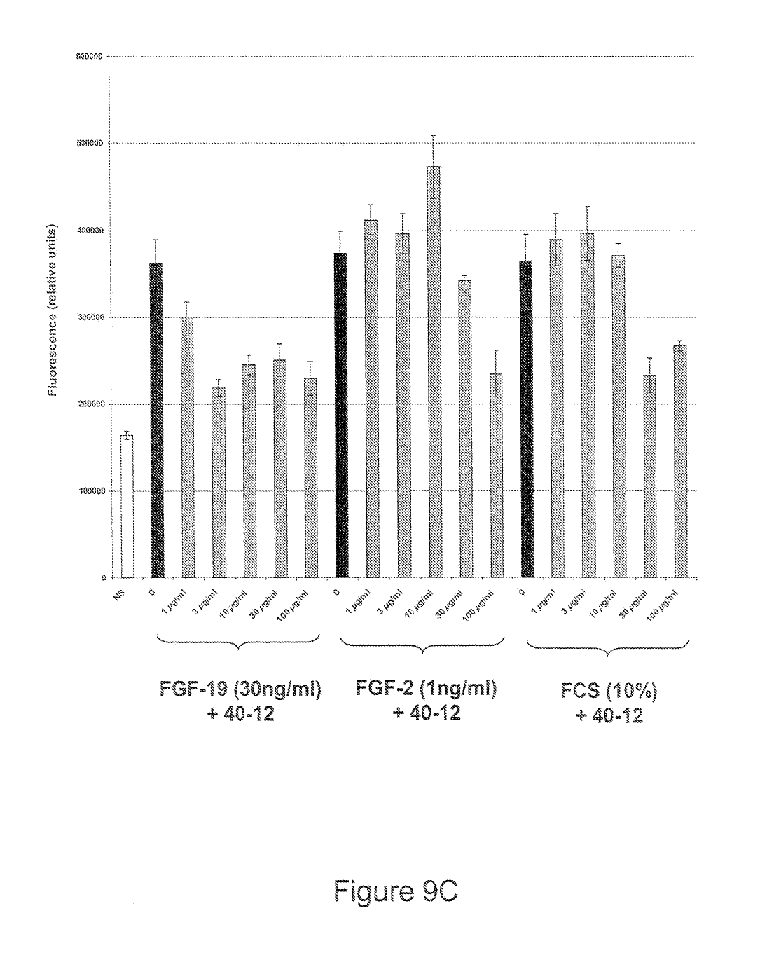

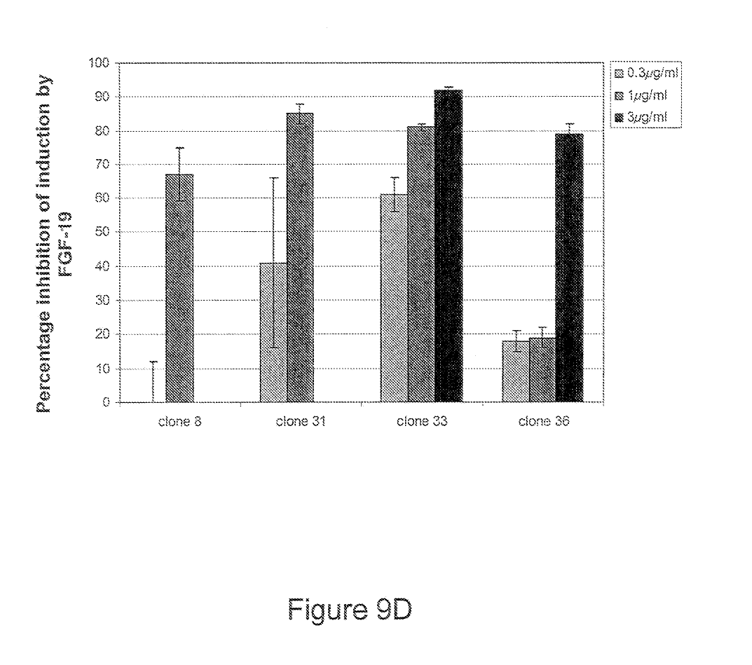

FGF19 at 30 ng/ml induces the synthesis of the JunB and cFos proteins and also the phosphorylation of Erk1/2 in Hep3b cells. This neosynthesis of protein and the phosphorylation of Erk are completely inhibited by the anti-FGFR4 antibody 40-12 at 100 μg/ml, whereas the control antibody has no inhibitory effect. These effects observed on the Western blotting membranes (FIG. 9A) are quantified and the intensities of the bands of each membrane are represented in the form of a graph (FIG. 9B).

Secondly, cell proliferation experiments per se were carried out. 5000 cells are seeded into a 96-well plate in 100 μl of DMEM medium, containing 10% FCS and 2 mM glutamine. 24 h later, the cells are serum deprived in a serum-free culture medium for 24 h. The Hep3b cells are then stimulated for 72 h with 100 μl of serum-free medium supplemented with 10 ng/ml of FGF19 (internal production at Sanofi-Aventis R&D) or with 10% of serum, in the absence or in the presence of control antibodies or of anti-FGFR4 antagonist monoclonal antibody 40-12. After 3 days, the cell proliferation is quantified using the CellTiter Glo kit (Promega, France).

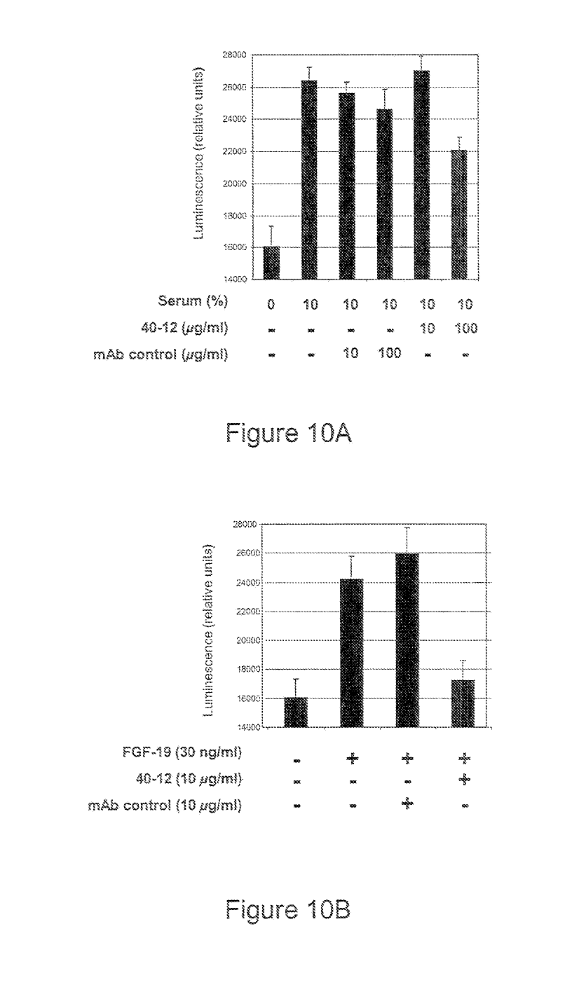

It emerges from these experiments that serum and FGF19 are capable of stimulating Hep3b proliferation. The anti-FGFR4 antagonist antibody 40-12 partially inhibits this serum-induced proliferation at 100 μg/ml, whereas the control antibody does not show any inhibitory activity (FIG. 10A). In addition, the antibody 40-12 at 10 μg/ml completely blocks the proliferation induced by FGF19 (FIG. 10B). The control antibody has no effect.

This demonstrates that the anti-FGFR4 antagonist antibody which is the subject of the invention can be used as an antitumour therapeutic agent in the context of FGF19-dependent or FGF-R4-dependent tumours, and that this antibody would be particularly effective in the treatment of hepatocarcinomas.

In order to simplify the study of the effect of the anti-FGFR4 antibodies on Hep3b cells, an ELISA assay was developed for detecting the phosphorylation of Erk1/2 following the stimulation of these cells with FGF-19 (in correlation with the experiments described in Example 8), FGF-2 or foetal calf serum.

To do this, 50 000 Hep3b cells are seeded into 96-well black clear-bottom plates (COSTAR, ref 3603) in 100 μl of DMEM medium containing 10% FCS and 2 mM glutamine. After 24 h, the cells are subjected to conditions of deficiency for 24 h in FCS-free DMEM medium containing 2 mM glutamine. The medium is then drawn off and replaced with 100 μl of deficiency medium preequilibrated at 37° C., containing FGF or FCS, and also the antibodies evaluated, at the various doses. The cells are incubated for 3 h at 37° C., 5% CO₂. The stimulation medium is then drawn off, the wells are rinsed with PBS at 4° C. and the cells are fixed by adding 200 μl of 4% PFA (paraformaldehyde) in PBS for 15 min at ambient temperature. The PEA is drawn off and the cells are washed three times with 200 μl of PBS. The antibody labelling for detecting phospho-Erk1/2 directly on the Hep3b cells begins by saturating the nonspecific sites with 100 μl of saturation buffer (21.25 ml of PBS, 1.25 ml of 10% non-immune goat serum (Zymed, ref 50-062Z), 75 μl of triton X100) for 2 h. The saturation buffer is replaced with 50 μl of anti-phosphoErk1/2 primary antibody (Cell Signaling Technology, ref 4377) diluted to 1/100^{th} in PBS buffer containing 1% BSA and 0.3% triton X100. The primary antibody is incubated with cells overnight at 4° C. It is then rinsed off with 3 washes of 200 μl of PBS and revealed using an anti-rabbit secondary antibody coupled to AlexaFluor 488 (Molecular Probes, ref A11008) diluted to 1/5000^{th} in PBS buffer containing 1% BSA and 0.3% triton X100 for 4 h. The secondary antibody is then rinsed off with 3 washes of 200 μl of PBS, and 100 μl of PBS is then added to each well. The fluorescence is read over with an EnVision 2103 Multilabel Reader (Perkin Elmer) using the FITC filter.

This technique makes it possible to confirm that FGF-19 at 30 ng/ml induces the phosphorylation of Erk1/2 in Hep3b cells and that 40-12 is capable of blocking this stimulation from doses of 3 μg/ml (FIG. 9C). FGF-2 and the serum are also capable of inducing the system. In the latter 2 cases, the antibody 40-12 inhibits the effect of FGF-2 and of the serum at higher doses (30-100 μg/ml; FIG. 9C).

The detection of phospho-Erk1/2 by ELISA also made it possible to show that anti-FGFR4 antibodies that are active in the in vitro angiogenesis model are also capable of blocking between 70% and 95% of the phosphorylation of Erk1/2 induced by FGF-19 at the dose of 3 μg/ml (FIG. 9D).

Advantageously, the antibodies of the present invention have an antagonistic effect both on the pathological angiogenesis associated with tumour development and on hepatic tumour growth per se, in particular on a model of hepatocarcinoma.

### Example 8

### Antagonistic Effect of the Antibody 40-12 in the Murine Model of Pancreatic Cancer

For this pharmacological model, female Rip1-Tag2 mice (Charles River Laboratory, France) with a C57BI/6J genetic background are used. Starting from week 9 after birth, the animals have drinking water supplemented with 5% sucrose. The mice are treated from week 10 to week 12.5 in an intervention treatment protocol, once a week with a subcutaneous injection of the anti-FGFR4 antibody 40-12 or the control antibody, at the dose of 25 mg/kg (FIG. 14A). This protocol is approved by the “Comité expérimentation Animale (Animal Care and Use Committee)” of Sanofi-Aventis Recherche. Our zootechnical facilities, the attention given to the animals and also the treatment protocols are in accordance with the principles laid down by the European Convention on the protection of vertebrate animals, sacrificed after the period of treatment or when the tumour burden and/or the side effects render their withdrawal from the study obligatory.

For measuring the tumour burden, the animals are sacrificed by euthanasia at the end of the experiment and the tumours are microdissected from freshly excised pancreases. The tumour volume in mm³ is measured using a calliper rule, applying the formula [volume=0.52×(width)²×(length)] in order to approach the volume of a spheroid. The tumour burden per mouse is calculated by the cumulation of the volume of the tumours of each mouse.

For the histochemical analysis of the vascular density, the animals are anaesthetized, and the pancreases are collected, fixed overnight in Accustain® (Sigma) and then embedded in paraffin. Sections 5 μm thick are prepared for each sample. The endothelial cells are detected by incubating the sections with trypsin (Zymed, ref 00-3003) at 37° C. for 10 min, and then with an anti-mouse CD31 antibody, produced in rats, diluted to 1/50^{th} (BD Pharmingen). To reveal the regions labelled with the antibody, the sections are incubated with a biotin-coupled anti-rat antibody for 30 min, and then with HRP-coupled streptavidin, also for 30 min (Vectastain® ABC kit, Vector) and, finally, with DAB for 5 min (Vector, ref SK4100). The sections are then stained with hematoxylin diluted to 1/10^{th} (Dako, S-3309). The photographs are taken with a camera mounted on a microscope (Nikon, E-800) at a total magnification of ×200. The images are analysed using software (Visiolab, Biocom). The blood vessels in the tumour are counted and classified according to their surface area: small vessels between 5 and 20 μm², medium vessels between 21 and 100 μm² and large vessels starting from 101 μm². Two slides per pancreas are analysed in order to determine the vascular density corresponding to the total number of elements labelled per field.

In this model, the subcutaneous treatment using the anti-FGFR4 antibody 40-12 at 25 mg/kg once a week between the tenth and twelfth weeks makes it possible to significantly reduce the tumour burden by 55% (FIG. 11B) and has a tendency to reduce the number of tumours per pancreas by 34% (FIG. 11C), whereas the control treatment has no effect. This inhibition of tumour development by virtue of the anti-FGFR4 antibody 40-12 is accompanied by a significant reduction of 31% in the total vascular density (FIG. 11D) corresponding to an observed reduction in the number of blood vessels in all the vessel-size groups, labelled with an anti-CD31 antibody (FIG. 11D).

These in vivo results clearly show that an anti-FGFR4 antagonist antibody is capable of inhibiting the recruitment and the formation of blood vessels in the tumour. This inhibition of tumour vascularization is accompanied by a reduction in the number of tumours per pancreas and in the total tumour volume.

Advantageously, the antibodies of the present invention have an antagonistic effect both on pathological angiogenesis and on hepatic tumour growth (on a model of hepatocarcinoma) and pancreatic tumour growth.

### Example 9

### Cross-Reactivity of the Antibody with Human, Murine and Rat FGF-R4s

Firstly, competition binding experiments were carried out. To do this, FGF2 was labelled on the 2 free cysteines with an AlexaFluor® 488 nm C-5 maleimide (Molecular Probes, A10254) according to the supplier's recommendations. This FGF2-AF488, at 10 ng/ml, is capable of binding to the human FGF-R4s expressed at the surface of transfected 300-19 cells (FIG. 8). This binding is specific since the addition of excess unlabelled FGF2 makes it possible to displace the FGF2/FGF-R4 interaction (FIG. 8A). The same experiment was carried out with increasing doses of the antibody 40-12 or of the control antibody 64-12 being added. Only the antagonist antibody 40-12 is capable of displacing the FGF2-AF488/FGF-R4 binding, with an IC₅₀ of 3500 ng/ml, i.e. 23 nM, showing that the antagonistic effect of this antibody is due to its ability to displace FGF/FGF-R4 binding.

Secondly, and for the purpose of determining the ability of clone 40-12 to block FGF/FGF-R4 binding in species other than humans, a dissociation experiment was carried out using the abovementioned mouse, rat and human FGF2/FGF-R4 pairs. To do this, murine FGF2 (R&D, ref 3139-FB-025) or rat FGF2 (R&D, ref 3339-FB-025) was labelled in the same way as human FGF2, with AlexaFluor 488. The dissociation experiments were carried out using the 300-19 lines transfected with mouse or rat FGF-R4 receptors. The results show that the anti-FGFR4 antagonist antibody 40-12 is capable of dissociating the FGF2/FGF-R4 binding in a mouse or rat system, with the same effectiveness as in the human system. In fact, the IC₅₀ values are 3500, 4110 and 3940 ng/ml, i.e. 23, 27 and 26 nM, for the human, murine or rat FGF2/FGF-R4 complexes, respectively (FIGS. 13A, 13B and 13C, respectively). This ability to bind to rodent FGF-R4 was verified by ELISA. The antibody 40-12 binds both to human FGF-R4 and to murine FGF-R4 (FIG. 12).

These results indicate that the anti-FGFR4 antibody 40-12 may be used in pharmacological models on rodents (at least mice and rats) and that the results obtained should be predictive of the effectiveness in humans.

Similarly, studies carried out on the antibodies derived from clones 8, 31, 33 and 36 show that these antibodies recognize both human FGF-R4 and murine FGF-R4 (Table 4 below)

TABLE 4
Kinetic parameters for binding of the anti-FGFR4 antibodies 8, 31,
33 and 36, measured by Surface Plasmon Resonance (BIAcore 3000):

| | on h-FGFR4-Histag | | | on m-FGFR4-Histag | | |
|---|---|---|---|---|---|---|
| | Kon (M⁻¹s⁻¹) | Koff (s⁻¹) | KD (M) | Kon (M⁻¹s⁻¹) | Koff (s⁻¹) | KD (M) |
| Clone 8 | 8.93 × 10⁵ | 2.92 × 10⁻⁴ | 3.27 × 10⁻¹⁰ | 1.20 × 10⁶ | 1.36 × 10⁻⁴ | 1.15 × 10⁻¹⁰ |
| Clone 31 | 6.48 × 10⁵ | 9.80 × 10⁻⁴ | 1.52 × 10⁻⁹ | 9.14 × 10⁵ | 1.80 × 10⁻³ | 1.97 × 10⁻⁹ |
| Clone 33 | 8.05 × 10⁵ | 6.17 × 10⁻⁴ | 7.71 × 10⁻¹⁰ | 1.01 × 10⁶ | 7.04 × 10⁻⁴ | 6.92 × 10⁻¹⁰ |
| Clone 36 | 2.44 × 10⁵ | 6.31 × 10⁻⁴ | 2.62 × 10⁻⁹ | 3.30 × 10⁵ | 7.52 × 10⁻⁴ | 2.28 × 10⁻⁹ |

### Example 10

### Determination of the Epitope Recognized by the Anti-FGF-R4 Antibodies

### A—Determination of the Epitope Recognized by the Anti-FGFR4 Antibody 40-12

A screen was carried out in order to determine the specific domain of FGFR4 recognized by the antibody 40-12, by ELISA assay. Through the use of a deleted form of the D1 domain of FGF-R4, in ELISA, it was established that the antibody 40-12 interacted with the D2-D3 domains of FGF-R4 (FIG. 12).

A second screen was carried out by ELISA assay, using, as capture antigen, the constructs containing either the D1 domain (SABVA4794) or the D2 domain (SABVA4796) or the D3 domain (SABVA4799) of the hFGFR4 protein, as described in Example 2. The capture antigen was bound to Immulon-4 enzyme-linked plates (VWR Scientific Inc. Swedesboro, N.J.). The hybridoma 40-12 was subsequently added and then detection was carried out using the peroxidase-conjugated anti-mouse IgG rabbit antibody (Sigma; ref. A9044-dilution to 1:50 000). The revealing was carried out with the TMB-H2O2 substrate (Interchim; ref UP664780) and the optical density (OD) measurements were carried out at 450 nm. Table 5 summarizes the results obtained:

TABLE 5
Measurement of the binding of the antibody
40-12 to the various subdomains of FGF-R4

| | D1 | D2 | D3 | D1-D3 |
|---|---|---|---|---|
| Domain studied | SABVA4794 | SABVA4796 | SABVA4799 | FGFR4-Fc |
| Signal | 0.185 | 3.000 | 0.105 | 3.000 |
| (O.D., 450 nm) | | | | |

The anti-FGFR4 antibody therefore recognizes the D2 domain of the extracellular portion of the FGFR4 protein.

Furthermore, the FGFR4-Fc protein denatured with FCS is not recognized by the antibody 40-12 in Western blotting analysis, thereby indicating that the epitope targeted by 40-12 on the D2 domain of FGFR4 is of conformational type.

### B—Determination of the Epitope Recognized by the Anti-FGFR4 Antibodies 8, 31, 33, 36.

A screen was carried out in order to determine the specific domain of FGFR4 recognized by the antibodies 8, 31, 33 and 36, by ELISA assay using, as capture antigen, the constructs containing either the D2 and D3 domains (SEQ ID No. 42) or the hFGFR4-Histag protein (SABVA4614, SEQ ID No. 40). The capture antigen was bound to Immulon-4 enzyme-linked plates (VWR Scientific Inc. Swedesboro, N.J.). Culture supernatants from HEK293 cells transiently transfected with plasmids which enable the secretion of the antibodies 8, 31, 33 and 36 were subsequently added and then detection was carried out using the peroxidase-conjugated anti-human IgG rabbit antibody (DakoCytomation, ref. P0214-dilution to 1:5000). The revealing was carried out with the TMB-H2O2 substrate (Interchim; ref UP664780) and the optical density (OD) measurements were carried out at 450 nm. Table 6 below summarizes the results obtained:

TABLE 6
Binding of the antibodies derived from clones 8, 31, 33 and 36
to whole FGF-R4 or to the D2-D3 subdomain (OD signal at 450 nm)

| | hFGFR4-Histag | hFGFR4(D2,D3)-Histag |
|---|---|---|
| Clone 8 | 3.898 | 3.860 |
| Clone 31 | 3.859 | 3.741 |
| Clone 33 | 3.752 | 3.621 |
| Clone 36 | 3.751 | 3.616 |

The antibodies 8, 31, 33 and 36 therefore recognize the D2-D3 domain of the extracellular portion of the FGFR4 protein.

TABLE 7
Sequences used, obtained and deduced

| | |
|---|---|
| Nucleotide | Protein |
| sequences | sequences |

Antibody 40-12

| | | |
|---|---|---|
| VH + CH | SEQ ID No. 1 | SEQ ID No. 2 |
| VL + CL | SEQ ID No. 3 | SEQ ID No. 4 |
| VH | SEQ ID No. 5 | SEQ ID No. 6 |
| VL | SEQ ID No. 7 | SEQ ID No. 8 |
| CDR VH | | SEQ ID No. 9; 10; 11 |
| CDR VL | | SEQ ID No. 12; 13; 14 |

Antibody 64-12

| | | |
|---|---|---|
| VH + CH | SEQ ID No. 15 | SEQ ID No. 16 |
| VL + CL | SEQ ID No. 17 | SEQ ID No. 18 |
| VH | SEQ ID No. 19 | SEQ ID No. 20 |
| VL | SEQ ID No. 21 | SEQ ID No. 22 |
| CDR VH | | SEQ ID No. 23; 24; 25 |
| CDR VL | | SEQ ID No. 26; 27; 28 |

Humanized sequences

| | | |
|---|---|---|
| Light chain 1 | SEQ ID No. 29 | SEQ ID No. 30 |
| Light chain 2 | SEQ ID No. 31 | SEQ ID No. 32 |
| Heavy chain 1 | SEQ ID No. 33 | SEQ ID No. 34 |
| Heavy chain 2 | SEQ ID No. 35 | SEQ ID No. 36 |
| Heavy chain 3 | SEQ ID No. 37 | SEQ ID No. 38 |

Constructs

| | | |
|---|---|---|
| hFGFR4-Histag | SEQ ID No. 39 | SEQ ID No. 40 |
| hFGFR4-Streptag | SEQ ID No. 68 | SEQ ID No. 69 |
| hFGFR4(D2D3)- | SEQ ID No. 41 | SEQ ID No. 42 |
| Histag | | |
| mFGFR4-Histag | SEQ ID No. 43 | SEQ ID No. 44 |
| hFGFR1-Fc | SEQ ID No. 45 | SEQ ID No. 46 |
| hFGFR2-Fc | SEQ ID No. 47 | SEQ ID No. 48 |
| rFGFR4 | SEQ ID No. 49 | SEQ ID No. 50 |
| Primer sequences for | SEQ ID No. 51; 52; | |
| establishing clonal line | 53; 54; 55; 56; 58 | |
| (Example 4) | SEQ ID No. 61; 62 | |
| FGFR4 | | SEQ ID No. 59; 60 |
| transmembrane | | |
| domain | | |
| Primer | SEQ ID No. 63; 64; | |
| oligonucleotides | 65; 66; 67 | |
| Table 1 | | |
| Epitope in D2 domain | | SEQ ID No. 70 |

Antibody clone 8

| | | |
|---|---|---|
| Whole light chain | SEQ ID No. 71 | SEQ ID No. 72 |
| Light chain CDR | | SEQ ID No. 73; 74; 75 |
| Whole heavy chain | SEQ ID No. 76 | SEQ ID No. 77 |
| Heavy chain CDR | | SEQ ID No. 78; 79; 80 |

Antibody clone 31

| | | |
|---|---|---|
| Whole light chain | SEQ ID No. 81 | SEQ ID No. 82 |
| Light chain CDR | | SEQ ID No. 83; 84; 85 |
| Whole heavy chain | SEQ ID No. 86 | SEQ ID No. 87 |
| Heavy chain CDR | | SEQ ID No. 88; 89; 90 |

Antibody clone 33

| | | |
|---|---|---|
| Whole light chain | SEQ ID No. 91 | SEQ ID No. 92 |
| Light chain CDR | | SEQ ID No. 93; 94; 95 |
| Whole heavy chain | SEQ ID No. 96 | SEQ ID No. 97 |
| Heavy chain CDR | | SEQ ID No. 98; 99; 100 |

Antibody clone 36

| | | |
|---|---|---|
| Whole light chain | SEQ ID No. 101 | SEQ ID No. 102 |
| Light chain CDR | | SEQ ID No. 103; 104; 105 |
| Whole heavy chain | SEQ ID No. 106 | SEQ ID No. 107 |
| Heavy chain CDR | | SEQ ID No. 108; 109; 110 |

Subdomains of the extracellular portion of hFGFR4, fused to
the Fc domain of IgG1

| | | |
|---|---|---|
| fGFGR4_D1::Fc | SEQ ID No. 111 | SEQ ID No. 112 |
| fGFGR4_D2::Fc | SEQ ID No. 113 | SEQ ID No. 114 |
| fGFGR4_D3::Fc | SEQ ID No. 115 | SEQ ID No. 116 |
| Human IgG1 constant | | SEQ ID No. 117 |
| region sequence | | |

1. FGF-R4 receptor antagonist, characterized in that it is an antibody that specifically binds to FGF-R4.

2. Antagonist according to claim 1, characterized in that it binds to the D2-D3 domain of the FGF-R4 receptor.

3. Antibody according to either of claims 1 and 2, characterized in that it binds to the D2 domain of the FGF-R4 receptor.

4. Antagonist according to one of the preceding claims, characterized in that it has a K_{D} with respect to the FGF-R4 receptor, determined by the Surface Plasmon Resonance (Biacore) technique, of less than 10⁻⁸ M.

5. Antagonist according to one of the preceding claims, characterized in that it is active both against human FGF-R4 and against murine FGF-R4.

6. Antagonist according to one of the preceding claims, characterized in that it is an antibody and comprises at least one CDR having a sequence identical to one of the sequences SEQ ID No. 9, 10, 11, 12, 13, 14, 73, 74, 75, 78, 79, 80, 83, 84, 85, 88, 89, 90, 93, 94, 95, 98, 99, 100, 103, 104, 105, 108, 109 or 110.

7. Antagonist according to one of the preceding claims, characterized in that it is an antibody and comprises the CDRs of sequence SEQ ID Nos. 9, 10, 11, 12, 13 and 14; or 73, 74, 75, 78, 79 and 80; or 83, 84, 85, 88, 89 and 90; or 93, 94, 95, 98, 99 and 100; or 103, 104, 105, 108, 109 and 110, where one of the CDRs may differ by one or two amino acids compared with at least one of the sequences mentioned above, provided that the antibody keeps its binding specificity.

8. Antagonist according to one of the preceding claims, characterized in that it is an antibody of which the variable region of its heavy chain comprises a nucleotide sequence having at least 80% identity with the sequence SEQ ID No. 5, 76, 86, 96 or 106.

9. Antagonist according to one of the preceding claims, characterized in that it is an antibody of which the variable region of its light chain comprises a nucleotide sequence having at least 80% identity with the sequence SEQ ID No. 7, 71, 81, 91 or 101.

10. Antagonist according to one of the preceding claims, characterized in that it is an antibody and in that its sequence comprises the polypeptide sequences SEQ ID Nos. 2 and 4; or 6 and 8; or 72 and 77; or 82 and 87; or 92 and 97; or 102 and 107.

11. Antagonist according to any one of the preceding claims, characterized in that it induces inhibition of the cell signalling pathways controlled by FGF-R4.

12. Antagonist according to any one of the preceding claims, characterized in that it induces inhibition of angiogenesis.

13. Antagonist according to any one of the preceding claims, characterized in that it induces inhibition of tumour cell proliferation.

14. Antagonist according to any one of the preceding claims, characterized in that its affinity for FGF-R4 is 10 times greater than its affinity for the other FGF receptors.

15. Antagonist according to any one of claims 1 to 7, characterized in that it is a humanized antibody.

16. Antagonist according to any one of claims 1 to 7, characterized in that it is a human antibody.

17. Antagonist according to claim 15, characterized in that it comprises a variable light chain having at least 80% identity with one of the polypeptide sequences SEQ ID No. 30 or 32.

18. Antagonist according to claim 15, characterized in that it comprises a variable heavy chain having at least 80% identity with a sequence SEQ ID No. 34, 36 or 38.

19. Antagonist according to any one of the preceding claims, characterized in that it is an antibody and in that it is conjugated to a cytotoxic agent.

20. Use of an antagonist according to any one of the preceding claims, in the treatment of diseases associated with a pathological angiogenesis.

21. Use of an antagonist according to any one of the preceding claims, in the treatment of hepatocarcinomas or of any other type of hepatic cancer.

22. Use of an antagonist according to any one of the preceding claims, in the treatment of pancreatic cancer.

23. Pharmaceutical composition comprising an antagonist according to any one of claims 1 to 19 and excipients.

24. Method of treating a disease related to a pathological angiogenesis, characterized in that it comprises the administration, to the patient, of an antibody according to any one of claims 1 to 19.

25. Method of treating a cancer, characterized in that it comprises the administration, to the patient, of an antibody according to any one of claims 1 to 19.

26. Cell line producing antibodies according to claims 1 to 19.

27. Method of producing an antibody according to any one of claims 1 to 19, characterized in that it comprises culturing a cell line according to claim 26.

28. Drug comprising an antagonist according to any one of claims 1 to 19.

29. Polynucleotide encoding a polypeptide having at least 80% identity with one of the sequences SEQ ID No. 2, 4, 6, 8, 9, 10, 11, 12, 13, 14, 30, 32, 34, 36, 38, 72, 73, 74, 75, 77, 78, 79, 80, 82, 83, 84, 85, 87, 88, 89, 90, 92, 93, 94, 95, 97, 98, 99, 100, 103, 104, 105, 107, 108, 109 or 110.

30. Polynucleotide characterized in that it has a sequence having at least 80% identity with one of the sequences SEQ ID No. 1, 3, 5, 7, 29, 31, 33, 35, or 37, 71, 76, 81, 86, 91, 96, 101 or 106.

31. Recombinant vector comprising a nucleic acid according to either one of claims 29 and 30.

32. Host cell comprising a vector according to claim 31.
